(19) United States
(12) Patent Application Publication
(10) Pub. No.: US 2013/0096149 A1
(43) Pub. Date: Apr. 18, 2013

(54) HETEROARYL COMPOUNDS AND COMPOSITIONS AS PROTEIN KINASE INHIBITORS

(71) Applicants: Ann Marie Madera, Dublin, CA (US); Daniel Poon, Piedmont, CA (US); Aaron Smith, Fremont, CA (US)

(72) Inventors: Ann Marie Madera, Dublin, CA (US); Daniel Poon, Piedmont, CA (US); Aaron Smith, Fremont, CA (US)

(73) Assignee: NOVARTIS AG, Basel (CH)

(21) Appl. No.: 13/805,793

(22) PCT Filed: Jun. 23, 2011
(86) PCT No.: PCT/EP11/60561

§ 371 Date: Dec. 20, 2012

Related U.S. Application Data
(60) Provisional application No. 61/358,603, filed on Jun. 25, 2010.
(60) Provisional application No. 61/368,534, filed on Jul. 28, 2010.

(51) Int. Cl. A61K 31/506; A61K 45/06; A61K 31/4439; C07D 417/04; C07D 413/04

(52) U.S. Cl. CPC A61K 31/506; C07D 417/04; C07D 413/04; A61K 31/4439; A61K 45/06
USPC 514/275; 544/331; 544/333; 514/256; 546/270.4; 514/342; 546/271.4; 514/340

(57) ABSTRACT

The present invention provides compounds of Formula I:

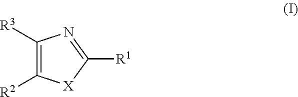

wherein R¹, R², R³, and X are as defined herein. The compounds of Formula (I) and pharmaceutical compositions thereof are useful for the treatment of cancer, and B-Raf-associated diseases.

### FIELD OF THE INVENTION

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of B-Raf.

### BACKGROUND

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGF-R), the nerve growth factor receptor, trkB, Met, and the fibroblast growth factor receptor, FGFR3; non-receptor tyrosine kinases such Abl and the fusion kinase BCR-Abl, Lck, Csk, Fes, Bmx and c-src; and serine/threonine kinases such as B-Raf, sgk, MAP kinases (e.g., MKK4, MKK6, etc.) and SAPK2α, SAPK2β and SAPK3. Aberrant kinase activity has been observed in many disease states including benign and malignant proliferative disorders as well as diseases resulting from inappropriate activation of the immune and nervous systems.

### SUMMARY

The present invention provides a compound of Formula I:

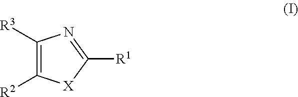

or a pharmaceutically acceptable salt thereof, wherein:

X represents O or S;

R¹ is selected from C₁₋₆-alkyl, C₃₋₈ branched alkyl, C₃₋₈ cycloalkyl, optionally substituted heteroaryl, optionally substituted heterocyclyl, and optionally substituted aryl;

R² is heteroaryl substituted with R¹¹;

R³ is selected from phenyl substituted with R¹², R¹³, and R¹⁵;

R¹¹ is selected from H, and optionally substituted amino;

R¹² is halogen or H;

R¹³ is selected from NHSO₂alkyl, and NHSO₂aryl; and

R¹⁵ is selected from halogen, H, and C₁₋₆ alkyl.

A preferred embodiment of the present invention provides a compound of Formula I, or a pharmaceutically acceptable salt thereof, wherein X represents O or S;

R¹ is selected from C₃₋₆ branched alkyl, C₃₋₆ cycloalkyl, and optionally substituted phenyl; R² is heteroaryl substituted with R¹¹; R³ is selected from phenyl substituted with R¹², R¹³, and R¹⁵; R¹¹ is selected from H, amino, and NH—CH₂—CH(CH₃)NH—C(O)—OCH₃; R¹² is halogen; R¹³ is selected from NHSO₂—C₁₋₆ alkyl, and NHSO₂-optionally substituted phenyl; and R¹⁵ is selected from halogen, H, and C₁₋₆ alkyl.

Yet another preferred embodiment provides a compound of Formula I, or a pharmaceutically acceptable salt thereof, wherein X represents O or S; R¹ is selected from C₃₋₆ branched alkyl, C₃₋₆ cycloalkyl, and optionally substituted phenyl; R² is heteroaryl substituted with R¹¹; R³ is selected from phenyl substituted with R¹², R¹³, and R¹⁵; R¹¹ is selected from H, NH—(CH₂)₁₋₂—CN, and amino;

R¹² is halogen; R¹³ is NHSO₂—C₁₋₆ alkyl; and R¹⁵ is selected from halogen, H, and C₁₋₆ alkyl. A preferred aspect of this embodiment provides a compound of Formula I wherein X represents O or S; R¹ is selected from t-butyl, cyclo-propyl, and substituted phenyl; R² is pyrimidinyl substituted with R¹¹; R³ is selected from phenyl substituted with R¹², R¹³, and R¹⁵; R¹¹ is NH₂; R¹² is Cl or F; R¹³ is NHSO₂—C₁₋₃ alkyl; and R¹⁵ is selected from F, Br, CH₃, H, and Cl. A particularly aspect of this aspect of the presentation provides a compound of Formula I wherein X represents O; R¹ represents cyclopropyl; and

R¹⁵ represents Cl or F. Another particularly preferred aspect of the present invention provides a compound of Formula I wherein X represents S, R¹ represents cyclopropyl; and R¹⁵ represents Cl or F.

A further preferred embodiment of the present invention provides a compound of Formula I, or a pharmaceutically acceptable salt thereof, wherein:

X represents O or S; R¹ is selected from C₃₋₆ branched alkyl, C₃₋₆ cycloalkyl, and optionally substituted phenyl; R² is heteroaryl substituted with R¹¹;

R³ is selected from phenyl substituted with R¹², R¹³, and R¹⁵; R¹¹ is NH(CH₂)₁₋₂—CN, or NH—CH₂—CH(CH₃)NH—C(O)—OCH₃; R¹² is halogen; R¹³ is NHSO₂-substituted phenyl; and R¹⁵ is selected from halogen, H, and C₁₋₆ alkyl. A further preferred aspect of this embodiment provides a compound of Formula I wherein X represents O or S; R¹ is selected from t-butyl, cyclo-propyl, and substituted phenyl; R² is pyrimidinyl substituted with R¹¹; R³ is selected from phenyl substituted with R¹², R¹³, and R¹⁵; R¹¹ is NH(CH₂)₁₋₂—CN, or NH—CH₂—CH(CH₃)NH—C(O)—OCH₃; R¹² is Cl or F; R¹³ is NHSO₂-substituted phenyl; and R¹⁵ is selected from F, Br, CH₃, H, and Cl.

A particularly preferred compounds of the present invention is selected from the group consisting of:
N-(3-(5-(2-aminopyrimidin-4-yl)-2-cyclopropylthiazol-4-yl)-5-chloro-2-fluorophenyl)propane-1-sulfonamide;
(S)-methyl 1-(4-(4-(5-chloro-2-fluoro-3-(propylsulfonamido)phenyl)-2-cyclopropylthiazol-5-yl)pyrimidin-2-ylamino)propan-2-ylcarbamate;
N-(2,5-dichloro-3-(2-cyclopropyl-5-(2-(methylamino)pyrimidin-4-yl)thiazol-4-yl)phenyl)propane-1-sulfonamide;
N-(3-(5-(2-aminopyrimidin-4-yl)-2-cyclopropylthiazol-4-yl)-2,5-dichlorophenyl)propane-1-sulfonamide;
(S)-methyl 1-(4-(4-(2-chloro-5-fluoro-3-(propylsulfonamido)phenyl)-2-cyclopropylthiazol-5-yl)pyrimidin-2-ylamino)propan-2-ylcarbamate; and
N-(2-chloro-3-(2-cyclopropyl-5-(2-(methylamino)pyrimidin-4-yl)thiazol-4-yl)-5-fluorophenyl)propane-1-sulfonamide;
N-(3-(5-(2-aminopyrimidin-4-yl)-2-cyclopropyloxazol-4-yl)-5-chloro-2-fluorophenyl)propane-1-sulfonamide; and
N-(3-(5-(2-aminopyrimidin-4-yl)-2-cyclopropyloxazol-4-yl)-2,5-dichlorophenyl)propane-1-sulfonamide

or a pharmaceutically acceptable salt thereof.

In another aspect of the present invention, a pharmaceutical composition is provided which comprises a compound of Formula (I), or a pharmaceutically acceptable salt thereof, and a diluent, carrier or excipient. The pharmaceutical composition may further comprise an additional therapeutic agent, wherein the additional therapeutic agent is selected from the group consisting of an anticancer compound, an analgesic, an antiemetic, an antidepressant, and an anti-inflammatory agent.

In yet another aspect of the present invention provides a method for treating cancer comprising administering to a subject in need of such treatment a pharmaceutically effective amount of a compound of Formula (I), or a pharmaceutical acceptable salt thereof. A preferred embodiment of this aspect provides a method wherein said cancer is selected from the group consisting of lung carcinoma, pancreatic carcinoma, bladder carcinoma, colon carcinoma, myeloid disorders, prostate cancer, thyroid cancer, melanoma, and adenomas.

Another aspect of the present invention provides a method for treating cancer comprising administering to a subject in need of such treatment a pharmaceutically effective amount of a compound of Formula (I), or a pharmaceutical acceptable salt thereof, and a diluent, carrier or excipient. A preferred embodiment of this aspect provides a method wherein said cancer is selected from the group consisting of lung carcinoma, pancreatic carcinoma, bladder carcinoma, colon carcinoma, myeloid disorders, prostate cancer, thyroid cancer, melanoma, and adenomas.

In another aspect of the present invention is provided a method for treating a condition mediated by Raf kinase which comprises administering to a subject in need thereof an effective amount of a compound of Formula (I), or a pharmaceutical acceptable salt thereof, or a pharmaceutical composition comprising a compound of Formula (I), or a pharmaceutical acceptable salt thereof, and a diluent, carrier or excipient. Preferably, the Raf Kinase being mediated is a mutant b-Raf kinase, more preferably, a mutant b-Raf(V600E) kinase.

The methods may comprise administering an additional therapeutic agent. Preferred additional agents include an anticancer drug, a pain medication, an antiemetic, an antidepressant or an anti-inflammatory agent, more preferably, the additional therapeutic agent is a different Raf kinase inhibitor or an inhibitor of MEK, mTOR, PI3K, CDK9, PAK, Protein Kinase C, a MAP kinase, a MAPK Kinase, or ERK.

The invention can be further illustrated with the following non-limiting examples which are provided for illustrative purposes only and are not to be construed as limiting upon teachings herein, in which:

### DEFINITIONS

“Alkyl” as a group and as a structural element of other groups, for example halo-substituted-alkyl and alkoxy, can be either straight-chained or branched. C₁₋₄-alkoxy includes, methoxy, ethoxy, and the like. “Halosubstituted alkyl” refers to an alkyl group (branched or unbranched) wherein any of the hydrogens can be substituted with a halogen. Representative examples of halosubstituted-(C₁-C₄)alkyl include fluoromethyl, difluoromethyl, trifluoromethyl, chlorofluoromethyl, difluoroethyl, pentafluoroethyl, and the like. Similarly, hydroxy-substituted-(C₁-C₆)alkyl means and alkyl group (branched or unbranched) wherein any of the hydrogens can be substituted with a hydroxyl. For example, hydroxy-substituted-(C₁-C₆)alkyl includes 2-hydroxyethyl, and the like. Similarly, cyano-substituted-(C₁-C₆)alkyl means and alkyl group (branched or unbranched) wherein any of the hydrogens can be substituted with cyano.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl above where one or more of the ring members is a heteroatom. For example, (C₁-C₁₀)heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, (C₃-C₁₀)cycloalkyl includes cyclopropyl, cyclobutyl, cyclopentyl, cyclohexyl, cycloheptyl, cyclooctyl, cyclononyl, and cyclodecyl. A preferred cycloalkyl is cyclopropyl.

“Heterocycloalkyl” means cycloalkyl where one or more of the ring carbons is replaced by a moiety selected from —O—, —N═, —NR—, —C(O)—, —S—, —S(O)— or —S(O)₂—, wherein R is hydrogen, (C₁-C₄)alkyl or a nitrogen protecting group (—NPg). Representative examples of (C₃-C₈)heterocycloalkyl include 2H-pyranyl, 4H-pyranyl, piperidinyl, 1,4-dioxane, morpholinyl, 1,4-dithianyl, thiomorpholino, imidazolidin-2-one, tetrahydrofuran, piperazinyl, 1,3,5-trithianyl, pyrrolidinyl, pyrrolidinyl-2-one, piperidinone, 1,4-dioxa-8-aza-spiro[4.5]dec-8-yl, etc.

“Halogen” (or halo) represents chloro, fluoro, bromo or iodo.

“pMEK” means phosphorylated Mek.

“pERK” means phosphorylated ERK.

“Treat”, “treating” and “treatment” refer to a method of alleviating or abating a disease and/or its attendant symptoms.

The term “compounds of the present invention” (unless specifically identified otherwise) refer to compounds of Formula (I), prodrugs thereof, pharmaceutically acceptable salts of the compounds, and/or prodrugs, and hydrates or solvates of the compounds, salts, and/or prodrugs, as well as, all stereoisomers (including diastereoisomers and enantiomers), tautomers and isotopically labeled compounds.

### DETAILED DESCRIPTION

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der Organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### GENERAL SYNTHETIC DESCRIPTION

Compounds of Formula (I) can be prepared using the procedure outlined in Scheme I below.

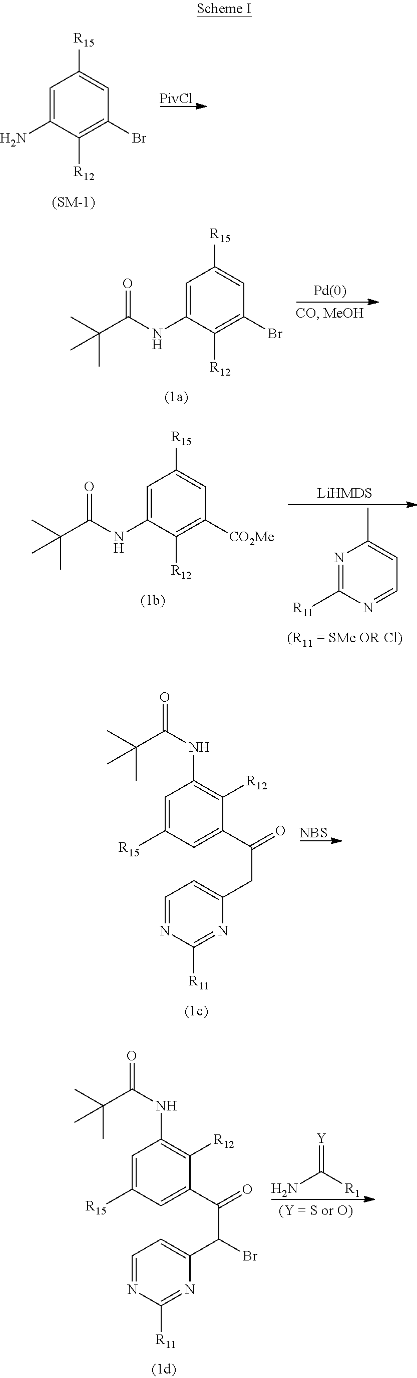

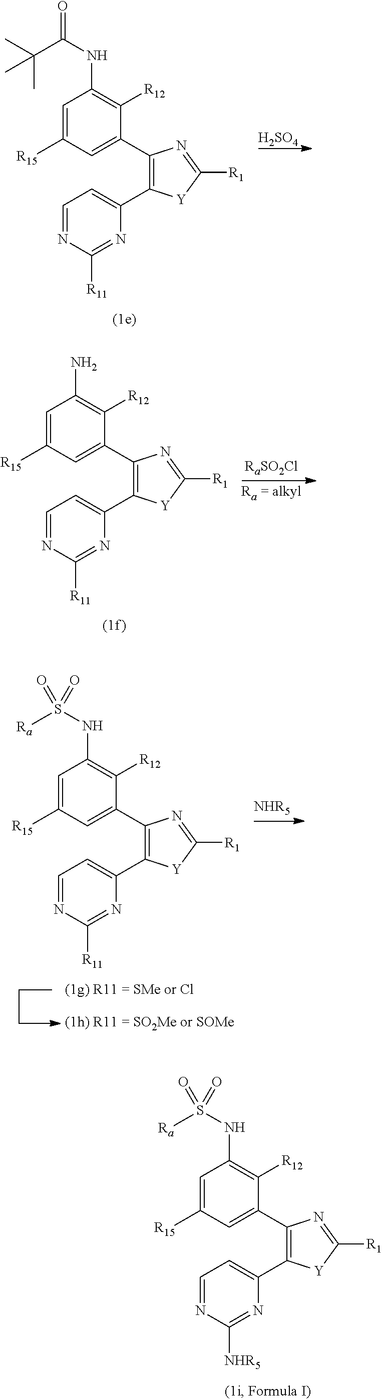

Simple protection of starting bromoaniline (SM-1) as the pivalamide (1a) followed by Pd(0) mediated carbonylation of the bromide moiety can provide the functionalized amidoester (1b). Deprotonation of 4-methyl-2-(methylthio)pyrimidine or 4-methyl-2-chloropyrimidine followed by addition to (1b) could furnished the elaborated ketone (1c) whereupon treatment with N-bromosuccinimide (NBS) would yield the bromoketone (1d). Cyclocondensation with an appropriately substituted thioamide or amide would then result in the corresponding thiazole or oxazole (1e). Removal of the pivalamide protecting group and treatment of the subsequent aniline (1f) with the desired sulfonyl chloride would produce intermediate (1g). The direct displacement of the 2-chloropyrimidine moiety or oxidation of the methylthio moiety to the sulfone or sulfoxide (1 h) followed by displacement with the appropriate amine would produce a compound (II) of Formula (I).

A corollary of Scheme Ito furnish useful intermediates is described in Scheme II below.

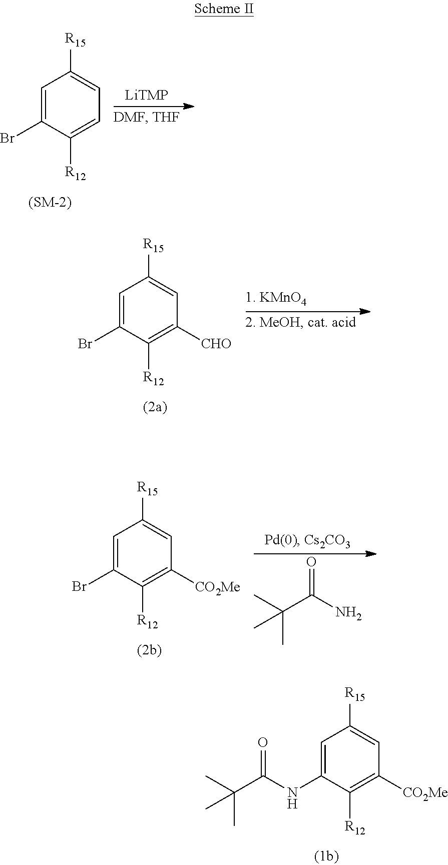

An appropriately substituted bromoarene (SM-2) can be deprotonated by a suitable strong base such as lithium 2,2,6,6-tetramethyl-piperinide and quenched with DMF to provide the corresponding aldehyde (2a). Oxidation to the acid and subsequent esterification would furnish the bromoester (2b). Palladium (0) mediated amidation with pivalamide would yield the amidoester (1b) which can be elaborated to compounds of Formula (I) via Scheme I.

Another alternative route for making compounds of the present invention is outlined below in Scheme III.

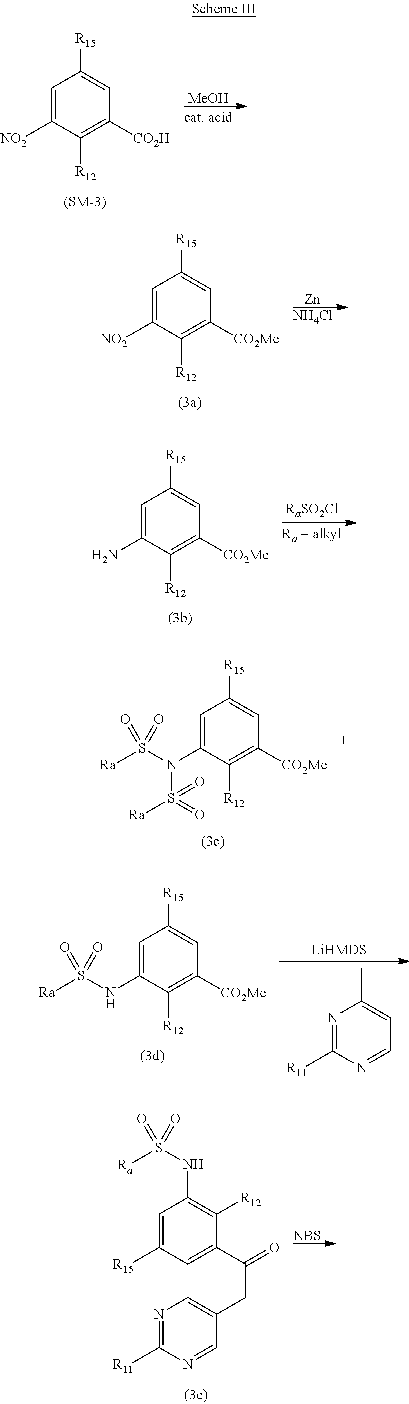

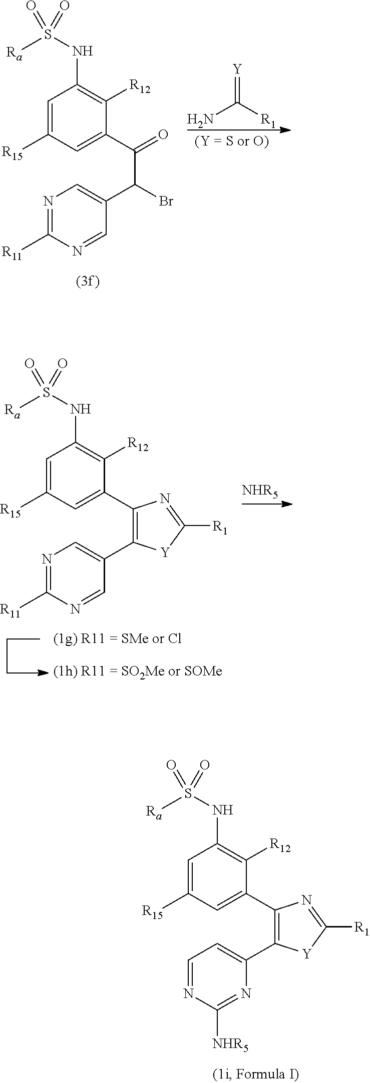

Simple nitrobenzoic acids (SM-3) can be easily converted to esters (3a). Nitro reduction with Zn-ammonium chloride or any other appropriate nitro reduction methodology can provide the useful anilinoester (3b). Treatment with an appropriate sulfonyl chloride can furnish the sulfonylimide (3c) or the corresponding sulfonylamide (3d). Deprotonation of 4-methyl-2-(methylthio)pyrimidine or 4-methyl-2-chloropyrimidine followed by addition to (3c) or (3d) could furnish the elaborated ketone (3e) with concomitant sulfonylimide cleavage. Treatment with N-bromosuccinimide (NBS) would then yield the bromoketone sulfonamide (31). Cyclocondensation with an appropriately substituted thioamide or amide would provide the corresponding thiazole or oxazole (1g) which can be elaborated to compounds of Formula (I) via the route summarized in Scheme I.

Yet another route to accessing compounds of the present invention is summarized in Scheme IV.

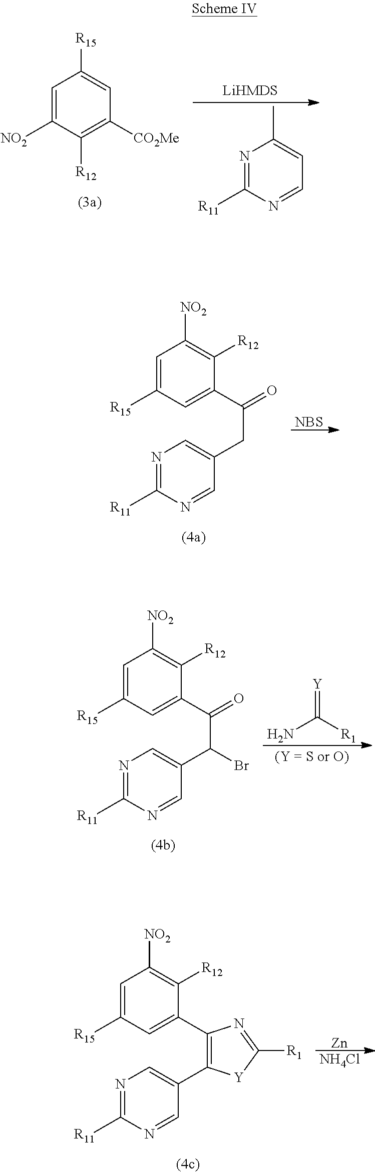

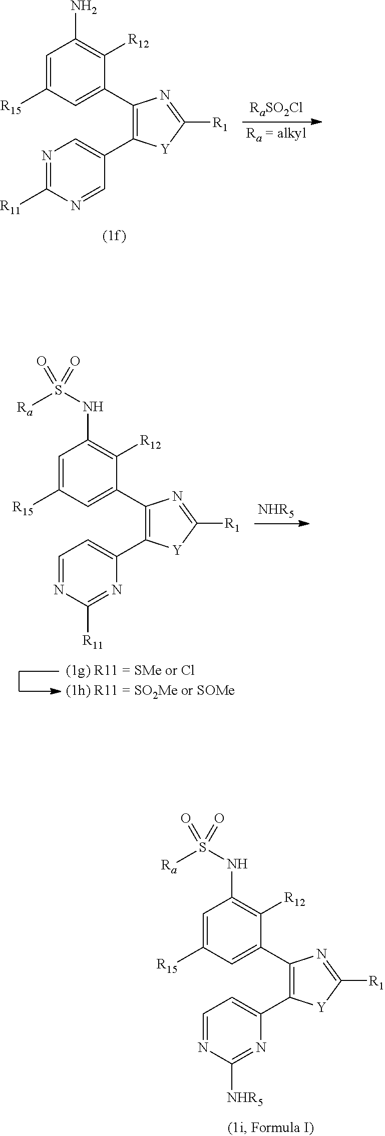

Deprotonation of 4-methyl-2-(methylthio)pyrimidine or 4-methyl-2-chloropyrimidine followed by addition to (3a) could furnish the elaborated ketone (4a). Treatment with N-bromosuccinimide (NBS) would then yield the bromoketone sulfonamide (4b). Cyclocondensation with an appropriately substituted thioamide or amide would then provide the corresponding thiazole or oxazole (4c). Nitro reduction with Zn/ammonium chloride or any other appropriate nitro reduction methodology would give the aniline (1f) which can be elaborated to compounds of Formula (I).

Compounds of Formula (I) can also be prepared by the procedures outlined below in Scheme V.

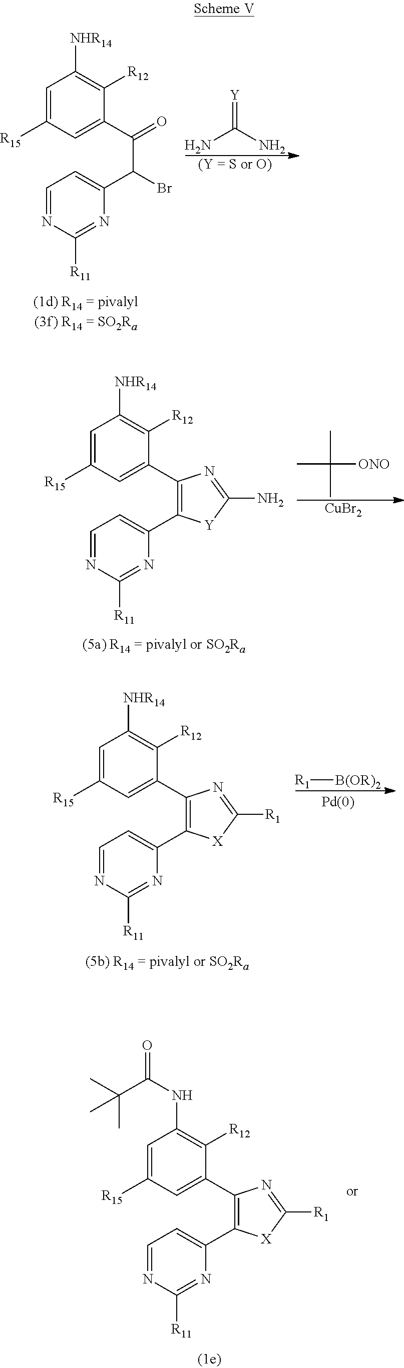

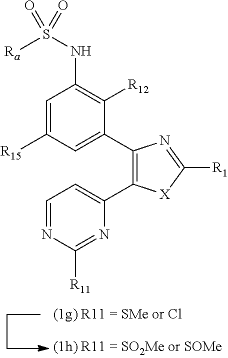

Cyclocondensation of (1d) or (3d) with urea or thiourea would yield the corresponding C-2 aminothiazole or aminooxazole (5a). A Sandmeyer reaction could provide the desired C-2 bromo heterocycle (5b). Suzuki cross-coupling with the desired boronate ester or acid affords the trisubstituted heterocycle (1e) or (1g) which then can be elaborated to compounds of Formula (I).

The compounds of the present invention (including intermediates) may be isolated and used per se or in the form of their pharmaceutically acceptable salts, solvates and/or hydrates. Many of the compounds represented by Formula I are capable of forming acid addition salts, particularly pharmaceutically acceptable acid addition salts. Pharmaceutically acceptable acid addition salts of the compound of the present invention include those of inorganic acids, for example, hydrohalic acids such as hydrochloric acid, hydrobromic acid, hydroiodic acid, nitric acid, sulfuric acid, phosphoric acid; and organic acids, for example aliphatic monocarboxylic acids such as formic acid, acetic acid, acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid. These salts may be prepared from compounds of Formula I by known salt-forming procedures.

Compounds of the present invention which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine. These salts may be prepared from compounds of Formula I by known salt-forming procedures.

For those compounds containing an asymmetric carbon atom, the compounds exist in individual optically active isomeric forms or as mixtures thereof, e.g. as racemic or diastereomeric mixtures. Unless specified otherwise, the present invention embraces both individual optically active R and S isomers as well as mixtures, e.g. racemic or diastereomeric mixtures, thereof. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereoisomers on the basis of their physical/chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a commercially available chiral High pressure liquid chromatography (HPLC) column.

The compounds of the present invention may exist in unsolvated as well as solvated forms with pharmaceutically acceptable solvents such as water, ethanol, and the like, and it is intended that the invention embrace both solvated and unsolvated forms. For purposes of the present invention, solvates (including hydrates) are considered pharmaceutical compositions, e.g., a compound of Formula I (or pharmaceutically acceptable salt thereof) in combination with an excipient, wherein the excipient is a solvent. The compound per se, pharmaceutical salt thereof, or a solvate/hydrate of the compound or salt may exist in either amorphous or crystalline form (e.g., polymorphs).

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

The present invention includes all pharmaceutically acceptable isotopically-labeled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸ iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labeled compounds of formula (I) can generally be prepared by conventional techniques known to those skilled in the art or by processes analogous to those described in the accompanying Examples and Preparations Sections using an appropriate isotopically-labeled reagent in place of the non-labeled reagent previously employed.

Please have some one from biology take a look at this section The compounds of the invention are useful in vitro and/or in vivo in inhibiting the growth of cancer cells. Consequently, the compounds of the present invention (including the compositions and processes used therein) may be used in the manufacture of a medicament for the therapeutic applications described herein. The compounds may be used alone or in compositions together with a pharmaceutically acceptable carrier, solvents (including water), or excipient. Suitable pharmaceutically acceptable carriers, diluents, or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference. The pharmaceutical compositions include the incorporation of solvents (including water) into a crystalline matrix of the compound (also referred to as solvates and hydrates).

Compounds of the invention modulate the activity of kinases and, as such, are useful for treating diseases or disorders in which kinases contribute to the pathology and/or symptomology of the disease. Examples of kinases that are inhibited by the compounds and compositions described herein and against which the methods described herein are useful include, but are not limited to, B-Raf, including mutant forms of B-Raf.

The mitogen-activated protein kinase (MAPK) pathway mediates the activity of a number of effector molecules which coordinate to control cellular proliferation, survival, differentiation and migration. Stimulation of cells by, for example, growth factors, cytokines or hormones results in the plasma membrane-associated Ras becoming GTP-bound and thereby activated to recruit Raf. This interaction induces the kinase activity of Raf leading to direct phosphorylation of MAPK/ERK (MEK), which in turn phosphorylates the extracellular signal-related kinase (ERK). Activated ERK then phosphorylates a wide array of effector molecules, for example, kinases, phosphatases, transcription factors and cytoskeletal proteins. Therefore, the Ras-Raf-MEK-ERK signaling pathway transmits signals from cell surface receptors to the nucleus and is essential, for example, in cell proliferation and survival. The regulation of this signaling cascade is further enriched by the multiple isoforms of Ras (including K-Ras, N-Ras and H-Ras), Raf (A-Raf, B-Raf, C-Raf/Raf-1), MEK (MEK-1 and MEK-2) and ERK (ERK-1 and ERK-2). Since 10-20% of human cancers harbor oncogenic Ras mutations and many human cancers have activated growth factor receptors, this pathway is an ideal target for intervention.

The essential role and the position of Raf in many signaling pathways has been demonstrated from studies using deregulated and dominant inhibitory Raf mutants in mammalian cells as well as from studies employing biochemical and genetic techniques to model organisms. In the past, the focus on Raf being an anti-tumor drug target centered on its function as a downstream effector of Ras. However, recent findings suggest that Raf may have a prominent role in the formation of certain tumors with no requirement of an oncogenic Ras allele. In particular, activating alleles of B-Raf and N-Ras have been identified in ˜70% of melanomas, 40% of papillary thyroid carcinoma, 30% of ovarian low-grade carcinoma, and 10% of colorectal cancers. Mutations in K-Ras occur in approximately 90% of pancreatic cancers. Most B-Raf mutations are found within the kinase domain, with a single substitution (V600E) accounting for at least 80%. The mutated B-Raf proteins activate the Raf-MEK-ERK pathway either via elevated kinase activity towards MEK or via activating C-Raf.

Therefore, development of a kinase inhibitor for B-Raf provides a new therapeutic opportunity for treatment of many types of human cancers, especially for metastatic melanomas, solid tumors, brain tumors such as Glioblastoma multiform (GBM), acute myelogenous leukemia (AML), lung cancer, papillary thyroid carcinoma, ovarian low-grade carcinoma, and colorectal cancer. Several Raf kinase inhibitors have been described as exhibiting efficacy in inhibiting tumor cell proliferation in vitro and/or in vivo assays (see, for example, U.S. Pat. Nos. 6,391,636, 6,358,932, 6,037,136, 5,717,100, 6,458,813, 6,204,467, and 6,268,391). Other patents and patent applications suggest the use of Raf kinase inhibitors for treating leukemia (see, for example, U.S. Pat. Nos. 6,268,391, 6,204,467, 6,756,410, and 6,281,193; and abandoned U.S. Patent Application Nos. 20020137774 and 20010006975), or for treating breast cancer (see, for example, U.S. Pat. Nos. 6,358,932, 5,717,100, 6,458,813, 6,268,391, 6,204,467 and 6,911,446). Data demonstrates that Raf kinase inhibitors can significantly inhibit signaling through the MAPK pathway, leading to dramatic shrinkage in B-Raf (V600E) tumors.

Some Raf inhibitors, in addition to increasing MEK and ERK signaling in wild-type B-Raf cells, also induce cell growth in cancer cell lines and cause transformation and growth in fibroblasts. The induction of downstream signaling has previously been attributed to published Raf pathway feedback loops. However, induction of pMEK and pERK can occur within minutes of Raf inhibitor treatment, even before reported feedback phosphorylation events are seen on B-Raf and C-Raf. The induction of signaling and cell growth both occur in a biphasic pattern, with low compound concentrations (0.01-0.1 μM) causing maximal induction, and higher compound concentrations (1-10 μM) causing less profound induction. Such a biphasic pattern is also observed in biochemical assays with purified wild-type B-Raf or C-Raf and is suggestive of a mechanism involving the interaction of two signaling subunits. In addition, Raf dimerization can up regulate pMEK, not through trans-phosphorylation of Raf molecules but presumably by a conformational activation of the kinase. Consistent with that model, Raf inhibitor treatment induces B-Raf/C-Raf dimer formation in cells. In addition, knockdown of A- or B-Raf with siRNA does not abrogate the Raf inhibitor induction of pMEK and pERK, and knockdown of C-Raf only slightly decreases the induction. Notably, knockdown of K-Ras in K-Ras mutant cells also only slightly decreases the induction, implying that this effect is not primarily mediated by Ras. Taken together, the data suggest a model in which inhibitor binding to one Raf molecule induces dimerization and conformational activation of a partner Raf molecule in the dimer. This can explain why wild-type Raf and mutant Ras tumors are insensitive to selective Raf kinase inhibitors and might also have important implications for toxicity, since induction of strong mitogenic signaling could lead to hyper proliferation of normal tissues. Understanding the Raf inhibitor induction mechanism may lead to the design of improved inhibitors.

The addition of a MEK inhibitor in combination with a Raf inhibitor leads to a significant inhibition of ERK signaling and consequently a decrease in cellular proliferation and transformation. Since MEK inhibitor treatments alone have lead to dose limiting toxicities in the clinic, a Raf plus MEK inhibitor combination represents a superior treatment strategy.

The compounds of the present invention inhibit cellular processes involving B-Raf kinase by blocking the signal cascade in these cancer cells and ultimately inducing stasis and/or death of the cells.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day. Suitable unit dosage forms for oral administration comprise from ca. 1 to 50 mg active ingredient.

The pharmaceutical formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

In some therapies, it may be advantageous to administer the compounds of the invention in combination with one or more therapeutic agents (pharmaceutical combinations). For example, synergistic effects can occur with other anti-tumor or anti-proliferative agents, for example, mitotic inhibitors, alkylating agents, anti-metabolites, intercalating antibiotics, growth factor inhibitors (e.g., trastuzumab, panitumumab, cetuximab, ipilimumab, tremelimumab, ramucirumab, gefitinib, erlotinib, lapatinib, sorafenib, dasatinib, sunitinib, dovitinib, etc.), cell cycle inhibitors, enzymes, topoisomerase inhibitors, biological response modifiers, antibodies, cytotoxics, anti-hormones, anti-androgens, an anti-angiogenesis agent, kinase inhibitor, pan kinase inhibitor or growth factor inhibitor. Suitable therapeutic agents include erlotinib, docetaxel, gemcitabine, cisplatin, carboplatin, paclitaxel, bevacizumab, trastuzumab, pertuzumab, temozolomide, tamoxifen, doxorubicin, rapamycin and lapatinib. Other suitable therapeutic agents are listed in the Physicians Desk Reference.

For example, the addition of a MEK inhibitor in combination with a Raf inhibitor leads to a significant inhibition of ERK signaling and consequently a decrease in cellular proliferation and transformation. Since MEK inhibitor treatments alone have lead to dose limiting toxicities in the clinic, a Raf plus MEK inhibitor combination represents a superior treatment strategy.

In another embodiment of the invention are combinations and methods of treating cancer comprising a therapeutically effective amount of a compound of the Summary of the Invention (Raf inhibitor) and at least one MEK protein kinase inhibitor. Preferred therapeutic agents for combination therapy include MEK inhibitors (e.g. AZD6244 (Example 10 of WO 03/077914), 2-(2-fluoro-4-iodophenylamino)-N-(2-hydroxyethoxy)-1,5-dimethyl-6-oxo-1,6-dihydropyridine-3-carboxamide, 4-(4-bromo-2-fluorophenylamino)-N-(2-hydroxyethoxy)-1,5-dimethyl-6-oxo-1,6-dihydropyridazine-3-carboxamide, PD-0325901(N-[(2-R)-2,3-dihydroxypropoxy]-3,4-difluoro-2-[(2-fluoro-4-iodophenyl)amino]-benzamide available from Axon Medchem), PD-184352(2-(2-chloro-4-iodophenyl)amino-N-(cyclopropylmethoxy)-3,4-difluorobenzamide available from Axon Medchem),

3,4-difluoro-2-(2-fluoro-4-iodophenylamino)-N-(2-hydroxyethoxy)-5-((3-oxomorpholino)methyl)benzamide CH-4987655 (Roche-Chugai) SL-327 (α-[amino[(4-aminophenyl)thio]methylene]-2-(trifluoromethyl)benzeneacetonitrile available from Axon Medchem), XL-518(Exelixis), AR-119(Ardea Biosciences, Valeant Pharmaceuticals), AS-701173(Merck Serono), AS-701255(Merck Serono), 360770-54-3(Wyeth), RDEA119 ((S)—N-(3,4-difluoro-2-(2-fluoro-4-iodophenylamino)-6-methoxyphenyl)-1-(2,3-dihydroxypropyl)cyclopropane-1-sulfonamide)); AS703026 (EMD Serono); MSC1936369B (EMD Serono); GSK1120212 (GlaxoSmithKline); ARRY-438162 (Array BioPharma); GDC0941 (Genentech); GDC0973 (Genentech); TAK-733 (Millennium Pharmaceuticals, Inc.); RO5126766 (Hoffmann-La Roche); and ARRY162 (Array Biopharma). mTOR inhibitors (e.g., Rapamycin (sirolimus), TORISEL™ (temsirolimus), RAD001 (everolimus), AP23573(deforolimus), OSI-027(OSI Pharmaceuticals), compounds described in WO 06/090167; WO 06/090169; WO 07/080,382, WO 07/060,404; and WO08/023,161): and

PI3K inhibitors (e.g., wortmannin, 17-hydroxywortmannin analogs described in WO 06/044453, 4-(2-(1H-indazol-4-yl)-6-(4-(methylsulfonyl)piperazin-1-yl)methyl)thieno-[3,2-d]pyrimidin-4-yl)morpholine, (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one, 4-(2-(1H-indazol-4-yl)-6-(4-(methylsulfonyl)piperazin-1-yl)methyl)thieno-[2,3-d]pyrimidin-4-yl)morpholine, LY294002(2-(4-Morpholinyl)-8-phenyl-4H-1-benzopyran-4-one available from Axon Medchem), PI 103 hydrochloride (3-[4-(4-Morpholinylpyrido[3′,′: 4,5]furo[3,2-d]pyrimidin-2-yl]phenol hydrochloride available from Axon Medchem), PIK 75 (N′-[(1E)-(6-bromoimidazo[1,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydro-imidazo[1,2-c]quinazolin-5-yl)-nicotinamide available from Axon Medchem), GDC-0941 bismesylate (-(1H-Indazol-4-yl)-6-(4-methanesulfonyl-piperazin-1ylmethyl)-4-morpholin-4-yl-thieno[3,2-d]pyrimidine bismesylate available from Axon Medchem), PKI587 1-(4-(4-(dimethylamino)piperidine-1-carbonyl)phenyl)-3-(4-(4,6-dimorpholino-1,3,5-triazin-2-yl)phenyl)urea (Wyeth), 2126458 2,4-difluoro-N-(2-methoxy-5-(4-(pyridazin-4-yl)quinolin-6-yl)pyridin-3-yl)benzenesulfonamide (GSK), PF-04691502 2-amino-8-((1r,4r)-4-(2-hydroxyethoxy)cyclohexyl)-6-(6-methoxypyridin-3-yl)-4-methylpyrido[2,3-d]pyrimidin-7(8H)-one (Pfizer), BEZ235 (2-Methyl-2-[4-(3-methyl-2-oxo-8-quinolin-3-yl-2,3-dihydro-imidazo[4,5-c]quinolin-1-yl)-phenyl]-propionitrile available from Novartis), BKM120 (5-(2,6-dimorpholinopyrimidin-4-yl)-4-(trifluoromethyl)pyridin-2-amine (Novartis), AS-252424 (5-[1-[5-(4-Fluoro-2-hydroxy-phenyl)-furan-2-yl]-meth-(Z)-ylidene]-thiazolidine-2,4-dione available from Axon Medchem), TGX-221 (7-Methyl-2-(4-morpholinyl)-9-[1-(phenylamino)ethyl]-4H-pyrido-[1,2-a]pyrimidin-4-one available from Axon Medchem), XL-765, and XL-147 (Exelixis).

Compounds of the invention, when administered in conjunction with other therapies, dosages of the co-administered compounds will of course vary depending on the type of co-drug employed, on the specific drug employed, on the condition being treated and so forth.

According to the methods of the invention, a compound of the present invention or a combination of a compound of the present invention and at least one additional pharmaceutical agent is administered to a subject in need of such treatment, preferably in the form of a pharmaceutical composition. In the combination aspect of the invention, the compound of the present invention and at least one other pharmaceutical agent (described above) may be administered either separately or in the pharmaceutical composition comprising both. It is generally preferred that such administration be oral. However, if the subject being treated is unable to swallow, or oral administration is otherwise impaired or undesirable, parenteral or transdermal administration may be appropriate.

According to the methods of the invention, when a combination of a compound of the present invention and at least one other pharmaceutical agent are administered together, such administration can be sequential in time or simultaneous with the simultaneous method being generally preferred. For sequential administration, a compound of the present invention and the additional pharmaceutical agent can be administered in any order. It is generally preferred that such administration be oral. It is especially preferred that such administration be oral and simultaneous. When a compound of the present invention and the additional pharmaceutical agent are administered sequentially, the administration of each can be by the same or by different methods.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The invention also provides for a pharmaceutical combinations, e.g. a kit, comprising a) a first agent which is a compound of the invention as disclosed herein, in free form or in pharmaceutically acceptable salt form, and b) at least one additional therapeutic agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### EXAMPLES

The present invention is further exemplified, but not limited, by the following intermediates and examples that illustrate the preparation of compounds of the present invention.

Preparative separations are carried out using a CombiFlash® Rf system (Teledyne Isco Inc. Lincoln, Nebr.) in combination with RediSep® Normal-Phase Silica Flash Columns (4 g-120 g, 35-70 micron particle size; Teledyne Isco Inc.), or by flash column chromatography using silica gel (230-400 mesh) packing material, or by HPLC using a WATERS 2767 Sample Manager, C-18 reversed phase column, 30×50 mm, flow 75 mL/min. Typical solvents employed for the CombiFlash® system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid (TFA).

Microwave reactions are conducted in a Creator or Initiator microwave system (Biotage, Charlottesville, Va.)

The following acronyms having the corresponding meanings are used in the experimental section below.

DEAD—diethyl azodicarboxylate

DIEA—diisopropylethyl amine

THF—tetrahydrofuran

DCM—ichloromethane

DMF—dimethylformamide

HOAc—acetic acid

DME—1,1-dimethoxyethane

ACN—acetonitrile

EtOAc—ethyl acetate

NMP—N-methylpyrrolidinone

mCPBA—m-chloroperbenzoic acid

TFA—trifluoroacetic acid

LiHMDS—lithium bis(trimethylsilyl)amide

MeOH—methanol

dba—dibenzylideneacetone

Et₂O—diethyl ether

NBS—N-bromosuccinimide

DMSO—dimethyl sulfoxide

rt—room temperature

TLC—thin layer chromatography

### Preparation of Key Starting Materials and Intermediates

### Preparation of starting material methyl-2-chloro-5-fluoro-3-pivalamidobenzoate (I, Scheme I, (1b))

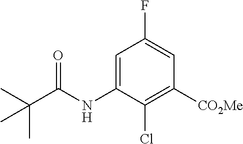 [SMILES: COC(=O)c1cc(F)cc(NC(=O)C(C)(C)C)c1Cl]

### Step 1. Preparation of N-(3-bromo-2-chloro-5-fluorphenyl)pivalamide

To a round bottom flask under nitrogen fitted with a stir bar was added 1-chloro-2,6-dibromo-4-fluorobenzene (14.36 g, 49.8 mmol), pivalamide (5.04 g, 49.8 mmol), cesium carbonate (21.09 g, 64.7 mmol) and dioxane (250 mL). The reaction mixture was sparged with nitrogen and Pd(dba)₂ (1.43 g, 2.490 mmol) and 5-bis(diphenylphosphino)-9,9-dimethylxanthene (XANTPHOS, 2.02 g, 3.49 mmol) were added. The reaction was then sealed and heated in an oil bath at 70° C. for 18 h. The reaction was allowed to cool to r.t and was partitioned between a saturated aqueous solution of NH₄Cl and EtOAc. The layers were separated and the aqueous portion was extracted with EtOAc (2×). The combined organic portions were washed with water, brine, dried (Na₂SO₄), filtered, concentrated, and adsorbed onto silica gel. Purification by flash chromatography on silica gel using an EtOAc-heptane (1-20%) elution gradient afforded N-(3-bromo-2-chloro-5-fluorphenyl)pivalamide (11.0 g, 33.8 mmol, 68%) as a white crystalline solid. LCMS (m/z): 309.9 (MH⁺), t_{R}=1.11

### Step 2. Preparation of methyl-2-chloro-5-fluoro-3-pivalamidobenzoate

To a steel pressure reaction vessel fitted with a stir bar was added N-(3-bromo-2-chloro-5-fluorophenyl)pivalamide (6.22 g, 20.16 mmol), MeOH (100 mL), triethylamine (5.62 mL, 40.3 mmol). The resulting solution was sparged with nitrogen for 5 min, then [(R)-(+)-2,2′-bis(diphenylphosphino)-1,1′-binaphthyl]palladium(II) chloride (0.323 g, 0.403 mmol) was added. The reaction vessel was sealed and pressurized with carbon monoxide (70 psi). The reaction was then placed in an oil bath and heated to 100° C. for 18 h. The reaction mixture was allowed to cool to rt, diluted with water and extracted twice with EtOAc. The organics were combined, washed with brine, dried (Na₂SO₄), filtered and concentrated. The resulting red oil was adsorbed onto silica gel and purified by flash chromatography on silica gel eluting with an EtOAc-heptane (0-20%) gradient. Product fractions were combined and concentrated to afford methyl-2-chloro-5-fluoro-3-pivalamidobenzoate (3.30 g, 11.36 mmol, 56%) as a white solid. LCMS (m/z): 288.0 (MH⁺), t_{R}=1.03 min

### Preparation of starting material methyl-5-chloro-2-fluoro-3-pivalamidobenzoate (II, Scheme II, (1b))

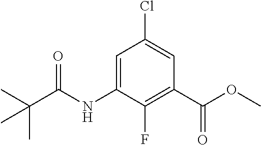 [SMILES: COC(=O)c1cc(Cl)cc(NC(=O)C(C)(C)C)c1F]

### Step 1. Preparation of 3-bromo-5-chloro-2-fluorobenzaldehyde

A solution of n-BuLi/hexane (2.0 M, 24.3 mL, 48.6 mmol) was added to a cooled solution of 2,2,6,6-tetramethyl-piperidine (6.86 g, 48.6 mmol) in dry THF (48 mL) at −75° C. over 15 min while maintaining an internal temperature between −75 to −67° C. After addition, the reaction was maintained between −70 to −67° C. for 30 min. 2-Bromo-4-chloro-1-fluorobenzene (5.2 mL, 9.0 g, 43.0 mmol) was added over 10 min while maintaining the internal temperature between −70 to −67° C. The resulting reaction was maintained at this temperature for 40 min. DMF (4.4 ml, 56.7 mmol) was then added dropwise over 15 min while maintaining the internal temperature between at −70 to −65° C., After 1 h, TLC indicated complete conversion and the reaction was quenched with saturated aqueous NH₄Cl solution (15 mL) between −60 to −30° C. The resulting mixture was adjusted to pH 1-2 with aqueous 6.0 N HCl solution (25 mL) at −30 to 10° C. and partitioned between heptane (60 mL) and H₂O (15 mL). The layers were separated and the aqueous portion was extracted with heptane (50 mL). The combined organic portions were washed with brine (2×50 mL), dried (Na₂SO₄), and concentrated. The resulting crude material was purified by flash chromatography on silica gel using an EtOAc-heptane (0-20%) elution gradient to provide 3-bromo-5-chloro-2-fluorobenzaldehyde (10 g, 80% yield) as a light yellow solid: ¹H NMR (400 MHz, CDCl₃) δ ppm 7.74-7.85 (m, 2H) 10.29 (s, 1H).

### Step 2. Preparation of 3-bromo-5-chloro-2-fluorobenzoic acid

A suspension of 3-bromo-5-chloro-2-fluorobenzaldehyde (13.0 g, 54.7 mmol) in mixture of t-butanol (50 mL) and water (50 mL) was heated to 30° C., followed by portionwise addition of KMnO₄ over 25 min with an internal temperature between 30-45° C. The resulting mixture was then sequentially stirred at 45° C. for an additional 30 min, at 50-55° C. for 30 min and at 55-65° C. for 1.5 h. The reaction was then allowed to cool to rt, quenched with saturated aqueous Na₂SO₃ solution to a negative peroxide test, diluted with water (70 mL) and basified with saturated aqueous Na₂CO₃ solution (9 mL) and stirred for 10 min. The resulting mixture was filtered through a pad of Celite under reduced pressure and the filter cake was washed with water (2×50 mL). The combined filtrates were acidified with concentrated HCl to pH 1 at 15-25° C., and then extracted with EtOAc (2×100 mL). The combined organic extracts were sequentially washed with water (100 mL), brine (100 mL), dried (Na₂SO₄), and concentrated to give 3-bromo-5-chloro-2-fluorobenzoic acid (11.0 g, 79% yield) as a light yellow solid: ¹H NMR (300 MHz, DMSO-d⁶) δ ppm 7.76-7.92 (m, 1H) 8.07-8.28 (m, 1H) 13.37-14.02 (m, 1H).

### Step 3. Preparation of methyl 3-bromo-5-chloro-2-fluorobenzoate

A solution of 3-bromo-5-chloro-2-fluorobenzoic acid (7.5 g, 29.6 mmol) in methanol (100 mL, 2470 mmol) was treated with conc. H₂SO₄ (8 mL, 29.6 mmol) and the resulting mixture was heated to reflux overnight. The reaction mixture was allowed to cool to rt, diluted with ice water (200 mL), and extracted with EtOAc (2×200 mL). The combined organic extracts were washed with saturated aqueous Na₂CO₃ solution (2×100 mL), brine (100 mL), dried (Na₂SO₄), and concentrated to furnish methyl 3-bromo-5-chloro-2-fluorobenzoate (7.2 g, 26.9 mmol, 91% yield) as a pale yellow solid: ¹H NMR (400 MHz, CDCl₃) δ ppm 3.95 (s, 3H) 7.69-7.77 (m, 1H) 7.86 (dd, J=5.5, 2.4Hz, 1H).

### Step 4. Preparation of methyl-5-chloro-2-fluoro-3-pivalamidobenzoate

To a sealed tube fitted with a stir bar was added methyl 3-bromo-5-chloro-2-fluorobenzoate (7.39 g, 27.6 mmol), pivalamide (8.38 g, 83 mmol), cesium carbonate (11.70 g, 35.9 mmol) and 1,4-dioxane (30 mL). The resulting mixture was purged with Ar for 5 min followed by the addition of Pd(dba)₂ (0.79 g, 1.38 mmol) and XANTPHOS (1.12 g, 1.93 mmol) followed by another 5 min sparge with Ar, The reaction tube was sealed and heated in oil bath at 110° C. for 3 h. LCMS of an aliquot indicated complete conversion and the reaction was allowed to combined organic portions were sequentially washed with water, brine, dried (Na₂SO₄), and concentrated. The resulting material was adsorbed onto silica gel. and purified by flash chromatography on silica gel using an EtOAc-heptane (1-10%) elution gradient to provide methyl-5-chloro-2-fluoro-3-pivalamidobenzoate (4.76 g, 16.5 mmol, 60% yield) as a yellow solid: LCMS (m/z): 288.0 (MH⁺), t_{R}=0.96 min

### Preparation of starting material methyl 2-fluoro-3-(N-(propylsulfonyl)propylsulfonamido)benzoate (III, Scheme III, (3c)

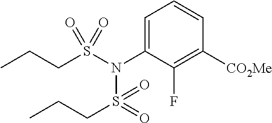 [SMILES: CCCS(=O)(=O)N(c1cccc(C(=O)OC)c1F)S(=O)(=O)CCC]

### Step 1: Preparation of methyl 2-fluoro-3-nitrobenzoate

A solution of 2-fluoro-3-nitrobenzoic acid (5 g, 27.0 mmol) in MeOH (50 mL) was treated with concentrated H₂SO₄ (1.4 mL, 27.0 mmol) and the resulting reaction was stirred at 50° C. for 16 h. The solvent was removed under reduced pressure and the residue was diluted with EtOAc. The organic solution was washed with saturated aqueous NaHCO₃ solution until the water phase reached neutral pH. The two phases were separated. Organics were washed with water, brine and dried (Na₂SO₄). The solution was filtered and concentrated to give crude methyl 2-fluoro-3-nitrobenzoate (5.1 g, 25.2 mmol, 93%) as a pale yellow solid which was used as is in the next step.

LCMS (m/z): 258.1 (MH⁺), t_{R}=0.74 min; ¹H NMR (400 MHz, CDCl₃,): δ ppm 4.00 (s, 3H), 7.38 (t, J=8.0 Hz, 1H), 8.14-8.27 (m, 2H).

### Step 2: Preparation of methyl 3-amino-2-fluorobenzoate

To a solution of 2-fluoro-3-nitrobenzoate (2.6 g, 12.8 mmol) in MeOH (150 mL) was added zinc dust (8.4 g, 128 mmol) and the mixture was cooled to 0° C. in an ice bath. To this suspension ammonium chloride (6.9 g, 128 mmol) was added in portions over 10 min. The heterogenous reaction mixture was allowed to warm to rt and stirred for 1 h. The reaction mixture was filtered through a pad of Celite and the collected filtrate was concentrated to an off white solid. This residue was suspended in EtOAc, sonicated, and filtered through Celite and the resulting filtrate concentrated to yield methyl 3-amino-2-fluorobenzoate (2.04 g, 11.8 mmol, 92%) as a brown oil. This material was carried on to the next step without further purification.

LCMS (m/z): 170.0 (MH⁺), 211.1 (M+ACN), t_{R}=0.51 min; ¹H NMR (CDCl₃, 400 MHz): 6 ppm 3.85 (br. s., 2H), 3.92 (s, 3H), 6.87-7.03 (m, 2H), 7.19-7.33 (m, 2H).

### Step 3: Methyl 2-fluoro-3-(N-(propylsulfonyl)propylsulfonamido)benzoate

Methyl 3-amino-2-fluorobenzoate (2.04 g, 12.1 mmol) was dissolved in DCM (100 mL) in a round bottom flask equipped with a magnetic stirbar. Et₃N (5.0 mL, 36.2 mmol) was added via a syringe. The reaction mixture was cooled to 0° C. and propane-1-sulfonyl chloride (1.6 mL, 14.3 mmol) was added dropwise. The clear orange reaction mixture was stirred at rt overnight. LCMS indicated that the reaction was only partially complete, additional propane-1-sulfonyl chloride (1.4 mL, 12.1 mmol) was then added, and the reaction was stirred for 3 h. A second LCMS analysis revealed some starting material remaining. Propane-1-sulfonyl chloride (0.14 mL, 1.2 mmol) was added again, and the reaction mixture stirred for another 2 h. The reaction mixture was then diluted with DCM and quenched with water. The two phases were separated, the organic portion was washed with water, brine and dried (Na₂SO₄). Evaporation of the solvent afforded 4.77 g of crude material. Purification by flash column chromatography on silica gel (0-50% EtOAc-heptane) followed by trituration with Et₂O provided methyl 2-fluoro-3-(N-(propylsulfonyl)propylsulfonamido)benzoate (3.41 g, 8.94 mmol, 74%) as a pale orange solid.

LCMS (m/z): 382.2 (MH⁺), t_{R}=0.95 min; ¹H NMR (CDCl₃, 400 MHz): δ ppm 1.10 (t, J=7.4Hz, 6H), 1.90-2.04 (m, 4H), 3.54 (m, 2H), 3.66 (m, 2H), 3.96 (m, 3H), 7.31 (t, J=7.4Hz, 1H), 7.58 (td, J=7.2, 2.0 Hz, 1H), 8.08 (td, J=7.2, 2.0 Hz, 4H).

### Preparation of starting material ethyl 2-chloro-3-(methylsulfonamido)benzoate (IV, Scheme III, (3d))

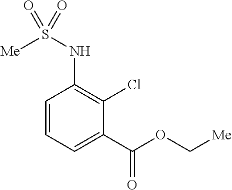 [SMILES: CCOC(=O)c1cccc(NS(C)(=O)=O)c1Cl]

### Step 1. Preparation of ethyl 2-chloro-3-nitrobenzoate

To a mixture at 0° C. of 2-chloro-3-nitrobenzoic acid (2.0 g, 9.9 mmol) in DCM, oxalyl chloride (1.0 mL, 11.9 mmol) was added, followed by addition of catalytic DMF (0.15 mL, 2.0 mmol). The resulting reaction was stirred for 15 min at which time the ice bath was removed and the reaction was allowed to warm to rt. The reaction was cooled to 0° C. again and EtOH (12 mL, 200 mmol) was added. The reaction was maintained over 2 d, allowing the reaction to warm to rt. The reaction was concentrated in vacuo, and the resulting oil was dissolved in Et₂O and was washed with aqueous 1 M NaOH solution (25 mL), water (3×25 mL), and brine (25 mL). The Et₂O layer was dried (MgSO₄), filtered and stripped to afford ethyl 2-chloro-3-nitrobenzoate (2.0 g, 8.7 mmol, 88%) as a yellow oil.

¹H NMR (300 MHz, CDCl₃) δ ppm 1.42 (t, J=7.2 Hz, 3H) 4.45 (q, J=7.0 Hz, 2H) 7.48 (t, J=7.9 Hz, 1H) 7.84 (dd, J=8.1, 1.6 Hz, 1H) 7.94 (dd, J=7.8, 1.6 Hz, 1H)

### Step 2. Preparation of ethyl 3-amino-2-chlorobenzoate

To a solution of ethyl 2-chloro-3-nitrobenzoate (2 g, 8.71 mmol) in HOAc (50 mL) at 22° C., iron (4.86 g, 87 mmol) was added. The reaction produced an exotherm 15 min after addition. The reaction was allowed to stir for 18 h at rt. The reaction mixture was diluted with EtOAc and filtered through Celite, washing the collected solids thoroughly with EtOAc. The combined filtrates were washed with aqueous 1 M NaOH solution (2×200 mL), water (3×200 mL), and brine (200 mL). The EtOAc layer was dried (MgSO₄), filtered and concentrated to afford ethyl 3-amino-2-chlorobenzoate (1.69 g, 8.5 mmol, 97%) as a yellow oil: ¹H NMR (400 MHz, CDCl₃) δ ppm 1.40 (t, J=7.0 Hz, 3H) 4.24 (br. s., 2H) 4.39 (q, J=7.0 Hz, 2H) 6.88 (dd, J=7.8, 1.57 Hz, 1H) 7.05-7.19 (m, 2H)

### Step 3. Preparation of ethyl 2-chloro-3-(methylsulfonamido)benzoate

To a solution of ethyl 3-amino-2-chlorobenzoate (845 mg, 4.23 mmol) and pyridine (1.03 ml, 12.7 mmol) in DCM (4 mL), methanesulfonylchloride (0.33 mL, 4.2 mmol) was added and the reaction mixture was allowed to stir overnight. A TLC of the reaction indicated complete reaction. The reaction was concentrated to an oil and partitioned between EtOAc and water. The EtOAc layer was washed with water (3×25 mL) and brine (25 mL). The EtOAc layer was dried (MgSO₄), filtered and concentrated to afford a beige solid. The solid was dissolved in DCM and adsorbed onto silica gel. The material was purified by flash chromatography on silica gel using an EtOAc-heptane (0-100%) elution gradient to afford ethyl 2-chloro-3-(methylsulfonamido)benzoate (880 mg, 3.17 mmol, 74.9%) as a white solid. ¹H NMR (400 MHz, CDCl₃) δ ppm 1.41 (t, J=7.0 Hz, 3H) 3.01 (s, 3H) 4.42 (q, J=7.3 Hz, 2H) 7.01 (br. s., 1H) 7.37 (t, J=8.0 Hz, 1H) 7.64 (d, J=7.8 Hz, 1H) 7.83 (d, J=8.2 Hz, 1H)

### Preparation of starting material ethyl 2-chloro-3-(propylsulfonamido)benzoate (V, Scheme I, (1b))

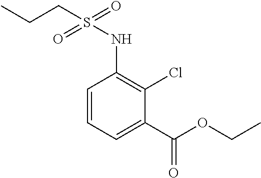 [SMILES: CCCS(=O)(=O)Nc1cccc(C(=O)OCC)c1Cl]

The compound was prepared using a similar procedure for starting material IV, step 3, substituting for the appropriate reagent.

LCMS (m/z): 306.0 (MH⁺), t_{R}=0.83 min; ¹H NMR (400 MHz, CDCl₃) δ ppm 1.03 (t, J=7.43 Hz, 3H) 1.41 (t, J=7.24Hz, 3H) 1.80-1.92 (m, 2H) J=7.83, 1.57 Hz, 1H) 7.85 (dd, J=8.22, 1.57 Hz, 1H)

### Preparation of starting material methyl 2-chloro-3-pivalamidobenzoate (VI, Scheme I, (1b))

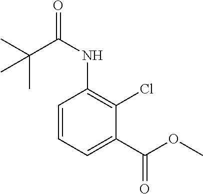 [SMILES: COC(=O)c1cccc(NC(=O)C(C)(C)C)c1Cl]

Steps 1 and 2 are the same as for intermediate (IV).

### Step 3. Preparation of methyl 2-chloro-3-pivalamidobenzoate

To a solution of methyl 3-amino-2-chlorobenzoate (2.5 g, 13.5 mmol) and Et₃N (3.8 ml, 26.9 mmol) in DCM (50 ml) at 0° C., pivaloyl chloride (1.8 ml, 14.8 mmol) was added. The reaction was allowed to warm to rt overnight. The mixture was diluted with EtOAc. The EtOAc layer was washed with, aqueous 1.0 M HCl solution, water, saturated aqueous sodium bicarbonate solution, water, brine, and dried (MgSO₄). The solution was filtered and concentrated to afford methyl 2-chloro-3-pivalamidobenzoate (3.5 g, 13.0 mmol, 96% yield) as a pale pink oil which was used without further purification:

¹H NMR (400 MHz, CDCl₃) δ ppm 1.36 (s, 9H) 3.94 (s, 3H) 7.33 (t, J=8.0 Hz, 1H) 7.54 (dd, J=7.8, 1.57 Hz, 1H) 8.24 (br. s., 1H) 8.60 (dd, 1H).

### Preparation of starting material ethyl 2,5-dichloro-3-pivalamidobenzoate (VII Scheme, I, (1b))

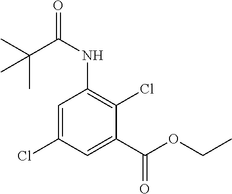 [SMILES: CCOC(=O)c1cc(Cl)cc(NC(=O)C(C)(C)C)c1Cl]

### Step 1: Preparation of ethyl 3-amino-2,5-dichlorobenzoate

A solution of ethyl 2,5-dichloro-3-nitrobenzoate (2 g, 7.57 mmol, prepared following the procedure for starting material I, step 1) in MeOH (40 mL) was added to a mixture of iron powder (2.12 g, 37.9 mmol) and ammonium chloride (1.22 g, 22.7 mmol) in water (20 mL). The resulting suspension was stirred at 60° C. in an oil bath for 2 h under nitrogen. The suspension was cooled to rt and diluted with EtOAc. The organic solution was washed with water, brine, dried (Na₂SO₄), and concentrated to give the desired ethyl 3-amino-2,5-dichlorobenzoate (1.6 g, 90%) as yellow solid which was used in the next step without further purification: LCMS (m/z): 275 (MH⁺+ACN), t_{R}=0.91 min; ¹H NMR (400 MHz, DMSO-d⁶) δ ppm 1.28 (t, J=7.0 Hz, 3H), 4.27 (q, J=7.0 Hz, 2H), 5.94 (s, 2H), 6.83 (d, J=2.4Hz, 1H), 6.95 (d, J=2.4Hz, 1H).

### Step 2: Preparation of 2,5-dichloro-3-pivalamidobenzoate

A solution of 3-amino-2,5-dichlorobenzoate (1.6 g, 6.8 mmol) and triethylamine (1.9 mL, 13.7 mmol) in DCM under nitrogen was cooled to 0 C with an ice bath. Pivaloyl chloride (0.92 mL, 7.52 mmol) was added dropwise via a syringe. The reaction mixture was allowed to warm to room temperature and stirred for 3 h. LCMS showed 80% conversion, and additional triethylamine (1.0 mL, 6.9 mmol), and pivaloyl chloride (90 μL, 0.8 mmol) were added. Stirring was continued at room temperature for 15 h. The reaction mixture was diluted with EtOAc and the organics were washed with water, aqueous 1 N HCl solution, saturated aqueous sodium bicarbonate solution, brine, dried (Na₂SO₄) and concentrated to give 2,5-dichloro-3-pivalamidobenzoate (2.5 g, 84%) as a brown oil which was carried forward without further purification: LCMS (m/z): 318 (MH⁺), t_{R}=1.11 min.

### Preparation of starting material ethyl 2,5-dichloro-3-(N-(methylsulfonyl)methylsulfonamido)benzoate (VIII, Scheme III, (3c))

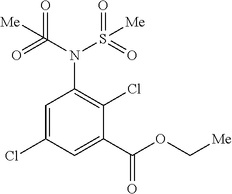

Prepared from ethyl 3-amino-2,5-dichlorobenzoate (intermediate VII, step 1), following a similar sulfonylation procedure as in intermediate IV, step 3: ¹H NMR (400 MHz, CDCl₃) δ ppm: 1.35 (t, 3H), 3.44 (s, 6H), 4.37 (q, 2H), 7.45 (s, 1H), 7.83 (s, 1H)

### Preparation of starting material ethyl ethyl 2,5-dichloro-3-(propylsulfonamido)benzoate (IX, Scheme III, (3d))

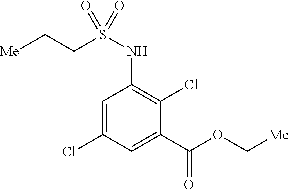 [SMILES: CCCS(=O)(=O)Nc1cc(Cl)cc(C(=O)OCC)c1Cl]

Prepared from ethyl 3-amino-2,5-dichlorobenzoate (intermediate VII, step 1), following the same sulfonylation procedure as in intermediate IV, step 3:

¹H NMR (400 MHz, CDCl₃) δ ppm: 0.98 (t, 3H), 1.34 (t, 3H), 1.79 (m, 2H) 3.03 (t, 2H), 4.34 (q, 2H), 6.88 (s, 1H), 7.50 (s, 1H), 7.79 (s, 1H).

### Preparation of starting material (5)-tert-butyl 1-aminopropan-2-ylcarbamate (X, NHR₅)

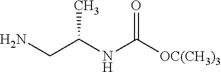

### Step 1. Preparation of (S)-tert-butyl 1-(1,3-dioxxisoindolin-2-yl)propan-2-ylcarbamate

To a stirred solution of (S)-tert-butyl 1-hydroxypropan-2-ylcarbamate (7.4 g, 42.2 mmol) in dry THF (420 mL) were added phthalimide (6.83 g, 46.4 mmol) and PPh₃ (12.2 g, 46.4 mmol). DEAD (7.3 mL, 46.4 mmol) was then added dropwise to the stirred solution at room temperature, and maintained for 3 h. The reaction mixture was then concentrated and the residue was purified by flash chromatography (SiO₂, 70:30-50:50 hexanes-EtOAc) to provide 12.5 g of (S)-tert-butyl 1-(1,3-dioxxisoindolin-2-yl)propan-2-ylcarbamate. LCMS (m/z): 205.1 (M+H-Boc), t_{R}=0.86 min; ¹H NMR (CDCl₃, 400 MHz) δ 7.82-7.87 (m, 2H), 7.67-7.75 (m, 2H), 4.60-4.76 (br d, 1H), 4.03-4.20 (br s, 1H), 3.62-3.72 (m, 2H), 1.25 (s, 9H), 1.21 (d, J=6.6 Hz, 3H).

### Step 2. Preparation of (S)-tert-butyl 1-aminopropan-2-ylcarbamate

Hydrazine monohydrate (20 mL, 643 mmol) was added to a suspension of (S)-tert-butyl 1-(1,3-dioxxisoindolin-2-yl)propan-2-ylcarbamate (12.5 g, 41.1 mmol) in dry MeOH (150 mL), and the resulting mixture was heated to 50° C. for 1 h. After cooling to room temperature, the reaction mixture was filtered through a sintered funnel, and the filtrate concentrated. The resulting residue was suspended in Et₂O (300 mL) and filtered, washing the filter cake thoroughly with Et₂O. The combined filtrates were filtered and concentrated to furnish 6.3 g of (S)-tert-butyl-1-aminopropan-2-ylcarbamate: ¹H NMR (CDCl₃, 400 MHz) δ 4.44-4.71 (br s, 1H), 3.53-3.74 (br m, 1H), 2.75 (dd, J=4.9, 12.9 Hz, 1H), 2.64 (dd, J=6.6, 12.9 Hz, 1H), 1.45 (s, 9H), 1.21 (d, J=6.6 Hz, 3H), 1.15-1.34 (br s, 2H), 1.12 (d, J=6.7 Hz, 3H).

### Preparation of starting material N-(3-(2-bromo-2-(2-(methylthio)pyrimidin-4-yl)acetyl)-2-chloro-5-fluorophenyl)pivalamide (XI, Scheme I, (1d))

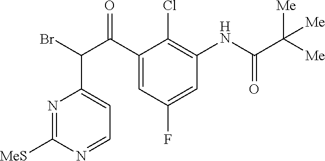 [SMILES: CSc1nccc(C(Br)C(=O)c2cc(F)cc(NC(=O)C(C)(C)C)c2Cl)n1]

### Step 1. Preparation of N-(2-chloro-5-fluoro-3-(2-(2-(methylthio)pyrimidin-4-yl)acetyl)phenyl)pivalamide

To a cooled solution of 4-methyl-2-(methylthio)pyrimidine (1.26 mL, 9.04 mmol) and methyl 2-chloro-5-fluoro-3-pivalamido-benzoate (I, 2.0 g, 6.95 mmol) in THF (35 mL) at 0° C., LiHMDS (1.0 M in THF, 25.7 mL, 25.7 mmol) was slowly added. The reaction was maintained at 0° C. for 2 h. LCMS of reaction aliquot indicated complete conversion and the reaction was quenched by the addition of 1.0 M aqueous HCl solution. The resulting mixture was stirred for 1 h at rt, whereupon EtOAc was then added and the resulting biphasic mixture was neutralized to pH 8 with saturated aqueous NaHCO₃ solution. The two phases were separated and aqueous phase was extracted with EtOAc. The organic phases were combined, washed with brine, dried (Na₂SO₄), and concentrated. The resulting residue was adsorbed onto silica gel and was purified by flash chromatography on silica gel using an EtOAc-heptane (0-30%) elution gradient to give N-(2-chloro-5-fluoro-3-(2-(2-(methylthio)pyrimidin-4-yl)acetyl)phenyl)pivalamide (2.24 g, 5.66 mmol, 81% yield) as a yellow solid: ¹H NMR (300 MHz, CDCl₃) δ ppm 1.37 (s, 9H) 2.62 (s, 3H) 5.70 (s, 1H) 6.64 (d, J=5.6 Hz, 1H) 7.03 (dd, J=8.2, 2.3 Hz, 1H) 8.27 (br. s., 1H) 8.36 (d, J=5.3 Hz, 1H) 8.41 (dd, J=10.6, 2.93 Hz, 1H).

### Step 2. Preparation of N-(3-(2-bromo-2-(2-(methylthio)pyrimidin-4-yl)acetyl)-2-chloro-5-fluorophenyl)pivalamide

To a solution of N-(2-chloro-5-fluoro-3-(2-(2-(methylthio)pyrimidin-4-yl)acetyl)phenyl)pivalamide (2.42 g, 6.11 mmol) in DCM (61 mL) at −5° C. (ice-brine bath) was added NBS (1.09 g, 6.11 mmol) in two portions allowing the reaction to stir for 5 min between each addition. The reaction was maintained at 5° C. for 1 h, by which time TLC analysis indicated complete conversion. The reaction was quenched with water and extracted with DCM. The organic portions were washed with brine, dried (Na₂SO₄), filtered and concentrated to afford N-(3-(2-bromo-2-(2-(methylthio)pyrimidin-4-yl)acetyl)-2-chloro-5-fluorophenyl)pivalamide (2.81 g, 5.92 mmol, 97% yield) as yellow foam which slowly crystallized upon standing: LCMS (m/z): 476.0 (MH⁺), t_{R}=1.14-1.25 min.

### Preparation of starting material N-(3-(2-bromo-2-(2-(methylthio)pyrimidin-4-yl)acetyl)-5-chloro-2-fluorophenyl)pivalamide (XII, Scheme I, (1d))

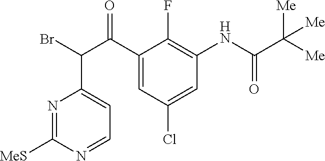 [SMILES: CSc1nccc(C(Br)C(=O)c2cc(Cl)cc(NC(=O)C(C)(C)C)c2F)n1]

### Step 1. Preparation of N-(5-chloro-2-fluoro-3-(2-(2-(methylthio)pyrimidin-4-yl)acetyl)phenyl)pivalamide

A flame-dried flask was charged with methyl methyl-5-chloro-2-fluoro-3-pivalamidobenzoate (II, 2.5 g, 8.69 mmol) and 4-methyl-2-methylthiopyridine (1.62 g, 11.30 mmol) in THF (10 mL). The solution was cooled to 0° C., and LiHMDS in THF (1.0 M, 32.1 mL, 32.1 mmol) was slowly added. The reaction mixture was stirred at 0° C. for 1 h, then at rt for 18 h. The reaction mixture was quenched with aqueous HCl solution (1.0 M, 12 mL, 12 mmol), and stirred at rt for 1 h. The mixture was partitioned between EtOAc and water, and the layers separated. The organic portion was sequentially washed with water, brine, dried (Na₂SO₄), and concentrated. The resulting residue was purified by flash chromatography on silica gel eluting with an EtOAc/hexanes (25-50%) gradient. The N-(5-chloro-2-fluoro-3-(2-(2-(methylthio)pyrimidin-4-yl)acetyl)phenyl)pivalamide (2.42 g, 6.00 mmol, 69% yield) was obtained as a yellow solid: LCMS (m/z): 396.2 (MH⁺), t_{R}=1.20 min

### Step 2. Preparation of N-(3-(2-Bromo-2-(2-(methylthio)pyrimidin-4-yl)acetyl)-5-chloro-2-fluorophenyl)pivalamide

To a stirring suspension of N-(5-chloro-2-fluoro-3-(2-(2-(methylthio)pyrimidin-4-yl)acetyl)phenyl)pivalamide (2.42 g, 6.00 mmol) in DCM (60 mL) at −5° C. was added NBS (1.07 g, 6.00 mmol) in two portions, allowing the reaction to stir for 15 min in between each addition. The reaction mixture was then stirred for 2 h at rt. The reaction was quenched with water and extracted with DCM. The combined organic extracts were washed with brine, dried (Na₂SO₄), filtered and concentrated to afford N-(3-(2-bromo-2-(2-(methylthio)pyrimidin-4-yl)acetyl)-5-chloro-2-fluorophenyl)pivalamide (2.94 g, 6.2 mmol) as a yellow foam which was carried forward without further purification:

LCMS (m/z): 476.2 (MH⁺), t_{R}=1.15 min.

### Preparation of starting material N-(3-(2-bromo-2-(2-(methylthio)pyrimidin-4-yl)acetyl)-2-fluorophenyl)propane-1-sulfonamide (XIII, Scheme III, (31))

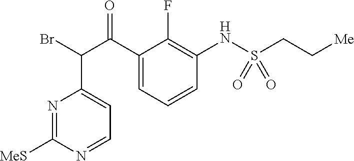 [SMILES: CCCS(=O)(=O)Nc1cccc(C(=O)C(Br)c2ccnc(SC)n2)c1F]

### Step 1. Preparation of N-(2-fluoro-3-(2-(2-(methylthio)pyrimidin-4-yl)acetyl)phenyl)propane-1-sulfonamide

This material was prepared from starting material III following the same procedure as in for starting material XI, step 1:. LCMS (m/z): 384.1 (MH⁺), t_{R}=0.97 min, broad peak; ¹H NMR (CDCl₃, 400 MHz): δ ppm 1.06 (t, 3H), 1.90 (m, 2H), 2.63 (s, 3H), 3.11 (m, 2H), 6.09 (s, 1H), 6.69 (d, J=5.48 Hz, 1H), 7.22-7.27 (m, 1H), 7.59-7.75 (m, 2H), 8.37 (d, J=5.48 Hz, 1H) from a tautomeric mixture.

### Step 2. Preparation of N-(3-(2-bromo-2-(2-(methylthio)pyrimidin-4-yl)acetyl)-2-fluorophenyl)propane-1-sulfonamide

This material was prepared from N-(2-fluoro-3-(2-(2-(methylthio)pyrimidin-4-yl)acetyl)phenyl)propane-1-sulfonamide following the same procedure as starting material XI, step 2 (96% yield): LCMS (m/z): 462.0/464.0 (MH⁺), t_{R}=0.97 min; ¹H NMR (400 MHz, CDCl₃) δ ppm 1.06 (t, J=7.43 Hz, 3H), 1.88 (m, 2H), 2.49 (s, 3H), 3.02-3.16 (m, 2H), 6.14 (s, 1H), 6.63 (br. s., 1H), 7.29 (t, 1H, J=6.7 Hz), 7.35 (d, 1H, J=5.1 Hz), 7.70 (t, J=6.7 Hz, 4H), 7.86 (t, J=7.2 Hz, 4H), 8.61 (d, J=5.09 Hz, 4H).

### Preparation of starting material 2-bromo-2-(2-chloropyrimidin-4-yl)-1-(2-fluoro-3-nitrophenyl)ethanone (XIV, Scheme IV, (4b))

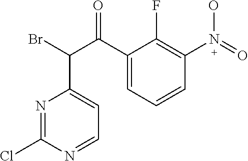

### Step 1. Preparation of 2-(2-chloropyrimidin-4-yl)-1-(2-fluoro-3-nitrophenyl)ethanone

To a solution of 2-chloro-4-methylpyrimidine (2.86 g, 22.3 mmol) and methyl 2-fluoro-3-nitrobenzoate (from intermediate III, step 1, 4.43 g, 22.3 mmol) in THF (25 mL) at 0° C., LiHMDS in THF (1.0 M, 44.5 mL, 44.5 mmol) was slowly added. The dark reaction mixture was allowed to stir at 0° C. for 2 h at which time it was quenched by the addition of 1.0 M aqueous HCl solution and allowed to stir at rt overnight. The resulting precipitate was removed by vacuum filtration. The collected filtrates were concentrated and the resulting residue was triturated with EtOH to provide 2-(2-chloropyrimidin-4-yl)-1-(2-fluoro-3-nitrophenyl)ethanone (447 mg, 1.5 mmol) as a tan solid: LCMS (m/z): 295.9 (MH⁺), t_{R}=0.98 min; ¹H NMR (400 MHz, CDCl₃) δ ppm 6.31 (s, 1H), 7.01 (d, J=5.5 Hz, 1H), 7.42 (t, J=8.6 Hz, 1H), 8.09 (m, 1H), 8.22 (m, 1H) 8.51 (d, J=5.5 Hz, 1H).

### Step 2: Preparation of 2-bromo-2-(2-chloropyrimidin-4-yl)-1-(2-fluoro-3-nitrophenyl)ethanone

To a solution of 2-(2-chloropyrimidin-4-yl)-1-(2-fluoro-3-nitrophenyl)ethanone (220 mg, 0.74 mmol) in DCM (10 ml) at −10° C., was added NBS (132 mg, 0.74 mmol) in three portions allowing the reaction to stir for 5 min in between each addition. The reaction was stirred at −10° C. for 30 min. The reaction was partitioned between DCM and water and the layers separated. The organic portion was washed with water and brine, dried (Na₂SO₄), filtered and concentrated to give 272 mg of a brown oil. The crude material was purified by flash chromatography on silica gel using an EtOAc-heptane (0-50%) elution gradient to furnish 2-bromo-2-(2-chloropyrimidin-4-yl)-1-(2-fluoro-3-nitrophenyl)ethanone (142 mg, 0.33 mmol, 44%): LCMS (m/z): 375.9 (MH⁺), t_{R}=0.86 min; ¹H NMR (400 MHz, CDCl₃) δ ppm 6.20 (s, 1H, keto form), 7.42 (td, J=8.02, 3.5 Hz, 1H enol form), 7.49 (t, J=8.0 Hz, 1H, keto form), 7.62 (d, J=6.3 Hz, 1H, enol form), 7.71 (d, J=5.1 Hz, 1H, keto form), 7.75 (m, 1H, enol form), 8.12-8.17 (m, 1H, enol form), 8.21 (m, 1H, keto form), 8.29 (m, 1H, keto form), 8.68 (d, J=6.3 Hz, 4H, enol form), 8.75 (d, J=5.5 Hz, 1H, keto form) as a tautomeric mixture.

### Preparation of starting material N-(3-(2-bromo-2-(2-chloropyrimidin-4-yl)acetyl)-2-chlorophenyl)methanesulfonamide (XV Scheme III, (3f))

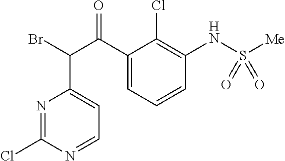 [SMILES: CS(=O)(=O)Nc1cccc(C(=O)C(Br)c2ccnc(Cl)n2)c1Cl]

### Step 1. Preparation of 1-(2-chloro-3-nitrophenyl)-2-(2-(methylthio)pyrimidin-4-yl)ethanone

This material was prepared from intermediate IV, following the same procedure as intermediate XI, step 1: LCMS (m/z): 359.9 (MH⁺), t_{R}=0.64-0.81 min; ¹H NMR (400 MHz, CDCl₃) δ ppm 3.06 (s, 3H) 5.78 (s, 1H) 6.92 (d, J=5.1 Hz, 1H) 7.34-7.47 (m, 2H) 7.77 (dd, J=7.0, 2.7 Hz, 1H) 8.47 (d, J=5.5 Hz, 1H).

### Step 2. Preparation of N-(3-(2-bromo-2-(2-chloropyrimidin-4-yl)acetyl)-2-chlorophenyl)methanesulfonamide

This material was prepared following the same procedure as intermediate XI, step 2:

LCMS (m/z): 439.9 (MH⁺), t_{R}=0.81 min; ¹H NMR (400 MHz, CDCl₃) δ ppm keto: 3.07 (s, 3H) 6.08 (s, 1H) 6.99 (s, 1H) 7.45-7.49 (m, 2H) 7.74 (d, J=5.1 Hz, 1H) 7.87 (dd, J=7.2, 2.54Hz, 1H) 8.75 (d, J=5.1 Hz, 1H); enol 3.06 (s, 3H) 6.92 (s, 1H) 7.23-7.26 (m, dl H) 7.39-7.45 (m, 1H) 7.59 (d, J=5.5 Hz, 1H) 7.77 (dd, J=8.2, 1.17 Hz, 1H) 8.66 (d, J=5.5 Hz, 1H) as a tautomeric mixture.

### Preparation of starting material N-(3-(2-bromo-2-(2-chloropyrimidin-4-yl)acetyl)-2-chlorophenyl)propane-1-sulfonamide (XVI, Scheme III, (3f))

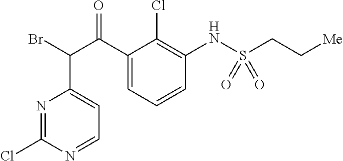 [SMILES: CCCS(=O)(=O)Nc1cccc(C(=O)C(Br)c2ccnc(Cl)n2)c1Cl]

### Step 1: Preparation of N-(2-chloro-3-(2-(2-chloropyrimidin-4-yl)acetyl)phenyl)propane-1-sulfonamide

This material was prepared from intermediate V following the same procedure as intermediate XI, step 1: LCMS (m/z): 388.0 (MH⁺), t_{R}=1.01 min; ¹H NMR (300 MHz, CDCl₃) δ ppm 1.05 (t, 3H) 1.76-1.96 (m, 2H) 2.92-3.24 (m, 2H) 4.42 (s, 1H) 5.78 (s, 1H) 6.92 (d, J=5.3 Hz, 1H) 6.95 (s, 1H) 7.34-7.38 (m, 1H) 7.39-7.42 (m, 1H) 7.74-7.82 (m, 1H) 7.86 (dd, J=6.9, 3.1 Hz, 1H) 8.47 (d, J=5.3 Hz, 1H) 8.63 (d, J=5.0 Hz, 1H).

### Step 2. N-(3-(2-bromo-2-(2-chloropyrimidin-4-yl)acetyl)-2-chlorophenyl)propane-1-sulfonamide

This material was prepared from N-(2-chloro-3-(2-(2-chloropyrimidin-4-yl)acetyl)phenyl)propane-1-sulfonamide following the same procedure as intermediate XI, step 2:LCMS (m/z): 439.9 (MH⁺), t_{R}=0.81 min; ¹H NMR (400 MHz, CDCl₃) δ ppm 0.99-1.15 (m, 6H), 1.78-1.95 (m, 4H), 3.03-3.21 (m, 4H), 6.08 (s, 1H), 6.95 (s, 1H), 7.42-7.47 (m, 2H), 7.74 (d, J=5.09 Hz, 1H), 8.75 (d, J=5.09 Hz, 1H); ¹H NMR (400 MHz, CDCl₃) δ ppm 0.98-1.11 (m, 3H), 1.77-1.97 (m, 2H), 2.99-3.28 (m, 2H), 6.89 (s, 1H), 7.15-7.24 (m, 1H), 7.35-7.42 (m, 1H), 7.59 (d, J=5.5 Hz, 1H), 7.79 (d, J=8.2 Hz, 1H), 7.89 (dd, J=6.1, 3.7 Hz, 1H), 8.66 (d, J=5.5 Hz, 1H) as a tautomeric mixture.

### Preparation of starting material N-(3-(2-bromo-2-(2-(methylthio)pyrimidin-4-yl)acetyl)-2-chlorophenyl)pivalamide (XVII, Scheme III, (3f))

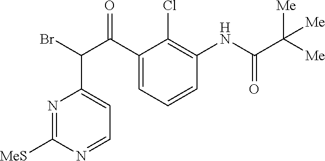 [SMILES: CSc1nccc(C(Br)C(=O)c2cccc(NC(=O)C(C)(C)C)c2Cl)n1]

### Step 1. Preparation of N-(2-chloro-3-(2-(2-(methylthio)pyrimidin-4-yl)acetyl)phenyl)pivalamide

This material was prepared from intermediate VI following the same procedure as intermediate XI, step 1: LCMS (m/z): 378.0 (MH⁺), t_{R}=1.01 min; ¹H NMR (400 MHz, CDCl₃) δ ppm 1.36 (s, 9H), 2.61 (s, 3H), 5.68 (s, 1H), 6.62 (d, J=5.1 Hz, 1H), 7.24-7.42 (m, 2H), 8.19 (br. s., 1H), 8.34 (d, J=5.5 Hz, 1H), 8.49 (dd, J=8.2, 1.6 Hz, 1H).

### Step 2. Preparation of N-(3-(2-bromo-2-(2-(methylthio)pyrimidin-4-yl)acetyl)-2-chlorophenyl)pivalamide

This material was prepared from N-(2-chloro-3-(2-(2-(methylthio)pyrimidin-4-yl)acetyl)phenyl)pivalamide following the same procedure as intermediate XI, step 2:

LCMS (m/z): 457.9 (MH⁺), t_{R}=1.02-1.12 min; ¹H NMR (400 MHz, CDCl₃) δ ppm 1.37 (s, 18H), 2.54 (s, 3H), 2.64 (s, 3H), 6.06 (s, 1H), 7.12 (d, J=7.4Hz, 1H), 7.24-7.44 (m, 4H), 8.14 (br. s., 2H), 8.48-8.56 (m, 2H), 8.57-8.65 (m, 1H) as a tautomeric mixture.

### Preparation of starting material N-(3-(2-bromo-2-(2-(methylthio)pyrimidin-4-yl)acetyl)-2,5-dichlorophenyl)pivalamide (XVIII, Scheme III, (31))

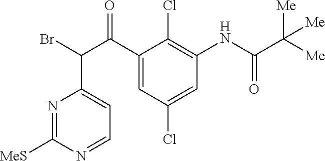 [SMILES: CSc1nccc(C(Br)C(=O)c2cc(Cl)cc(NC(=O)C(C)(C)C)c2Cl)n1]

### Step 1. Preparation of N-(2,5-dichloro-3-(2-(2-(methylthio)pyrimidin-4-yl)acetyl)phenyl)pivalamide

This material was prepared from intermediate VII following the same procedure as starting material XI, step 1: LCMS (m/z): 412 (MH⁺), t_{R}=1.23 min.

Step 2. Preparation of N-(3-(2-bromo-2-(2-(methylthio)pyrimidin-4-yl)acetyl)-2,5-dichlorophenyl)pivalamide:

This material was prepared from N-(2,5-dichloro-3-(2-(2-(methylthio)pyrimidin-4-yl)acetyl)phenyl)pivalamide following the same procedure as starting material XI, step 2:

LCMS (m/z): 492 (MH⁺), t_{R}=1.16-1.28 min.

### Preparation of starting material N-(3-(2-bromo-2-(2-(methylthio)pyrimidin-4-yl)acetyl)-2,5-dichlorophenyl)propane-1-sulfonamide (XIX, Scheme III, (31))

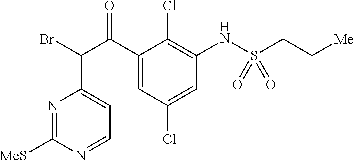 [SMILES: CCCS(=O)(=O)Nc1cc(Cl)cc(C(=O)C(Br)c2ccnc(SC)n2)c1Cl]

Step 1. Preparation of N-(2,5-dichloro-3-(2-(2-(methylthio)pyrimidin-4-yl)acetyl)phenyl)propane-1-sulfonamide:

This material was prepared from intermediate IX following the same procedure as intermediate XI, step 1: LCMS (m/z): 434 (MH⁺), t_{R}=1.14 min.

### Step 2. Preparation of N-(3-(2-bromo-2-(2-(methylthio)pyrimidin-4-yl)acetyl)-2,5-dichlorophenyl)propane-1-sulfonamide

This material was prepared from N-(2,5-dichloro-3-(2-(2-(methylthio)pyrimidin-4-yl)acetyl)phenyl)propane-1-sulfonamide following the same procedure as starting material X/, step 2 LCMS (m/z): 514 (MH⁺), t_{R}=1.09-1.20 min.

### Preparation of starting material N-(3-(2-bromo-2-(2-(methylthio)pyrimidin-4-yl)acetyl)-2,5-dichlorophenyl)methanesulfonamide (XX, Scheme III, (3f))

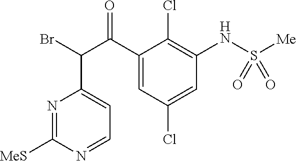 [SMILES: CSc1nccc(C(Br)C(=O)c2cc(Cl)cc(NS(C)(=O)=O)c2Cl)n1]

### Step 1. Preparation of N-(2,5-dichloro-3-(2-(2-(methylthio)pyrimidin-4-yl)acetyl)phenyl)methanesulfonamide

This material was prepared from intermediate VIII following the same procedure as intermediate XI, step 1: LCMS (m/z): 406 (MH⁺), t_{R}=1.03 min.

### Step 2. Preparation of N-(3-(2-bromo-2-(2-(methylthio)pyrimidin-4-yl)acetyl)-2,5-dichlorophenyl)methanesulfonamide

This material was prepared from N-(2,5-dichloro-3-(2-(2-(methylthio)pyrimidin-4-yl)acetyl)phenyl)methanesulfonamide following the same procedure as intermediate XI, step 2

LCMS (m/z): 486 (MH⁺), t_{R}=0.98-1.09 min.

### Preparation of starting material 3-(2-tert-Butyl-5-(2-(methylthio)pyrimidin-4-yl)thiazol-4-yl)-2-chloro-5-fluoroaniline (XXI, Scheme I, (1f))

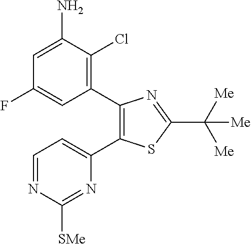 [SMILES: CSc1nccc(-c2sc(C(C)(C)C)nc2-c2cc(F)cc(N)c2Cl)n1]

### Step 1. Preparation of N-(3-(2-tert-butyl-5-(2-(methylthio)pyrimidin-4-yl)thiazol-4-yl)-2-chloro-5-fluorophenyl)pivalamide

To a solution of N-(3-(2-bromo-2-(2-(methylthio)pyrimidin-4-yl)acetyl)-2-chloro-5-fluorophenyl)-pivalamide (XL 489 mg, 1.03 mmol) in DMA (4.7 mL) under Ar at rt was added 2,2-dimethylpropanethioamide (131 mg, 1.12 mmol). The reaction mixture was stirred at rt for 1 h, and then heated to and maintained at 80° C. for 3 h. The reaction was judged complete by LCMS. The reaction mixture was diluted with water and twice extracted with EtOAc. The organic phases were combined, washed with brine, dried (Na₂SO₄), and concentrated. The resulting residue was adsorbed onto silica gel and purified by flash chromatography on silica gel using an EtOAc:heptanes (0-15%) elution gradient to furnish N-(3-(2-tert-butyl-5-(2-(methylthio)pyrimidin-4-yl)thiazol-4-yl)-2-chloro-5-fluorophenyl)pivalamide (175 mg, 0.36 mmol, 38%) as a white foam: LCMS (m/z): 493.1 (MH⁺), t_{R}=1.42 min.

### Step 2. Preparation of 3-(2-tert-butyl-5-(2-(methylthio)pyrimidin-4-yl)thiazol-4-yl)-2-chloro-5-fluoroaniline

A solution of N-(3-(2-tert-butyl-5-(2-(methylthio)pyrimidin-4-yl)thiazol-4-yl)-2-chloro-5-fluorophenyl)pivalamide (175 mg, 0.355 mmol) in EtOH (3.6 mL) was treated with 6.0 M aqueous HCl solution (1.8 mL, 10.7 mmol). The reaction mixture was heated in oil bath at 80° C. for 18 h, then cooled to rt, diluted with EtOAc and quenched with aqueous saturated NaHCO₃ to pH 8. The phases were separated and the aqueous layer was twice extracted with EtOAc. The organic portions were combined, washed with water, brine, dried (Na₂SO₄), filtered and concentrated. The resulting residue was adsorbed onto silica gel and purified by flash chromatography on silica gel using an EtOAc:heptane (0-50%) elution gradient to afford 3-(2-tert-butyl-5-(2-(methylthio)pyrimidin-4-yl)thiazol-4-yl)-2-chloro-5-fluoroaniline (104 mg, 0.25 mmol, 71% yield) as a white foam: LCMS (m/z): 409.1 (MH⁺), t_{R}=1.17 min.

### Preparation of starting material 3-(2-tert-butyl-5-(2-(methylthio)pyrimidin-4-yl)thiazol-4-yl)-5-chloro-2-fluoroaniline (XXII, Scheme I, (1f))

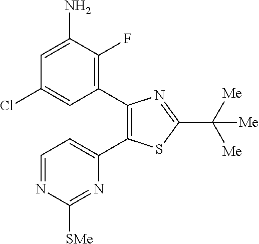 [SMILES: CSc1nccc(-c2sc(C(C)(C)C)nc2-c2cc(Cl)cc(N)c2F)n1]

### Step 1: Preparation of N-(3-(2-tert-Butyl-5-(2-(methylthio)pyrimidin-4-yl)thiazol-4-yl)-5-chloro-2-fluorophenyl)-pivalamide

This material was prepared from intermediate XII following the procedure for intermediate XXI, step 1: LCMS (m/z): 493.0 (MH⁺), t_{R}=1.39 min; ¹H NMR (400 MHz, CDCl₃) δ ppm 1.35 (s, 9H) 1.38 (s, 9H) 2.57-2.66 (m, 3H) 7.52-7.59 (m, 1H) 7.66-7.74 (m, 1H) 8.33-8.40 (m, 1H) 8.48-8.57 (m, 1H).

Step 2. Preparation of 3-(2-tert-butyl-5-(2-(methylthio)pyrimidin-4-yl)thiazol-4-yl)-5-chloro-2-fluoroaniline:

The titled compound was prepared from N-(3-(2-tert-Butyl-5-(2-(methylthio)pyrimidin-4-yl)thiazol-4-yl)-5-chloro-2-fluorophenyl)-pivalamide following the procedure for intermediate XX, step 2: LCMS (m/z): 409.0 (MH⁺), t_{R}=1.26 min.

### Preparation of starting material N-(3-(2-tert-butyl-5-(2-chloropyrimidin-4-yl)thiazol-4-yl)-2-fluorophenyl)methanesulfonamide POOH, Scheme IV, (1g)

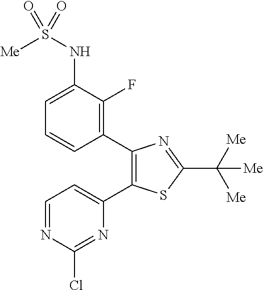 [SMILES: CC(C)(C)c1nc(-c2cccc(NS(C)(=O)=O)c2F)c(-c2ccnc(Cl)n2)s1]

### Step 1. 2-tert-butyl-5-(2-chloropyrimidin-4-yl)-4-(2-fluoro-3-nitrophenyl)thiazole

The desired material was prepared from starting material XIV following the procedure used for starting material XXI, step 1: LCMS (m/z): 393.0 (MH⁺), t_{R}=1.13 min;

¹H NMR (400 MHz, CDCl₃) δ ppm 1.51 (s, 9H), 7.01 (d, J=5.1 Hz, 1H), 7.33-7.52 (m, 1H), 7.78-7.97 (m, 1H), 8.03-8.24 (m, 1H), 8.48 (d, J=5.1 Hz, 1H).

### Step 2. Preparation of 3-(2-tert-butyl-5-(2-chloropyrimidin-4-yl)thiazol-4-yl)-2-fluoroaniline

2-tert-butyl-5-(2-chloropyrimidin-4-yl)-4-(2-fluoro-3-nitrophenyl)thiazole (34 mg, 0.087 mmol) was dissolved in HOAc (3 mL) and iron (48 mg, 0.87 mmol) was added. The reaction mixture was stirred at rt for 24 h. LCMS showed that small amount of starting material was still present. The reaction mixture was diluted with EtOAc, filtered and the filtrate evaporated. The residue was partitioned between saturated aqueous NaHCO₃ solution and EtOAc and the layers were separated. The water phase was extracted with EtOAc. The combined organic extracts were washed with water, brine and dried (Na₂SO₄). The crude material was adsorbed on silica gel and purified by flash chromatography on silica gel (2:1 to 1:1 heptane-EtOAc) to give the titled intermediate (5.9 mg, 0.016 mmol):

LCMS (m/z): 363.0 (MH⁺), t_{R}=1.03 min.

### Step 3. Preparation of N-(3-(2-tert-butyl-5-(2-chloropyrimidin-4-yl)thiazol-4-yl)-2-fluorophenyl)methanesulfonamide

The titled intermediated was prepared from 3-(2-tert-butyl-5-(2-chloropyrimidin-4-yl)thiazol-4-yl)-2-fluoroaniline following the sulfonylation procedure used for the preparation of starting material IV, step 3, using pyridine as the solvent: LCMS (m/z): 441.1 (MH⁺), t_{R}=1.04 min.

### Preparation of starting material N-(3-(2-tert-butyl-5-(2-chloropyrimidin-4-yl)thiazol-4-yl)-2-fluorophenyl)propanesulfonamide (XXIV, Scheme IV, (1g)

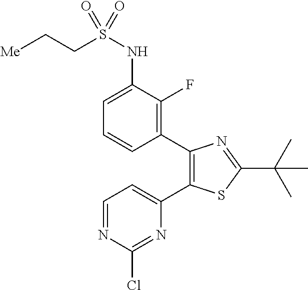 [SMILES: CCCS(=O)(=O)Nc1cccc(-c2nc(C(C)(C)C)sc2-c2ccnc(Cl)n2)c1F]

The desired compound was obtained by sulfonylating 3-(2-tert-butyl-5-(2-chloropyrimidin-4-yl)thiazol-4-yl)-2-fluoroaniline (intermediate XXIII, step 2) with 1-propanesulfonyl chloride according to the procedure for intermediate IV, step 3, using pyridine as the solvent.

LCMS (m/z): 469.0 (MH⁺), t_{R}=1.11 min.

### Preparation of starting material N-(3-(2-tert-butyl-5-(2-chloropyrimidin-4-yl)thiazol-4-yl)-2-chlorophenyl)methanesulfonamide (XXV, Scheme III, (1g))

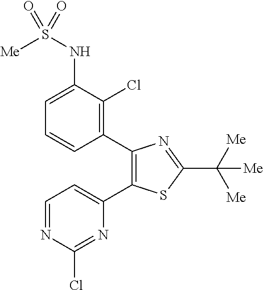 [SMILES: CC(C)(C)c1nc(-c2cccc(NS(C)(=O)=O)c2Cl)c(-c2ccnc(Cl)n2)s1]

The desired compound was obtained from intermediate XV following the procedure used for intermediate XX/, step 1: ¹H NMR (400 MHz, CDCl₃) δ ppm 1.51 (s, 9H) 3.08 (s, 3H) 6.66 (d, J=5.5 Hz, 1H) 6.94 (s, 1H) 7.28 (dd, J=7.8, 1.6 Hz, 1H) 7.45 (t, J=8.0 Hz, 1H) 7.82 (dd, J=8.2, 1.17 Hz, 1H) 8.34 (d, J=5.5 Hz, 1H).

### Preparation of starting material N-(3-(2-tert-butyl-5-(2-chloropyrimidin-4-yl)thiazol-4-yl)-2-chlorophenyl)propanesulfonamide (XXVI, Scheme III, (1g))

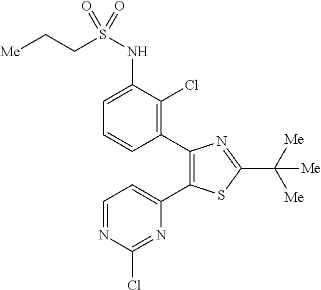 [SMILES: CCCS(=O)(=O)Nc1cccc(-c2nc(C(C)(C)C)sc2-c2ccnc(Cl)n2)c1Cl]

The desired compound was obtained from intermediate XVI following the procedure used for starting material XX/, step 1: LCMS (m/z): 485.2 (MH⁺), t_{R}=1.2 min;

¹H NMR (400 MHz, CDCl₃) δ ppm 1.06 (t, J=7.4Hz, 3H), 1.51 (s, 9H), 1.89 (m, 2H), 3.09-3.18 (m, 2H), 6.64 (d, J=5.5 Hz, 1H), 6.92 (s, 1H), 7.19-7.30 (m, 1H), 7.43 (t, J=7.8 Hz, 1H) 7.83 (dd, J=8.2, 1.2 Hz, 1H), 8.33 (d, J=5.1 Hz, 1H).

### Preparation of N-(3-(2-tert-butyl-5-(2-(methylthio)pyrimidin-4-yl)thiazol-4-yl)-2,5-dichlorophenyl)propane-1-sulfonamide (XXVII, Scheme III, (1g))

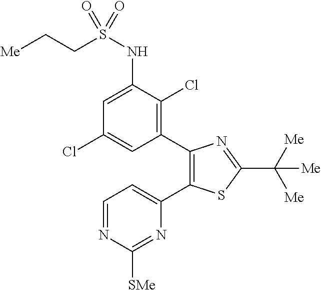 [SMILES: CCCS(=O)(=O)Nc1cc(Cl)cc(-c2nc(C(C)(C)C)sc2-c2ccnc(SC)n2)c1Cl]

The titled compound was obtained from intermediate XIX following the procedure used for intermediate XXI.

LCMS (m/z): 531 (MH⁺), t_{R}=1.30 min.

### Preparation of N-(3-(2-tert-butyl-5-(2-(methylthio)pyrimidin-4-yl)thiazol-4-yl)-2,5-dichlorophenyl)methanesulfonamide (XXVIII, Scheme III, (1g))

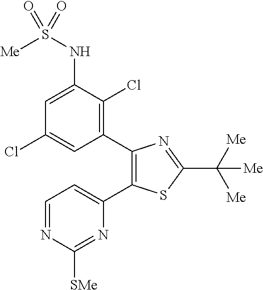 [SMILES: CSc1nccc(-c2sc(C(C)(C)C)nc2-c2cc(Cl)cc(NS(C)(=O)=O)c2Cl)n1]

The titled compound was obtained from intermediate XX following the procedure used for starting material XXI.

LCMS (m/z): 503/505 (MH⁺), t_{R}=1.22 min.

### Preparation of starting material 2-chloro-3-(2-cyclopropyl-5-(2-(methylthio)pyrimidin-4-yl)thiazol-4-yl)-5-fluoroaniline (XXIX, Scheme V, (10

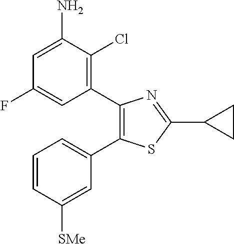 [SMILES: CSc1cccc(-c2sc(C3CC3)nc2-c2cc(F)cc(N)c2Cl)c1]

### Step 1. Preparation of N-(3-(2-amino-5-(2-(methylthio)pyrimidin-4-yl)thiazol-4-yl)-2-chloro-5-fluorophenyl)pivalamide

To a solution of N-(3-(2-bromo-2-(2-(methylthio)pyrimidin-4-yl)acetyl)-2-chloro-5-fluorophenyl)pivalamide (intermediate XI, 489 mg, 1.03 mmol) in EtOH (5.1 mL) at rt was added thiourea (392 mg, 5.15 mmol). The reaction mixture was allowed to stir for 15 min at rt, then heated to, and maintained at, 75° C. for 30 min in an oil bath. LCMS indicated complete conversion and the reaction was allowed to cool to rt. The reaction was diluted with water and twice extracted with EtOAc. The combined organic phases were washed with brine, dried (Na₂SO₄), and concentrated. The resulting residue was adsorbed onto silica gel and purified by flash chromatography on silica gel using an heptane-EtOAc (20-100%) elution gradient to furnish N-(3-(2-amino-5-(2-(methylthio)pyrimidin-4-yl)thiazol-4-yl)-2-chloro-5-fluorophenyl)pivalamide (311 mg, 0.68 mmol, 66% yield) as a pale yellow solid: LCMS (m/z): 452.2 (MH⁺), t_{R}=0.88 min.

### Step 2. Preparation of N-(3-(2-bromo-5-(2-(methylthio)pyrimidin-4-yl)thiazol-4-yl)-2-chloro-5-fluorophenyl)pivalamide

To an oven dried round bottom flask under nitrogen containing a solution of copper(II) bromide (154 mg, 0.69 mmol) in ACN (7 mL) at 0° C. (ice-brine bath) was added tert-butylnitrite (123 μL, 1.03 mmol) over 10 min. To this cold reaction mixture was added a suspension of N-(3-(2-amino-5-(2-(methylthio)-pyrimidin-4-yl)thiazol-4-yl)-2-chloro-5-fluorophenyl)pivalamide (311 mg, 0.69 mmol) in ACN (6.9 mL) over 10 min. The reaction mixture was then heated to 65° C. for 2 h. LCMS of an aliquot indicated complete conversion. The reaction mixture was concentrated and then diluted with EtOAc and water. The phases were partitioned and the aqueous phase was extracted with EtOAc. The organic phases were combined, washed with water, brine, dried (Na₂SO₄), filtered and concentrated to afford N-(3-(2-bromo-5-(2-(methylthio)pyrimidin-4-yl)thiazol-4-yl)-2-chloro-5-fluorophenyl)pivalamide (320 mg, 0.59 mmol, 86% yield) as a brown foam: LCMS (m/z): 517.1 (MH⁺), t_{R}=1.29 min.

### Step 3. Preparation of N-(2-chloro-3-(2-cyclopropyl-5-(2-(methylthio)pyrimidin-4-yl)thiazol-4-yl)-5-fluorophenyl)pivalamide

A mixture of N-(3-(2-bromo-5-(2-(methylthio)pyrimidin-4-yl)thiazol-4-yl)-2-chloro-5-fluorophenyl)pivalamide (320 mg, 0.620 mmol), cyclopropylboronic acid pinacol ester (417 mg, 2.48 mmol), potassium phosphate (395 mg, 1.86 mmol) in toluene (5.2 mL) and water (1.0 mL) was sparged with nitrogen. Pd(OAc)₂ (28 mg, 0.124 mmol) and tricyclohexylphosphine (70 mg, 0.25 mmol) was then added and the reaction mixture was sealed in a reaction tube and heated in an oil bath to 100° C. for 18 h. LCMS indicated 10-20% conversion. Additional cyclopropylboronic acid (213 mg, 2.48 mmol) was introduced and the reaction was maintained for another 18 h at 100° C. LCMS of an aliquot indicated 90% conversion. The reaction mixture was allowed to cool to rt, diluted with water and twice extracted with EtOAc. The organic phases were combined, washed with brine, dried (Na₂SO₄), filtered and concentrated. The resulting residue was adsorbed onto silica gel and purified by flash chromatography on silica gel using a heptane-EtOAc (1-50%) elution gradient to give N-(2-chloro-3-(2-cyclopropyl-5-(2-(methylthio)pyrimidin-4-yl)thiazol-4-yl)-5-fluorophenyl)pivalamide (107 mg, 0.22 mmol, 36% yield) as a light brown foam: LCMS (m/z): 477.2 (MH⁺), t_{R}=1.24 min.

### Step 4. Preparation of 2-chloro-3-(2-cyclopropyl-5-(2-(methylthio)pyrimidin-4-yl)thiazol-4-yl)-5-fluoroaniline

A solution of N-(2-chloro-3-(2-cyclopropyl-5-(2-(methylthio)pyrimidin-4-yl)thiazol-4-yl)-5-fluorophenyl)pivalamide (107 mg, 0.224 mmol) in ethanol (2.2 mL) was treated with an aqueous sulfuric acid solution (50% v/v, 1.2 mL). The resulting reaction mixture was heated in oil bath at 90° C. for 8 h. LCMS of an aliquot indicated 95% conversion. The reaction mixture was allowed to cool to rt and was then carefully added to a biphasic solution of EtOAc and saturated aqueous NaHCO₃ solution (basified to pH 7). The phases were separated and aqueous layer was extracted with EtOAc. The organic portions were combined, washed with brine, dried (Na₂SO₄), and concentrated to afford 2-chloro-3-(2-cyclopropyl-5-(2-(methylthio)pyrimidin-4-yl)thiazol-4-yl)-5-fluoroaniline (86 mg, 0.22 mmol, 98% yield) as a orange foam: LCMS (m/z): 393.1 (MH⁺), t_{R}=1.08 min

### Preparation of 5-chloro-3-(2-cyclopropyl-5-(2-(methylthio)pyrimidin-4-yl)thiazol-4-yl)-2-fluoroaniline (XXX, Scheme V, (1f)

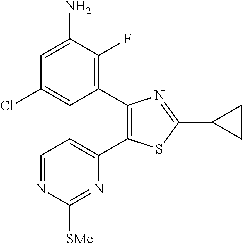 [SMILES: CSc1nccc(-c2sc(C3CC3)nc2-c2cc(Cl)cc(N)c2F)n1]

### Step 1. N-(3-(2-Amino-5-(2-(methylthio)pyrimidin-4-yl)thiazol-4-yl)-5-chloro-2-fluorophenyl)pivalamide

A mixture of N-(3-(2-bromo-2-(2-(methylthio)pyrimidin-4-yl)acetyl)-5-chloro-2-fluorophenyl)pivalamide (XII, 467 mg, 0.98 mmol) and thiourea (374 mg, 4.91 mmol) in EtOH was heated at 90° C. for 45 min and then allowed to cool to rt. The reaction mixture was diluted with EtOAc (50 ml), washed with saturated aqueous NaHCO₃ solution (25 ml), then brine (25 ml), dried (Na₂SO₄), and concentrated. The resulting residue was purified by flash chromatography on silica gel eluting with an EtOAc-heptane (0-50%) elution gradient and the desired product (484 mg) was obtained as a yellow solid: LCMS (m/z): 452.2 (MH⁺), t_{R}=0.89 min.

### Step 2. N-(3-(2-Bromo-5-(2-(methylthio)pyrimidin-4-yl)thiazol-4-yl)-5-chloro-2-fluorophenyl)pivalamide

To a solution of copper(II) bromide (201 mg, 0.90 mmol) in ACN (10 mL) at 0° C., t-butyl nitrite (139 mg, 1.35 mmol) was added. To this reaction mixture a slurry of N-(3-(2-amino-5-(2-(methylthio)pyrimidin-4-yl)thiazol-4-yl)-5-chloro-2-fluorophenyl)pivalamide (480 mg, 0.90 mmol) in ACN (15 mL) was added over 10 min. The reaction was then heated to 65° C. for 2 h and LCMS indicated only partial conversion. Additional ACN (25 ml) and t-butyl nitrite (139 mg, 1.35 mmol) were sequentially added and the reaction mixture was stirred at 65° C. for an additional 2.5 h. The reaction mixture was allowed to cooled to rt, concentrated and the resulting residue was purified by flash chromatography on silica gel eluting with EtOAc-heptane (0-30%). The desired product (405 mg, 87% yield) was obtained as a light yellow solid: LCMS (m/z): 517.0 (MH⁺), t_{R}=1.28 min.

### Step 3. N-(5-chloro-3-(2-cyclopropyl-5-(2-(methylthio)pyrimidin-4-yl)thiazol-4-yl)-2-fluorophenyl)pivalamide

A mixture of N-(3-(2-bromo-5-(2-(methylthio)pyrimidin-4-yl)thiazol-4-yl)-5-chloro-2-fluorophenyl)pivalamide (400 mg, 0.78 mmol), cyclopropylboronic acid (200 mg, 2.33 mmol) and potassium phosphate (988 mg, 4.65 mmol) in toluene (5 mL) and water (0.50 mL) was purged with Ar. Pd(OAc)₂ (34.8 mg, 0.155 mmol) and tricyclohexylphosphine (87 mg, 0.31 mmol) were added, followed by an Ar purge. The reaction was then heated at 100° C. overnight. The reaction mixture was allowed to cool to rt, partitioned between EtOAc and water, and the layers separated. The organic portion was washed with water, brine, dried, and concentrated. The resulting residue was purified by flash chromatography on silica gel eluting with heptane-EtOAc (0-30%) and the desired product (143 mg, 39% yield) was obtained as an off white solid: LCMS (m/z): 477.1 (MH⁺), t_{R}=1.24 min.

### Step 4. Preparation of 5-chloro-3-(2-cyclopropyl-5-(2-(methylthio)pyrimidin-4-yl)thiazol-4-yl)-2-fluoroaniline

A solution of N-(5-chloro-3-(2-cyclopropyl-5-(2-(methylthio)pyrimidin-4-yl)thiazol-4-yl)-2-fluorophenyl)pivalamide (143 mg, 0.300 mmol) in EtOH (9 ml) was treated with aqueous H₂SO₄ (50% v/v, 3 ml) and the reaction mixture was heated at reflux for 8 h. The reaction mixture was allowed to cool to rt, concentrated, and the resulting residue was dissolved in ice water (30 ml), neutralized by solid NaHCO₃ (6 g) and extracted with EtOAc (2×30 ml). The organic extracts were combined, washed with brine (30 ml), dried (Na₂SO₄), concentrated and crude 5-chloro-3-(2-cyclopropyl-5-(2-(methylthio)pyrimidin-4-yl)thiazol-4-yl)-2-fluoroaniline was obtained as a yellow residue which was used without further purification: LCMS (m/z): 393.2 (MH⁺), t_{R}=1.10 min; ¹H NMR (400 MHz, CDCl₃) δ ppm 1.10-1.20 (m, 2H), 1.20-1.24 (m, 2H), 2.30-2.43 (m, 1H), 2.52 (s, 3H), 3.90 (s, 2H) 6.71 (d, J=5.1 Hz, 1H) 6.79-6.89 (m, 2H) 8.31 (d, J=5.5 Hz, 1H).

### Preparation of starting material N-(3-(2-cyclopropyl-5-(2-(methylsulfonyl)pyrimidin-4-yl)thiazol-4-yl)-2-fluorophenyl)propane-1-sulfonamide (XXXI, Scheme V, (1h)

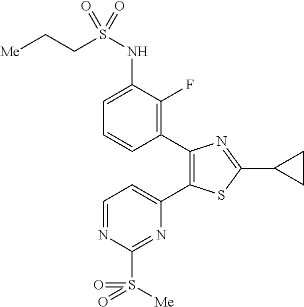 [SMILES: CCCS(=O)(=O)Nc1cccc(-c2nc(C3CC3)sc2-c2ccnc(S(C)(=O)=O)n2)c1F]

### Step 1. N-(3-(2-amino-5-(2-(methylthio)pyrimidin-4-yl)thiazol-4-yl)-2-fluorophenyl)propane-1-sulfonamide

The titled intermediate was obtained from intermediate XIII following the procedure for intermediate XXIX, step 1: LCMS (m/z): 440.1 (MH⁺), 481.1 (M+ACN), t_{R}=0.72 min; ¹H NMR (400 MHz, CDCl₃) δ ppm 0.99 (t, J=7.6 Hz, 3H), 1.86 (m, 2H), 2.46 (s, 3H), 3.02 (m, 2H), 5.68 (s, 2H), 6.38 (d, J=5.1 Hz, 1H), 7.13 (t, J=7.8 Hz, 1H) 7.22-7.29 (m, 1H, partially covered by solvent signal), 7.42 (t, J=7.8 Hz, 1H), 8.15 (d, J=5.5 Hz, 1H) 8.51 (br. s., 1H).

### Step 2. N-(3-(2-bromo-5-(2-(methylthio)pyrimidin-4-yl)thiazol-4-yl)-2-fluorophenyl)propane-1-sulfonamide

The titled intermediate was obtained from intermediate XIII following the procedure for intermediate XXIX, step 2: LCMS (m/z): 503.1 (MH⁺), t_{R}=1.11 min.

### Step 3. N-(3-(2-cyclopropyl-5-(2-(methylthio)pyrimidin-4-yl)thiazol-4-yl)-2-fluorophenyl)propane-1-sulfonamide

The titled intermediate was obtained from intermediate XIII following the procedure for intermediate XXIX, step 3: LCMS (m/z): 465.2 (MH⁺), t_{R}=1.05 min.

### Step 4. N-(3-(2-cyclopropyl-5-(2-(methylsulfonyl)pyrimidin-4-yl)thiazol-4-yl)-2-fluorophenyl)propane-1-sulfonamide

To a solution of N-(3-(2-cyclopropyl-5-(2-(methylthio)pyrimidin-4-yl)thiazol-4-yl)-2-fluorophenyl)propane-1-sulfonamide (306 mg, 0.66 mmol) in DCM (15 ml) at rt was added 70% mCPBA (325 mg, 1.32 mmol) and the reaction was allowed to stir at rt overnight. The reaction was quenched with aqueous saturated sodium bicarbonate, the phases were separated. The water phase was acidified to pH 5 and extracted with DCM. The combined organic portions were dried (Na₂SO₄), and concentrated to provide N-(3-(2-cyclopropyl-5-(2-(methylsulfonyl)pyrimidin-4-yl)thiazol-4-yl)-2-fluorophenyl)propane-1-sulfonamide (298 mg, 0.600 mmol) which was used without further purification: LCMS (m/z): 497.2. (MH⁺), t_{R}=0.85 min.

### Preparation of starting material N-(2-chloro-3-(2-cyclopropyl-5-(2-(methylsulfonyl)pyrimidin-4-yl)thiazol-4-yl)phenyl)propane-1-sulfonamide (XXXII, Scheme V, (1h)

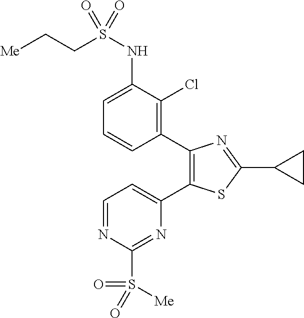 [SMILES: CCCS(=O)(=O)Nc1cccc(-c2nc(C3CC3)sc2-c2ccnc(S(C)(=O)=O)n2)c1Cl]

### Step 1. Preparation of N-(3-(2-amino-5-(2-(methylthio)pyrimidin-4-yl)thiazol-4-yl)-2-chlorophenyl)pivalamide

The titled intermediate was obtained from intermediate XVII following the procedure for intermediate XXIX, step 1: LCMS (m/z): 434.2 (MH⁺), t_{R}=0.78 min; ¹H NMR (400 MHz, CDCl₃) δ ppm 1.35 (s, 9H), 2.51 (s, 3H), 5.28 (s, 2H), 6.28 (d, J=5.5 Hz, 1H), 7.12-7.16 (m, 1H), 7.39 (t, J=8.0 Hz, 1H) 8.14 (d, J=5.48 Hz, 2H) 8.58 (dd, J=8.2, 1.6 Hz, 1H).

### Step 2. Preparation of N-(3-(2-bromo-5-(2-(methylthio)pyrimidin-4-yl)thiazol-4-yl)-2-chlorophenyl)pivalamide

The titled intermediate was obtained from material above following the procedure for intermediate XXIX, step 2: LCMS (m/z): 499.1 (MH⁺), t_{R}=1.23 min; ¹H NMR (400 MHz, CDCl₃) δ ppm 1.36 (s, 9H), 2.55 (s, 3H), 6.42 (d, J=5.1 Hz, 1H), 7.15 (dd, J=7.4, 1.6 Hz, 1H), 7.41 (t, J=8.0 Hz, 1H), 8.12 (s, 1H), 8.30 (d, J=5.5 Hz, 1H), 8.61 (dd, J=8.6, 1.6 Hz, 1H).

### Step 3. Preparation of N-(2-chloro-3-(2-cyclopropyl-5-(2-(methylthio)pyrimidin-4-yl)thiazol-4-yl)phenyl)pivalamide

The titled intermediate was obtained from material above following the procedure for intermediate XXIX, step 3: LCMS (m/z): 459.1 (MH⁺), t_{R}=1.10 min; ¹H NMR (400 MHz, CDCl₃) δ ppm 1.13-1.31 (m, 4H), 1.35 (s, 9H), 2.30-2.45 (m, 1H), 2.52 (s, 3H) 6.41 (d, J=5.1 Hz, 1H), 7.13 (d, J=7.4Hz, 1H), 7.39 (t, J=8.0 Hz, 1H), 8.14 (s, 1H) 8.23 (d, J=5.5 Hz, 1H), 8.57 (d, J=8.2 Hz, 1H).

### Step 4. Preparation of 2-chloro-3-(2-cyclopropyl-5-(2-(methylthio)pyrimidin-4-yl)thiazol-4-yl)aniline

The titled intermediate was obtained from material above following the procedure for intermediate XXIX, step 4: LCMS (m/z): 375.1 (MH⁺), t_{R}=0.61 min; ¹H NMR (400 MHz, CDCl₃) δ ppm 1.13-1.30 (m, 4H), 2.38-2.49 (m, 1H), 2.55 (s, 3H), 2.62-2.93 (m, 2H), 6.48 (d, J=5.5 Hz, 1H), 6.74-6.83 (m, 1H), 6.90 (d, J=6.7 Hz, 1H), 7.18 (t, J=7.8 Hz, 1H), 8.24 (d, J=5.5 Hz, 1H).

### Step 5. Preparation of N-(2-chloro-3-(2-cyclopropyl-5-(2-(methylthio)pyrimidin-4-yl)thiazol-4-yl)phenyl)propane-1-sulfonamide

The titled intermediate was prepared from 2-chloro-3-(2-cyclopropyl-5-(2-(methylthio)pyrimidin-4-yl)thiazol-4-yl)aniline in a manner similar to intermediate IV, step 4 using 1-propanesulfonyl chloride: LCMS (m/z): 481.2 (MH⁺), t_{R}=1.07 min.

### Step 6. Preparation of N-(2-chloro-3-(2-cyclopropyl-5-(2-(methylsulfonyl)pyrimidin-4-yl)thiazol-4-yl)phenyl)propane-1-sulfonamide

The titled intermediate was prepared from N-(2-chloro-3-(2-cyclopropyl-5-(2-(methylthio)pyrimidin-4-yl)thiazol-4-yl)phenyl)propane-1-sulfonamide in a manner similar to intermediate XXXI, step 4: LCMS (m/z): 513.2 (MH⁺), t_{R}=0.87 min.

### Preparation of starting material N-(2-chloro-3-(2-cyclopropyl-5-(2-(methylsulfonyl)pyrimidin-4-yl)thiazol-4-yl)phenyl)methanesulfonamide (XXXIII, Scheme V, (1h)

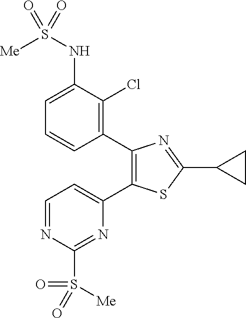 [SMILES: CS(=O)(=O)Nc1cccc(-c2nc(C3CC3)sc2-c2ccnc(S(C)(=O)=O)n2)c1Cl]

### Step 1. N-(2-chloro-3-(2-cyclopropyl-5-(2-(methylthio)pyrimidin-4-yl)thiazol-4-yl)phenyl)methanesulfonamide

The titled intermediate was prepared from 2-chloro-3-(2-cyclopropyl-5-(2-(methylthio)pyrimidin-4-yl)thiazol-4-yl)aniline (intermediate XXXI, step 4) in a manner similar to intermediate IV, step 4 using methanesulfonyl chloride: LCMS (m/z): 403.1 (MH⁺), t_{R}=1.19 min; ¹H NMR (400 MHz, CDCl₃) δ ppm 1.14-1.24 (m, 7H), 1.33 (t, J=7.2 Hz, 4H), 2.32-2.42 (m, 1H), 2.54 (s, 3H), 3.19-3.32 (m, 2H), 4.37 (t, J=4.9 Hz, 1H), 6.45 (d, J=5.5 Hz, 1H), 6.69 (dd, J=7.6, 1.4Hz, 1H), 6.75 (dd, J=8.2, 1.2 Hz, 1H), 7.23 (t, J=7.6 Hz, 1H) 8.19 (d, J=5.5 Hz, 1H).

### Step 2. N-(2-chloro-3-(2-cyclopropyl-5-(2-(methylsulfonyl)pyrimidin-4-yl)thiazol-4-yl)phenyl)methanesulfonamide

The titled intermediate was obtained from the material above following the procedure for intermediate XXXI, step 4: LCMS (m/z): 485.2 (MH⁺), t_{R}=0.74 min; ¹H NMR (400 MHz, CDCl₃) δ ppm 1.14-1.35 (m, 4H) 2.32-2.47 (m, 1H) 3.10 (s, 3H) 3.25 (s, 3H) 6.94 (d, J=5.48 Hz, 1H) 6.97 (s, 1H) 7.22-7.30 (m, 1H) 7.46 (t, J=8.02 Hz, 1H) 7.83 (dd, J=8.22, 1.17 Hz, 1H) 8.63 (d, J=5.48 Hz, 1H).

### Preparation of N-(2,5-dichloro-3-(2-cyclopropyl-5-(2-(methylsulfonyl)pyrimidin-4-yl)thiazol-4-yl)phenyl)propane-1-sulfonamide (XXXIV, Scheme V, (1h)

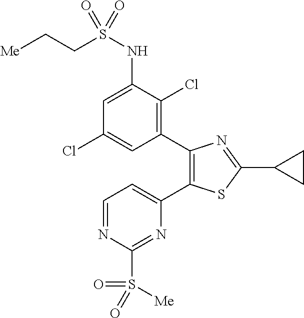 [SMILES: CCCS(=O)(=O)Nc1cc(Cl)cc(-c2nc(C3CC3)sc2-c2ccnc(S(C)(=O)=O)n2)c1Cl]

### Step 1. Preparation of N-(3-(2-amino-5-(2-(methylthio)pyrimidin-4-yl)thiazol-4-yl)-2,5-dichlorophenyl)pivalamide

The titled intermediate was obtained from N-(3-(2-bromo-2-(2-(methylthio)pyrimidin-4-yl)acetyl)-2,5-dichlorophenyl)propane-1-sulfonamide (intermediate XIX) using the procedure from intermediate XXIX, step 1: LCMS (m/z): 468 (MH⁺), t_{R}=0.92 min.

### Step 2. Preparation of N-(3-(2-bromo-5-(2-(methylthio)pyrimidin-4-yl)thiazol-4-yl)-2,5-dichlorophenyl)pivalamide

The titled intermediate was obtained from the above N-(3-(2-amino-5-(2-(methylthio)pyrimidin-4-yl)thiazol-4-yl)-2,5-dichlorophenyl)pivalamide using the procedure from intermediate XXIX, step 2: LCMS (m/z): 533/535 (MH⁺), t_{R}=1.34 min.

### Step 3. Preparation of N-(2,5-dichloro-3-(2-cyclopropyl-5-(2-(methylthio)pyrimidin-4-yl)thiazol-4-yl)phenyl)pivalamide

The titled intermediate was obtained from the above N-(3-(2-bromo-5-(2-(methylthio)pyrimidin-4-yl)thiazol-4-yl)-2,5-dichlorophenyl)pivalamide using the procedure from intermediate XXIX, step 3: LCMS (m/z): 493 (MH⁺), t_{R}=1.28 min.

### Step 4. Preparation of 2,5-dichloro-3-(2-cyclopropyl-5-(2-(methylthio)pyrimidin-4-yl)thiazol-4-yl)aniline

The titled intermediate was obtained from the above N-(2,5-dichloro-3-(2-cyclopropyl-5-(2-(methylthio)pyrimidin-4-yl)thiazol-4-yl)phenyl)pivalamide using the procedure from intermediate XXIX, step 4: LCMS (m/z): 409 (MH⁺), t_{R}=1.13 min.

### Step 5. Preparation of N-(2,5-dichloro-3-(2-cyclopropyl-5-(2-(methylthio)pyrimidin-4-yl)thiazol-4-yl)phenyl)propane-1-sulfonamide

The titled intermediate was obtained from the above 2,5-dichloro-3-(2-cyclopropyl-5-(2-(methylthio)pyrimidin-4-yl)thiazol-4-yl)aniline in a manner similar to intermediate IV, step 3 using 1-propanesulfonyl chloride: LCMS (m/z): 515 (MH⁺), t_{R}=1.17 min.

### Step 6. Preparation of N-(2,5-dichloro-3-(2-cyclopropyl-5-(2-(methylsulfonyl)pyrimidin-4-yl)thiazol-4-yl)phenyl)propane-1-sulfonamide

The titled intermediate was prepared from the above N-(2,5-dichloro-3-(2-cyclopropyl-5-(2-(methylthio)pyrimidin-4-yl)thiazol-4-yl)phenyl)propane-1-sulfonamide in a manner similar to intermediate XXXI, step 4: LCMS (m/z): 547 (MH⁺), t_{R}=0.96 min.

### Preparation of starting material 2-chloro-3-(2-cyclopropyl-5-(2-(methylthio)pyrimidin-4-yl)oxazol-4-yl)-5-fluoroaniline (XXXV, Scheme I, (1f)

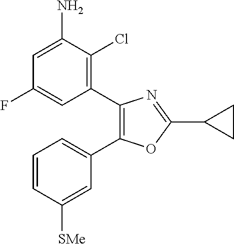 [SMILES: CSc1cccc(-c2oc(C3CC3)nc2-c2cc(F)cc(N)c2Cl)c1]

### Step 1. Preparation of N-(2-chloro-3-(2-cyclopropyl-5-(2-(methylthio)pyrimidin-4-yl)oxazol-4-yl)-5-fluorophenyl)pivalamide

To a solution of N-(3-(2-bromo-2-(2-(methylthio)pyrimidin-4-yl)acetyl)-2-chloro-5-fluorophenyl)pivalamide (XI, 400 mg, 0.84 mmol) in 1,3-dimethyl-3,4,5,6-tetrahydro-2(1H)-pyrimidinone (DMPU, 840 μL) at rt was added cyclopropanecarboxamide (1.43 g, 16.9 mmol). The reaction mixture was placed in a pre-heated 135° C. oil bath for 40 min, then allowed to cool to rt and partitioned between EtOAc and water. The aqueous phase was extracted with EtOAc and the organic layers were combined. The obtained organic layer was washed with brine and dried (Na₂SO₄), filtered and concentrated. The resulting residue was adsorbed onto silica gel and purified by flash chromatography on silica gel using an heptane-EtOAc (0-60%) elution gradient to afford N-(2-chloro-3-(2-cyclopropyl-5-(2-(methylthio)pyrimidin-4-yl)oxazol-4-yl)-5-fluorophenyl)pivalamide (170 mg, 0.365 mmol, 43% yield): LCMS (m/z): 461.2 (MH⁺), t_{R}=1.17 min.

### Step 2. Preparation of 2-chloro-3-(2-cyclopropyl-5-(2-(methylthio)pyrimidin-4-yl)oxazol-4-yl)-5-fluoroaniline

A solution of N-(2-chloro-3-(2-cyclopropyl-5-(2-(methylthio)pyrimidin-4-yl)oxazol-4-yl)-5-fluorophenyl)pivalamide (254 mg, 0.55 mmol) in ethanol (5.6 mL) was treated with an aqueous sulfuric acid solution (50% v/v, 2.9 mL). The resulting reaction mixture was heated in oil bath at 90° C. for 7 h. The reaction mixture was allowed to cool to rt and was then carefully added to a biphasic solution of EtOAc and saturated aqueous NaHCO₃ solution (basified to pH 7). The phases were separated and aqueous layer was extracted with EtOAc; the organics were combined, washed with brine, dried (Na₂SO₄), filtered and concentrated to afford 2-chloro-3-(2-cyclopropyl-5-(2-(methylthio)pyrimidin-4-yl)oxazol-4-yl)-5-fluoroaniline (191 mg, 0.46 mmol, 83% yield) as orange foam which contains 5-10% N-(2-chloro-3-(2-cyclopropyl-5-(2-(methylthio)pyrimidin-4-yl)oxazol-4-yl)-5-fluorophenyl)pivalamide: LCMS (m/z): 377.1 (MH⁺), t_{R}=0.95 min.

### Preparation of starting material 5-chloro-3-(2-cyclopropyl-5-(2-(methylthio)pyrimidin-4-yl)oxazol-4-yl)-2-fluoroaniline (XXXVI, Scheme I, (1f)

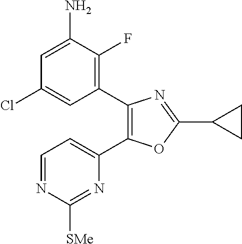 [SMILES: CSc1nccc(-c2oc(C3CC3)nc2-c2cc(Cl)cc(N)c2F)n1]

### Step 1. Preparation of N-(5-chloro-3-(2-cyclopropyl-5-(2-(methylthio)pyrimidin-4-yl)oxazol-4-yl)-2-fluorophenyl)pivalamide

A mixture of N-(3-(2-bromo-2-(2-(methylthio)pyrimidin-4-yl)acetyl)-5-chloro-2-fluorophenyl)-pivalamide (XII, 771 mg, 1.62 mmol), cyclopropanecarboxamide (2.763 mg, 32.5 mmol) and DMPU (1.6 mL) was heated in a pre-heated oil bath at 135° C. for 70 min. The reaction was then cooled to rt, partitioned between water (30 ml) and EtOAc (30 ml) and the layers separated. The organic layer was sequentially washed with saturated aqueous NaHCO₃ solution (20 ml), water (30 ml), brine (30 ml), dried (Na₂SO₄) and concentrated. The resulting residue was purified by flash chromatography on silica gel eluting with EtOAc-heptane (0-25%) to furnish N-(5-chloro-3-(2-cyclopropyl-5-(2-(methylthio)pyrimidin-4-yl)oxazol-4-yl)-2-fluorophenyl)pivalamide (773 mg) as a foam:

LCMS (m/z): 461.2 (MH⁺), t_{R}=1.14 min.

### Step 2. Preparation of 5-chloro-3-(2-cyclopropyl-5-(2-(methylthio)pyrimidin-4-yl)oxazol-4-yl)-2-fluoroaniline

A solution of N-(5-chloro-3-(2-cyclopropyl-5-(2-(methylthio)pyrimidin-4-yl)oxazol-4-yl)-2-fluorophenyl)pivalamide (748 mg) and H₂SO₄ (50% v/v, 6 ml) in EtOH (18 mL) was refluxed overnight, allowed to cool to rt, and concentrated. The resulting residue was diluted with ice water (30 mL), neutralized with excess solid NaHCO₃ and extracted with EtOAc (2×30 mL). The organic extracts were combined, washed with brine (30 mL), dried (Na₂SO₄), and concentrated. The resulting crude material was purified by flash chromatography on silica gel eluting with EtOAc-heptane (0-100%) to provide 5-chloro-3-(2-cyclopropyl-5-(2-(methylthio)pyrimidin-4-yl)oxazol-4-yl)-2-fluoroaniline (153 mg, 20% over two steps) as a yellow solid: LCMS (m/z): 377.1 (MH⁺), t_{R}=0.98 min.

### Preparation of starting material N-(3-(2-cyclopropyl-5-(2-(methylsulfonyl)pyrimidin-4-yl)oxazol-4-yl)-2-fluorophenyl)propane-1-sulfonamide (XXXVII, Scheme III, (1h)

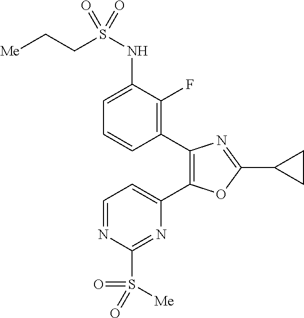 [SMILES: CCCS(=O)(=O)Nc1cccc(-c2nc(C3CC3)oc2-c2ccnc(S(C)(=O)=O)n2)c1F]

### Step 1. Preparation of N-(3-(2-cyclopropyl-5-(2-(methylthio)pyrimidin-4-yl)oxazol-4-yl)-2-fluorophenyl)propane-1-sulfonamide

The titled intermediate was obtained from N-(3-(2-bromo-2-(2-(methylthio)pyrimidin-4-yl)acetyl)-2-fluorophenyl)propane-1-sulfonamide (intermediate XIII) using the procedure from intermediate XXXV, step 1: LCMS (m/z): 449.2 (MH⁺), t_{R}=0.95 min; ¹H NMR (400 MHz, CDCl₃) δ ppm 1.02 (t, J=7.4Hz, 3H), 1.15-1.32 (m, 4H), 1.87 (m, 2H), 2.11 (s, 3H), 2.17-2.26 (m, 1H), 3.09 (m, 2H), 6.54 (d, J=2.4Hz, 1H), 7.18 (d, J=5.1 Hz, 1H), 7.23 (t, J=7.2 Hz, 1H), 7.34-7.41 (m, 1H), 7.68 (t, J=7.2 Hz, 1H), 8.52 (d, J=5.1, Hz, 1H).

### Step 2. Preparation of N-(3-(2-cyclopropyl-5-(2-(methylsulfonyl)pyrimidin-4-yl)oxazol-4-yl)-2-fluorophenyl)propane-1-sulfonamide

The titled intermediate was prepared from N-(3-(2-cyclopropyl-5-(2-(methylthio)pyrimidin-4-yl)oxazol-4-yl)-2-fluorophenyl)propane-1-sulfonamide using the procedure for intermediate XXXI, step 4 to provide a mixture of sulfoxide and the desired sulfone: LCMS (m/z): 465.2 (sulfoxide MH⁺), t_{R}=0.72 min LCMS (m/z): 481.2 (sulfone MH⁺), t_{R}=0.80 min.

### Preparation of starting material N-(2-chloro-3-(2-cyclopropyl-5-(2-(methylsulfonyl)pyrimidin-4-yl)oxazol-4-yl)phenyl)propane-1-sulfonamide (XXXVIII, Scheme III, (1h)

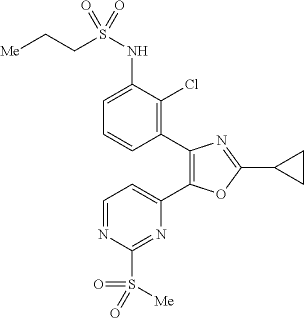 [SMILES: CCCS(=O)(=O)Nc1cccc(-c2nc(C3CC3)oc2-c2ccnc(S(C)(=O)=O)n2)c1Cl]

### Step 1. Preparation of N-(2-chloro-3-(2-cyclopropyl-5-(2-(methylthio)pyrimidin-4-yl)oxazol-4-yl)phenyl)pivalamide

The titled intermediate was prepared from N-(3-(2-bromo-2-(2-(methylthio)pyrimidin-4-yl)acetyl)-2-chlorophenyl)pivalamide (intermediate XVII), according to the procedure for intermediate XXXV, step 1: LCMS (m/z): 443.3 (MH⁺), t_{R}=1.07 min; ¹H NMR (400 MHz, CDCl₃) δ ppm 1.16-1.23 (m, 2H) 1.24-1.31 (m, 2H) 1.35 (s, 9H) 1.94 (s, 3H) 2.17-2.25 (m, 1H) 7.10 (d, J=5.09 Hz, 1H) 7.16-7.20 (m, 1H) 7.34 (t, J=8.02 Hz, 1H) 8.12 (s, 1H) 8.45 (d, J=5.09 Hz, 1H) 8.50 (dd, J=8.22, 1.57 Hz, 1H).

### Step 2. 2-chloro-3-(2-cyclopropyl-5-(2-(methylthio)pyrimidin-4-yl)oxazol-4-yl)aniline

The titled intermediate was obtained from material above according to the procedure for intermediate XXXV, step 2: LCMS (m/z): 359.1 (MH⁺), t_{R}=0.89 min; ¹H NMR (400 MHz, CDCl₃) δ ppm 1.13-1.22 (m, 2H) 1.23-1.30 (m, 3H) 2.05 (s, 3H) 2.14-2.26 (m, 1H) 4.14 (s, 2H) 6.73-6.88 (m, 2H) 7.06 (d, J=5.09 Hz, 1H) 7.12 (t, J=7.83 Hz, 1H) 8.42 (d, J=5.09 Hz, 1H).

### Step 3. N-(2-chloro-3-(2-cyclopropyl-5-(2-(methylthio)pyrimidin-4-yl)oxazol-4-yl)phenyl)propane-1-sulfonamide

The titled intermediate was obtained from the material above following the procedure for intermediate IV, step 3 using 1-propanesulfonyl chloride: LCMS (m/z): 465.1 (MH⁺), t_{R}=0.97 min; ¹H NMR (400 MHz, CDCl₃) δ ppm 1.04 (t, J=7.4Hz, 3H) 1.14-1.32 (m, 4H) 1.80-1.92 (m, 2H) 1.95 (s, 3H) 2.14-2.26 (m, 1H) 3.04-3.16 (m, 2H) 7.13 (d, J=5.1 Hz, 1H) 7.22-7.28 (m, 1H) 7.36 (t, J=7.8 Hz, 1H) 7.76 (dd, J=8.2, 1.6 Hz, 1H) 8.48 (d, J=5.1 Hz, 1H).

### Step 4. N-(2-chloro-3-(2-cyclopropyl-5-(2-(methylsulfonyl)pyrimidin-4-yl)oxazol-4-yl)phenyl)propane-1-sulfonamide

The titled intermediated was prepared from the above N-(2-chloro-3-(2-cyclopropyl-5-(2-(methylthio)pyrimidin-4-yl)oxazol-4-yl)phenyl)propane-1-sulfonamide using the procedure from intermediate XXXI, step 4: LCMS (m/z): 497.1 (MH⁺), t_{R}=0.81 min.

### Preparation of starting material N-(2,5-dichloro-3-(2-cyclopropyl-5-(2-(methylsulfonyl)pyrimidin-4-yl)oxazol-4-yl)phenyl)propane-1-sulfonamide (XXXIX, Scheme III, (1h)

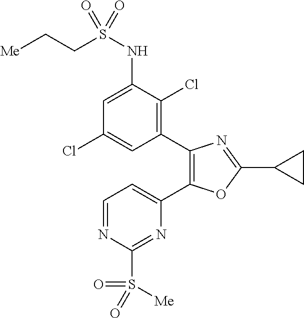 [SMILES: CCCS(=O)(=O)Nc1cc(Cl)cc(-c2nc(C3CC3)oc2-c2ccnc(S(C)(=O)=O)n2)c1Cl]

### Step 1. Preparation of N-(2,5-dichloro-3-(2-cyclopropyl-5-(2-(methylthio)pyrimidin-4-yl)oxazol-4-yl)phenyl)pivalamide

The compound was prepared from N-(3-(2-bromo-2-(2-(methylthio)pyrimidin-4-yl)acetyl)-2,5-dichlorophenyl)pivalamide (intermediate XVII), according to the procedure for intermediate XXXV, step 1: LCMS (m/z): 477 (MH⁺), t_{R}=1.20 min.

### Step 2. Preparation of 2,5-dichloro-3-(2-cyclopropyl-5-(2-(methylthio)pyrimidin-4-yl)oxazol-4-yl)aniline

The compound was prepared from the above N-(2,5-dichloro-3-(2-cyclopropyl-5-(2-(methylthio)pyrimidin-4-yl)oxazol-4-yl)phenyl)pivalamide according to the procedure for intermediate XXXV, step 2: LCMS (m/z): 393 (MH⁺), t_{R}=1.02 min.

### Step 3. Preparation of N-(2,5-dichloro-3-(2-cyclopropyl-5-(2-(methylthio)pyrimidin-4-yl)oxazol-4-yl)phenyl)propane-1-sulfonamide

The compound was prepared from 2,5-dichloro-3-(2-cyclopropyl-5-(2-(methylthio)pyrimidin-4-yl)oxazol-4-yl)aniline in a manner similar to intermediate IV, step 4 using 1-propanesulfonyl chloride: LCMS (m/z): 499 (MH⁺), t_{R}=1.07 min.

### Step 4. Preparation of N-(2,5-dichloro-3-(2-cyclopropyl-5-(2-(methylsulfonyl)pyrimidin-4-yl)oxazol-4-yl)phenyl)propane-1-sulfonamide

The compound was prepared from the above N-(2,5-dichloro-3-(2-cyclopropyl-5-(2-(methylthio)pyrimidin-4-yl)oxazol-4-yl)phenyl)propane-1-sulfonamide using the procedure for intermediate XXXI, step 4: LCMS (m/z): 531 (MH⁺), t_{R}=0.89 min.

### Example 1

### Preparation of (S)-methyl 1-(4-(2-tert-butyl-4-(2-chloro-5-fluoro-3-(methylsulfonamido)phenyl)thiazol-5-yl)pyrimidin-2-ylamino)propan-2-ylcarbamate

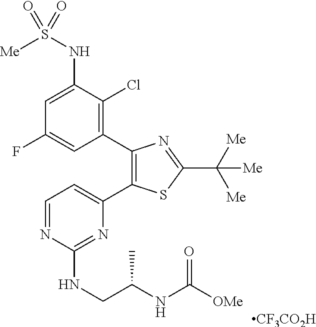 [SMILES: COC(=O)N[C@@H](C)CNc1nccc(-c2sc(C(C)(C)C)nc2-c2cc(F)cc(NS(C)(=O)=O)c2Cl)n1 ; "·CF3CO2H"]

A mixture of 3-(2-tert-butyl-5-(2-(methylthio)pyrimidin-4-yl)thiazol-4-yl)-2-chloro-5-fluoroaniline (XXI, 104 mg, 0.25 mmol), pyridine (1 mL), and methanesulfonyl chloride (0.079 mL, 1.02 mmol) was stirred for 18 h at rt, concentrated and the resulting residue was suspended in a mixture of DME (5 ml) and saturated aqueous Na₂CO₃ solution (5 mL). The biphasic reaction mixture was heated to 65° C. for 2 h. The reaction mixture was cooled to room temperature, diluted with water and extracted with EtOAc twice. The organics were combined, washed with water, brine, dried (Na₂SO₄), filtered and concentrated to give N-(3-(2-tert-butyl-5-(2-(methylthio)-pyrimidin-4-yl)thiazol-4-yl)-2-chloro-5-fluorophenyl)methanesulfonamide (131 mg, 0.24 mmol, 95%) as a brown residue which was used without further purification: LCMS (m/z): 487.0 (MH⁺), t_{R}=1.20 min.

N-(3-(2-tert-butyl-5-(2-(methylthio)-pyrimidin-4-yl)thiazol-4-yl)-2-chloro-5-fluorophenyl)methanesulfonamide (131 mg, 0.269 mmol) was dissolved in DCM (2.7 mL) under nitrogen. The mixture was cooled to 0° C. in an ice/water bath and 60% mCPBA (155 mg, 0.54 mmol) was added. The resulting reaction mixture was allowed to stir for 20 min at 0° C., allowed to warm to rt and quenched with saturated aqueous NaHCO₃ solution (pH of resulting water phase was 7-8). The aqueous phase was extracted with EtOAc twice. The organic extracts were combined, washed with water, brine, dried (Na₂SO₄), filtered and concentrated to afford N-(3-(2-tert-butyl-5-(2-(methylsulfonyl)pyrimidin-4-yl)thiazol-4-yl)-2-chloro-5-fluorophenyl)methanesulfonamide (107 mg, 0.196 mmol) as a viscous yellow oil which crystallized upon standing: LCMS (m/z): 519.1 (MH⁺), t_{R}=0.94 min.

A solution of N-(3-(2-tert-butyl-5-(2-(methylsulfonyl)pyrimidin-4-yl)thiazol-4-yl)-2-chloro-5-fluorophenyl)methanesulfonamide (36 mg, 0.069 mmol) and (S)-tert-butyl 1-aminopropan-2-ylcarbamate (X, 121 mg, 0.69 mmol) in NMP (1 mL) was stirred for 15 min at rt then heated to 120° C. for 15 min. The reaction mixture was allowed to cool to rt, diluted with a saturated aqueous NH₄Cl solution and extracted with EtOAc twice. The organics were combined, washed with brine, dried (Na₂SO₄), filtered and concentrated to afford (S)-tert-butyl 1-(4-(2-tert-butyl-4-(2-chloro-5-fluoro-3-(methylsulfonamido)phenyl)thiazol-5-yl)pyrimidin-2-ylamino)propan-2-ylcarbamate (40 mg, 0.065 mmol, 94%) as a yellow oil: LCMS (m/z): 613.3 (MH⁺), t_{R}=0.99 min.

To a round bottom flask containing (S)-tert-butyl 1-(4-(2-tert-butyl-4-(2-chloro-5-fluoro-3-(methylsulfonamido)phenyl)thiazol-5-yl)pyrimidin-2-ylamino)propan-2-ylcarbamate (40 mg, 0.065 mmol) was added a 1:1 solution of TFA:DCM (1 mL). The resulting reaction was stirred for 10 min at rt, was then concentrated and suspended in a mixture of THF (1 mL) and a saturated aqueous NaHCO₃ solution (1 mL). To this biphasic solution was added methyl chloroformate (6 μL, 0.072 mmol) as a 0.1 M solution in THF. The biphasic reaction mixture was stirred rapidly for 15 min at rt. LCMS of organic layer indicated little conversion. Additional methyl chloroformate (15 μL) was then added, and the reaction mixture was stirred rapidly for another 15 min. LCMS of a reaction aliquot indicated complete conversion. The reaction mixture was diluted with water, extracted with EtOAc twice; the organic phases were combined, washed with brine, dried (Na₂SO₄), filtered and concentrated. The residue was dissolved in DMSO and purified by reverse phase preparative HPLC. Product fractions were combined and lyophilized to afford (S)-methyl 1-(4-(2-tert-butyl-4-(2-chloro-5-fluoro-3-(methylsulfonamido)phenyl)thiazol-5-yl)pyrimidin-2-ylamino)propan-2-ylcarbamate as the TFA salt (14 mg):

LCMS (m/z): 571.1 (MH⁺), t_{R}=0.86 min.

### Example 2

### Preparation of N-(3-(2-tert-butyl-5-(pyrimidin-4-yl)thiazol-4-yl)-2-chloro-5-fluorophenyl)propane-1-sulfonamide

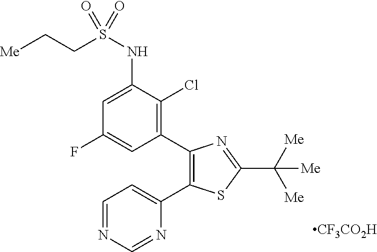 [SMILES: CCCS(=O)(=O)Nc1cc(F)cc(-c2nc(C(C)(C)C)sc2-c2ccncn2)c1Cl ; "·CF3CO2H"]

To a solution of 3-(2-tert-butyl-5-(2-(methylsulfonyl)pyrimidin-4-yl)thiazol-4-yl)-2-chloro-5-fluoroaniline (XX/, 30 mg, 0.073 mmol) in pyridine (0.4 mL), 1-propanesulfonyl chloride (33 μL, 0.29 mmol) was added. The reaction mixture was allowed to stir 18 h at rt. LCMS of an aliquot indicated that the reaction was incomplete. Additional 1-propanesulfonyl chloride (32.9 μL, 0.293 mmol) was added. The reaction mixture was stirred for an additional 72 h at rt, quenched with saturated aqueous NaHCO₃ solution (pH=8) and extracted with EtOAc. The aqueous phase was then adjusted to pH 6 with aqueous 1 N HCl solution and was extracted a second time with EtOAc. The organic extracts were combined, washed with water, brine, dried (Na₂SO₄), filtered and concentrated to afford crude N-(3-(2-tert-butyl-5-(2-(methylthio)pyrimidin-4-yl)thiazol-4-yl)-2-chloro-5-fluorophenyl)propane-1-sulfonamide (39 mg, 0.068 mmol) as a brown solid, which was used in the next step without further purification: LCMS (m/z): 515.1 (MH⁺), t_{R}=1.30 min.

N-(3-(2-tert-butyl-5-(2-(methylthio)pyrimidin-4-yl)thiazol-4-yl)-2-chloro-5-fluoro phenyl)propane-1-sulfonamide (39 mg, 0.076 mmol) was dissolved in DCM (2 mL) and the solution was cooled to 0° C. in a ice-water bath. To the cooled reaction was added mCPBA (50%, 52 mg, 0.15 mmol), the resulting reaction mixture was allowed to stir 20 min at 0° C. then quenched with saturated aqueous NaHCO₃ solution. The aqueous phase was extracted twice with EtOAc. The organic extracts were combined, washed with water, brine, dried (Na₂SO₄), filtered and concentrated to afford N-(3-(2-tert-butyl-5-(2-(methylsulfonyl)pyrimidin-4-yl)thiazol-4-yl)-2-chloro-5-fluorophenyl)propane-1-sulfonamide (25 mg, 0.046 mmol, 60%) as a viscous yellow residue which crystallized upon standing: LCMS (m/z): 547.0 (MH⁺), t_{R}=0.99 min.

To a solution of N-(3-(2-tert-butyl-5-(2-(methylsulfonyl)pyrimidin-4-yl)thiazol-4-yl)-2-chloro-5-fluorophenyl)propane-1-sulfonamide (25 mg, 0.046 mmol) in DCM (0.5 mL) and EtOH (0.5 mL) was added sodium borohydride (3.5 mg, 0.091 mmol). The resulting reaction mixture was stirred at ambient temperature for 18 h under nitrogen. More sodium borohydride (12 mg, 0.32 mmol) was then added and the reaction mixture was allowed to stir an additional 18 h. The reaction was quenched with water and extracted twice with EtOAc. The organic extracts were combined, washed with water, brine, dried (Na₂SO₄), filtered and concentrated. The residue was dissolved in DMSO and purified by reverse phase preparative HPLC. Fractions containing product were combined, frozen and lyophilized to afford N-(3-(2-tert-butyl-5-(pyrimidin-4-yl)thiazol-4-yl)-2-chloro-5-fluorophenyl)propane-1-sulfonamide (2 mg) as the TFA salt. LCMS (m/z): 469.1, (MH⁺), t_{R}=1.08 min.

### Example 3

### Preparation of (S)-methyl 1-(4-(4-(2-chloro-5-fluoro-3-(methylsulfonamido)phenyl)-2-cyclopropylthiazol-5-yl)pyrimidin-2-ylamino)propan-2-ylcarbamate

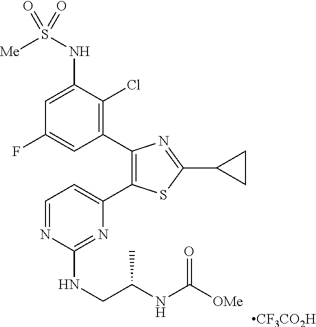 [SMILES: COC(=O)N[C@@H](C)CNc1nccc(-c2sc(C3CC3)nc2-c2cc(F)cc(NS(C)(=O)=O)c2Cl)n1 ; "·CF3CO2H"]

A solution of 2-chloro-3-(2-cyclopropyl-5-(2-(methylthio)-pyrimidin-4-yl)thiazol-4-yl)-5-fluoroaniline (XXIX, 86 mg, 0.219 mmol) in dry pyridine (440 μL) was treated with methanesulfonyl chloride (85 μL, 1.1 mmol) at rt and the reaction was stirred for 3 h. LCMS of an aliquot indicated 90% conversion as a mixture of sulfonamide and sulfonimide. The reaction was stirred for additional 2 h, concentrated and the resulting residue was partitioned between DME (5 mL) and saturated aqueous Na₂CO₃ solution (5 mL). The biphasic reaction mixture was heated to 60° C. for 1 h. LCMS indicated complete conversion to the desired sulfonamide. The reaction was allowed to cool to rt, diluted with water and extracted with EtOAc twice. The organics were combined, washed with water, brine, dried (Na₂SO₄), filtered and concentrated. The resulting residue was adsorbed onto silica gel and purified by flash chromatography on silica gel using an EtOAc-heptane (0-100%) elution gradient. The product fractions were combined and concentrated to afford N-(2-chloro-3-(2-cyclopropyl-5-(2-(methylthio)pyrimidin-4-yl)thiazol-4-yl)-5-fluorophenyl)methanesulfonamide (59 mg, 0.12 mmol, 57% yield) as a light brown crystalline solid: LCMS (m/z): 471.1 (MH⁺), t_{R}=1.03 min; ¹H NMR (300 MHz, CDCl₃) δ ppm 1.15-1.34 (m, 4H) 2.33-2.43 (m, 1H) 2.47 (s, 3H) 3.11 (s, 3H) 6.47 (d, J=5.3 Hz, 1H) 6.98-7.05 (m, 1H) 7.59 (dd, J=9.4, 2.9 Hz, 1H) 8.31 (d, J=5.3 Hz, 1H).

A solution of N-(2-chloro-3-(2-cyclopropyl-5-(2-(methylthio)pyrimidin-4-yl)thiazol-4-yl)-5-fluorophenyl)methanesulfonamide (58 mg, 0.12 mmol) in DCM (1.2 mL) under a nitrogen atmosphere was treated with 60% mCPBA (71 mg, 0.25 mmol). The resulting reaction mixture was stirred at rt for 2 h. LCMS indicated complete conversion and the reaction mixture was quenched with saturated aqueous NaHCO₃ solution and extracted with EtOAc twice. The organic phases were combined, washed with water, brine, dried (Na₂SO₄), and concentrated to afford N-(2-chloro-3-(2-cyclopropyl-5-(2-(methylsulfonyl)pyrimidin-4-yl)thiazol-4-yl)-5-fluorophenyl)-methanesulfonamide (65 mg, 0.129 mmol) as a viscous yellow residue which was carried to the next step without further purification: LCMS (m/z): 503.2 (MH⁺), t_{R}=0.81 min.

A solution of N-(2-chloro-3-(2-cyclopropyl-5-(2-(methylsulfonyl)pyrimidin-4-yl)thiazol-4-yl)-5-fluorophenyl)methane-sulfonamide (32 mg, 0.064 mmol) and (S)-tert-butyl 1-aminopropan-2-ylcarbamate (X, 111 mg, 0.64 mmol) in NMP (1 ml) was irradiated to 120° C. for 10 min in a microwave reactor. LCMS indicated complete conversion and the reaction was diluted with a saturated aqueous solution of NH₄Cl and twice extracted with EtOAc. The combined organics were washed with brine, dried (Na₂SO₄), and concentrated to give (S)-tert-butyl-1-(4-(4-(2-chloro-5-fluoro-3-(methylsulfonamido)phenyl)-2-cyclopropylthiazol-5-yl)pyrimidin-2-ylamino)propan-2-ylcarbamate (43 mg, 0.065 mmol) as a yellow oil which was carried to the next step without further purification: LCMS (m/z): 597.4 (MH⁺), t_{R}=0.88 min.

(S)-tert-butyl-1-(4-(4-(2-chloro-5-fluoro-3-(methylsulfonamido)phenyl)-2-cyclopropylthiazol-5-yl)pyrimidin-2-ylamino)-propan-2-ylcarbamate (43 mg, 0.072 mmol) was treated with solution of TFA in DCM (50% v/v, 1 mL) at rt for 10 min, The reaction mixture was then concentrated and the resulting residue was dissolved in THF (2 mL) and a saturated aqueous solution of NaHCO₃ (2 mL) forming a biphasic mixture. Methyl chloroformate (0.028 mL, 0.360 mmol) was added and the resulting reaction mixture was stirred rapidly for 15 min at rt. LCMS indicated complete conversion and the reaction mixture was diluted with water, and extracted twice with EtOAc. The organic phases were combined, washed with brine, dried (Na₂SO₄), and concentrated to a yellow residue which was purified by preparative reverse phase HPLC. The fractions containing product were combined, and lyophilized to afford (S)-methyl 1-(4-(4-(2-chloro-5-fluoro-3-(methylsulfonamido)phenyl)-2-cyclopropylthiazol-5-yl)pyrimidin-2-ylamino)propan-2-ylcarbamate (12 mg, 0.018 mmol) as the TFA salt: LCMS (m/z): 555.2 (MH⁺), t_{R}=0.74 min; ¹H NMR (300 MHz, acetic acid-d⁴) δ 1.15-1.27 (m, 2H) 1.20 (d, J=6.7 Hz, 3H) 2.50-2.64 (m, 1H) 3.13 (s, 3H) 3.22-3.46 (m, 1H) 3.61 (s, 3H) 3.69 (m, 1H), 3.89-4.12 (m, 1H) 6.41 (d, J=5.9 Hz, 1H) 7.19 (dd, J=8.2, 2.9 Hz, 1H) 7.62 (dd, J=9.7, 2.9 Hz, 1H) 8.21 (d, J=6.2 Hz, 1H).

### Example 4

### Preparation of (S)-methyl 1-(4-(4-(5-chloro-2-fluoro-3-(methylsulfonamido)phenyl)-2-cyclopropylthiazol-5-yl)pyrimidin-2-ylamino)propan-2-ylcarbamate

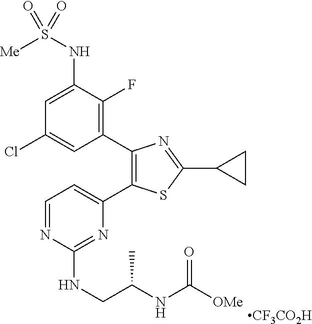 [SMILES: COC(=O)N[C@@H](C)CNc1nccc(-c2sc(C3CC3)nc2-c2cc(Cl)cc(NS(C)(=O)=O)c2F)n1 ; "·CF3CO2H"]

A solution of 5-chloro-3-(2-cyclopropyl-5-(2-(methylthio)pyrimidin-4-yl)thiazol-4-yl)-2-fluoroaniline (XXX, 113 mg, 0.29 mmol) and anhydrous pyridine (0.23 mL, 2.86 mmol) in DCM (5 mL) was treated with methanesulfonyl chloride (0.18 mL, 2.29 mmol) and the resulting reaction mixture was stirred at rt for 21 h. LCMS indicated complete conversion to the sulfonamide and a small amount of the sulfonimide. The reaction was quenched with water (100 uL) and concentrated. The resulting crude residue was suspended in DME (15 mL) and saturated aqueous Na₂CO₃ solution (5 mL) and the resulting mixture was heated at 60° C. for 2 h with vigorous stirring. LCMS indicated complete conversion to the sulfonamide. The reaction was allowed to cool to rt and the resulting partitioned layers were separated. The organic layer was collected, and the remaining suspension was filtered and the collected solids were washed with MeOH (2×10 mL). The filtrates were combined with the DME layer and concentrated. The resulting residue was partitioned between EtOAc (30 mL) and 0.1 N sodium phosphate buffer (pH 7.0, 30 mL) and the layers were separated. The organic portion was washed with brine (30 mL), dried (Na₂SO₄), concentrated to give N-(5-chloro-3-(2-cyclopropyl-5-(2-(methylthio)pyrimidin-4-yl)thiazol-4-yl)-2-fluorophenyl)methanesulfonamide as a yellow residue (134 mg) which was used without further purification: LCMS (m/z): 471.1 (MH⁺), t_{R}=1.06 min.

A solution of N-(5-chloro-3-(2-cyclopropyl-5-(2-(methylthio)pyrimidin-4-yl)thiazol-4-yl)-2-fluorophenyl)methanesulfonamide (134 mg, 0.28 mmol) in DCM (10 mL) was treated with 70% mCPBA (70 mg, 0.28 mmol) at rt for 15 min. The reaction mixture was then washed with 0.1 N sodium phosphate buffer (pH 7, 4×20 ml), brine (10 ml), dried (Na₂SO₄), concentrated to give N-(5-chloro-3-(2-cyclopropyl-5-(2-(methylsulfinyl)pyrimidin-4-yl)thiazol-4-yl)-2-fluorophenyl)methanesulfonamide (120 mg) as a yellow residue which was used without further purification: LCMS (m/z): 487.1 (MH t_{R}=0.77 min.

A solution of N-(5-chloro-3-(2-cyclopropyl-5-(2-(methylsulfonyl)pyrimidin-4-yl)thiazol-4-yl)-2-fluorophenyl)methanesulfonamide (50 mg, 0.103 mmol) and (S)-tert-butyl 1-aminopropan-2-ylcarbamate (X, 39 mg, 0.22 mmol) in NMP (1 mL) was heated at 100° C. for 2 h. The reaction mixture was cooled to rt, diluted with EtOAc (3 ml) and washed with 0.1 N sodium phosphate buffer (pH 7, 3×3 mL). The EtOAc extracts were combined, washed with brine (3 ml), dried (Na₂SO₄), concentrated to provide (S)-tert-butyl 1-(4-(4-(5-chloro-2-fluoro-3-(methylsulfonamido)phenyl)-2-cyclopropylthiazol-5-yl)pyrimidin-2-ylamino)propan-2-ylcarbamate (65 mg) as a yellow foam which was carried forward without further purification: LCMS (m/z): 597.4 (MH⁺), t_{R}=0.91 min.

A solution of (S)-tert-butyl 1-(4-(4-(5-chloro-2-fluoro-3-(methylsulfonamido)phenyl)-2-cyclopropylthiazol-5-yl)pyrimidin-2-ylamino)propan-2-ylcarbamate (65 mg, 0.103 mmol) in MeOH (1 mL) was treated with conc. HCl (100 uL) at rt for 3 h, then at 60° C. for 1 h, and finally allowed to cool to rt and concentrated to dryness. The resulting brown residue was partitioned between THF (3 mL) and saturated aqueous NaHCO₃ solution (3 mL). The biphasic reaction mixture was treated with methyl chloroformate (8 uL, 0.10 mmol) stirring for 5 min at rt. The reaction mixture was extracted with EtOAc (3 mL). The organic layer was washed with brine (2×3 mL), dried (Na₂SO₄) and concentrated. The resulting crude residue was purified by reverse phase preparative HPLC. The pure fractions were combined and lyophilized to give (S)-methyl 1-(4-(4-(5-chloro-2-fluoro-3-(methylsulfonamido)phenyl)-2-cyclopropylthiazol-5-yl)pyrimidin-2-ylamino)propan-2-ylcarbamate as the TFA salt (23 mg):

LCMS (m/z): 555.1 (MH⁺), t_{R}=0.80 min; ¹H NMR (300 MHz, acetic acid-d⁴) δ ppm 1.17 (d, J=6.5 Hz, 3H), 1.20-1.28 (m, 2H), 1.28-1.46 (m, 2H), 2.47-2.65 (m, 1H), 3.11 (s, 3H), 3.19-3.37 (m, 1H), 3.60 (s, 3H), 3.64-3.76 (m, 1H), 3.95 (br. s., 1H), 6.60-6.78 (m, 1H), 7.41-7.53 (m, 1H), 7.68-7.80 (m, 1H), 8.26-8.36 (m, 1H).

### Example 5

### Preparation of (S)-methyl 1-(4-(4-(5-chloro-2-fluoro-3-(methylsulfonamido)phenyl)-2-cyclopropyloxazol-5-yl)pyrimidin-2-ylamino)propan-2-ylcarbamate

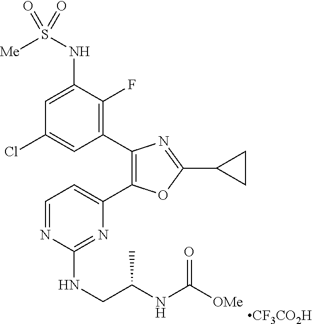 [SMILES: COC(=O)N[C@@H](C)CNc1nccc(-c2oc(C3CC3)nc2-c2cc(Cl)cc(NS(C)(=O)=O)c2F)n1 ; "·CF3CO2H"]

A solution of 5-chloro-3-(2-cyclopropyl-5-(2-(methylthio)pyrimidin-4-yl)oxazol-4-yl)-2-fluoroaniline (XXXVI, 153 mg, 0.41 mmol) and anhydrous pyridine (0.33 mL, 4.07 mmol) in DCM (5 mL) was treated with methanesulfonyl chloride (0.25 mL, 3.25 mmol) and the resulting reaction was stirred at rt overnight. LCMS indicated complete conversion to the sulfonamide and a small amount of the sulfonimide. The reaction was quenched with water (100 uL) and concentrated. The crude residue was partitioned between DME (15 mL) and saturated aqueous Na₂CO₃ solution (5 mL) and heated at 60° C. for 2 h. The reaction was allowed to cool to rt and the resulting partitioned layers were separated. The organic layer was collected, and the remaining suspension was filtered and the collected solids were washed with MeOH (2×10 mL). The filtrates were combined with the DME layer and concentrated. The resulting residue was partitioned between EtOAc (30 mL) and 0.1 N sodium phosphate buffer (pH 7.0, 30 mL). The EtOAc extracts were washed with brine (30 mL), dried (Na₂SO₄), concentrated to furnish N-(5-chloro-3-(2-cyclopropyl-5-(2-(methylthio)pyrimidin-4-yl)oxazol-4-yl)-2-fluorophenyl)methane-sulfonamide as a yellow residue (166 mg) which was used without further purification: LCMS (m/z): 455.0 (MH⁺), t_{R}=0.94 min.

A solution of N-(5-chloro-3-(2-cyclopropyl-5-(2-(methylthio)pyrimidin-4-yl)oxazol-4-yl)-2-fluorophenyl)methane-sulfonamide (166 mg, 0.37 mmol) in DCM (25 mL) was treated with 70% mCPBA (90 mg, 0.37 mmol) at rt for 10 min. The reaction mixture was concentrated and the crude residue was partitioned between EtOAc (30 mL) and 0.1 N sodium phosphate buffer (pH 7, 30 mL). The layers were separated and the organic portion was washed with 0.1 N aqueous sodium phosphate buffer (pH 7.0, 2×30 mL), brine (30 mL), dried (Na₂SO₄), and concentrated to give N-(5-chloro-3-(2-cyclopropyl-5-(2-(methylsulfinyl)pyrimidin-4-yl)oxazol-4-yl)-2-fluorophenyl)methanesulfonamide (160 mg, 93% yield) as a yellow residue which was used without further purification: LCMS (m/z): 471.1 (MH⁺), t_{R}=0.72 min.

A solution of N-(5-chloro-3-(2-cyclopropyl-5-(2-(methylsulfinyl)pyrimidin-4-yl)oxazol-4-yl)-2-fluorophenyl)methanesulfonamide (50 mg, 0.106 mmol) and (S)-tert-butyl 1-aminopropan-2-ylcarbamate (X, 41 mg, 0.234 mmol) in NMP (1 mL) was heated at 100° C. for 2 h. The reaction was allowed to cool to rt, diluted with EtOAc (3 mL) and washed with 0.1 N aqueous sodium phosphate buffer (pH 7, 3×3 mL), brine (3 mL), dried (Na₂SO₄), concentrated to provide (S)-tert-butyl 1-(4-(4-(5-chloro-2-fluoro-3-(methylsulfonamido)phenyl)-2-cyclopropyloxazol-5-yl)pyrimidin-2-ylamino)propan-2-ylcarbamate (63 mg) as a yellow foam which was carried forward without further purification: LCMS (m/z): 581.4 (MH⁺), t_{R}=0.84 min.

A solution of (S)-tert-butyl 1-(4-(4-(5-chloro-2-fluoro-3-(methylsulfonamido)phenyl)-2-cyclopropyloxazol-5-yl)pyrimidin-2-ylamino)propan-2-ylcarbamate (63 mg, 0.11 mmol) in MeOH (1 mL) was treated with conc. HCl (100 uL) at room temperature for 3 h, then at 60° C. for 1 h. The reaction mixture was cooled to rt and concentrated to dryness. The resulting residue was partitioned between THF (3 mL) and saturated aqueous NaHCO₃ solution (3 mL). The biphasic reaction mixture was treated with methyl chloroformate (8 uL, 0.11 mmol) for 10 min at rt with vigorous stirring. The reaction mixture was extracted with EtOAc (3 ml), the organic layer was washed with brine (2×3 mL), dried (Na₂SO₄) and concentrated. The resulting residue was purified by preparative reverse-phase HPLC and pure fractions were combined and lyophilized to give (S)-methyl 1-(4-(4-(5-chloro-2-fluoro-3-(methylsulfonamido)phenyl)-2-cyclopropyloxazol-5-yl)pyrimidin-2-ylamino)propan-2-ylcarbamate as the TFA salt (27 mg):

LCMS (m/z): 539.1 (MH⁺), t_{R}=0.72 min; ¹H NMR (300 MHz, acetic acid-d⁴) δ ppm 0.83-1.10 (m, 3H), 1.22-1.41 (m, 4H), 2.30-2.48 (m, 1H), 2.92-3.08 (m, 1H), 3.13 (s, 3H) 3.17-3.33 (m, 1H), 3.58 (s, 3H), 3.64-3.79 (m, 1H), 7.02-7.25 (m, 1H), 7.45-7.57 (m, 1H), 7.67-7.82 (m, 1H), 8.42-8.55 (m, 1H).

### Example 6

### Preparation of N-(3-(2-cyclopropyl-5-(2-(methylamino)pyrimidin-4-yl)oxazol-4-yl)-2-fluorophenyl)propane-1-sulfonamide

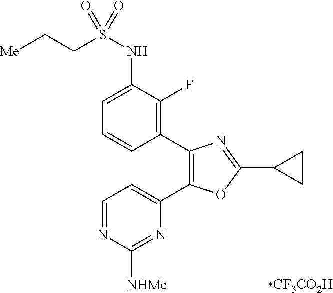 [SMILES: CCCS(=O)(=O)Nc1cccc(-c2nc(C3CC3)oc2-c2ccnc(NC)n2)c1F ; "·CF3CO2H"]

A solution of N-(3-(2-cyclopropyl-5-(2-(methylsulfonyl)pyrimidin-4-yl)oxazol-4-yl)-2-fluorophenyl)propane-1-sulfonamide (XXXVII, 20 mg, 0.042 mmol) and methylamine (40% aq, 130 μL, 4.2 mmol) was heated to 90° C. for 4 h. The reaction mixture was concentrated, the resulting residue dissolved in DMSO and purified by reverse phase preparative HPLC. The fractions containing pure product were collected and lyophilized to furnish N-(3-(2-cyclopropyl-5-(2-(methylamino)pyrimidin-4-yl)oxazol-4-yl)-2-fluorophenyl)propane-1-sulfonamide (5 mg) as the TFA salt:

LCMS (m/z): 432.2 (MH⁺), t_{R}=0.68 min; ¹H NMR (300 MHz, acetic acid-d⁴) δ ppm 1.00 (t, J=7.5 Hz, 3H), 1.23-1.41 (m, 4H), 1.75-1.93 (m, 2H), 2.32-2.50 (m, 1H), 2.62 (br. s., 3H), 3.06-3.25 (m, 2H), 7.07 (d, J=6.2 Hz, 1H), 7.29 (t, J=8.1 Hz, 1H), 7.45 (t, J=6.30 Hz, 1H), 7.70 (t, J=7.2 Hz, 1H), 8.41 (d, J=6.5 Hz, 1H).

### Example 7

### Preparation of (S)-methyl 1-(4-(2-tert-butyl-4-(2-chloro-3-(propylsulfonamido)phenyl)thiazol-5-yl)pyrimidin-2-ylamino)propan-2-ylcarbamate

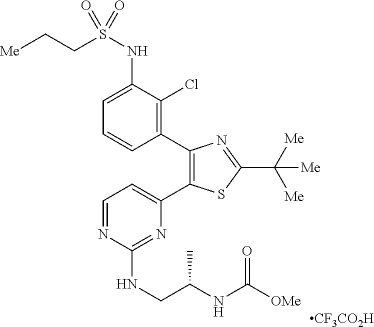 [SMILES: CCCS(=O)(=O)Nc1cccc(-c2nc(C(C)(C)C)sc2-c2ccnc(NC[C@H](C)NC(=O)OC)n2)c1Cl ; "·CF3CO2H"]

N-(3-(2-tert-butyl-5-(2-chloropyrimidin-4-yl)thiazol-4-yl)-2-chlorophenyl)propane-1-sulfonamide (XXVI, 30 mg, 0.062 mmol), DIEA (0.017 mL, 0.096 mmol), Na₂CO₃ (13 mg, 0.12 mmol), and (S)-tert-butyl 1-aminopropan-2-ylcarbamate (X, 11 mg, 0.062 mmol) were mixed in NMP (1 mL). The reaction mixture was heated at 90° C. for 3 days, cooled to room temperature and diluted with water. The water was adjusted to pH 5 with 1N HCl and extracted with EtOAc. The EtOAc layer was washed with brine, dried over magnesium sulfate, filtered and concentrated afford a crude (S)-tert-butyl 1-(4-(2-tert-butyl-4-(2-chloro-3-(propylsulfonamido)phenyl)thiazol-5-yl)pyrimidin-2-ylamino)propan-2-ylcarbamate (LCMS (m/z): 623.3 (MH⁺), t_{R}=1.05 min) as a yellow oil which was carried forward without further purification.

(S)-tert-butyl 1-(4-(2-tert-butyl-4-(2-chloro-3-(propylsulfonamido)phenyl)thiazol-5-yl)pyrimidin-2-ylamino)propan-2-ylcarbamate was dissolved in HCl (4M in dioxane, 2 mL) and allowed to stir at rt for 1 h. The reaction mixture was concentrated in vacuo to afford (S)—N-(3-(5-(2-(2-aminopropylamino)pyrimidin-4-yl)-2-tert-butylthiazol-4-yl)-2-chlorophenyl)propane-1-sulfonamide as a yellow solid (LCMS (m/z): 523.2 (MH⁺), t_{R}=0.77 min). This material was dissolved in THF (1.0 mL) and sat. aq. NaHCO₃ (1.0 mL), the mixture was cooled in an ice bath and methyl chloroformate (5 μL, 0.062 mmol) was added. The reaction was allowed to warm to room temperature and stirred overnight. The mixture was extracted with EtOAc, the aqueous layer was brought to a pH of 5 with 1M HCl, and extracted two more times with EtOAc. The organic extracts were combined, washed with water, brine, dried (MgSO₄), filtered and concentrated. The resulting residue was purified by reverse phase preparative HPLC. The fractions containing the pure product were combined and lyophilized to afford the desired (5)-methyl 1-(4-(2-tert-butyl-4-(2-chloro-3-(propylsulfonamido)phenyl)thiazol-5-yl)pyrimidin-2-ylamino)propan-2-ylcarbamate (6 mg) as the TFA salt:

LCMS (m/z): 581.2 (MH⁺), t_{R}=0.91 min; ¹H NMR (400 MHz, acetic acid-d⁴) δ ppm 1.00 (t, J=7.24Hz, 3H), 1.19 (d, J=6.26 Hz, 3H), 1.53 (s, 9H), 1.77-1.93 (m, 2H), 3.06-3.40 (m, 3H), 3.46-3.76 (m, 4H), 4.00 (br. s., 1H), 6.41 (br. s., 1H), 7.38 (d, J=7.83 Hz, 1H) 7.49 (t, J=7.83 Hz, 1H), 7.85 (d, J=8.22 Hz, 1H), 8.19 (d, J=6.26 Hz, 1H).

The following Table I (Examples 8-67) provides compounds made by the procedures described in examples 1-7 above, using the appropriate starting materials.

TABLE I

| | | LCMS: | HPLC: | |
|---|---|---|---|---|
| | | M + H, Rt | Rt | |
| Example | Structure | (min) | (min) | 1H NMR |
| 8 | 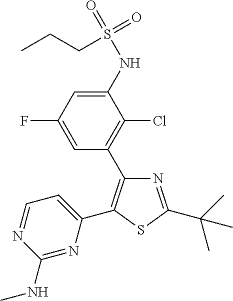 [SMILES: CCCS(=O)(=O)Nc1cc(F)cc(-c2nc(C(C)(C)C)sc2-c2ccnc(NC)n2)c1Cl] | 498.1, 0.95 | 3.63 | |
| 9 | 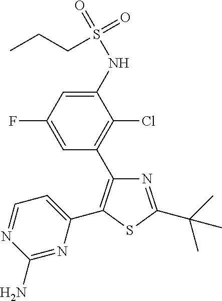 [SMILES: CCCS(=O)(=O)Nc1cc(F)cc(-c2nc(C(C)(C)C)sc2-c2ccnc(N)n2)c1Cl] | 484.0 0.89 | 3.38 | |
| 10 | 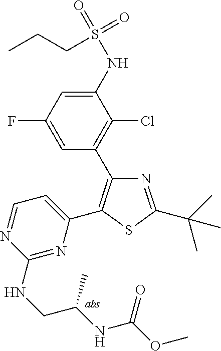 [SMILES: CCCS(=O)(=O)Nc1cc(F)cc(-c2nc(C(C)(C)C)sc2-c2ccnc(NC[C@H](C)NC(=O)OC)n2)c1Cl] | 599.2, 0.95 | 3.75 | |
| 11 | 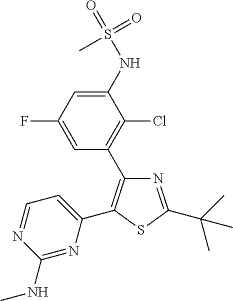 [SMILES: CNc1nccc(-c2sc(C(C)(C)C)nc2-c2cc(F)cc(NS(C)(=O)=O)c2Cl)n1] | 470.1, 0.82 | 3.29 | |
| 12 | 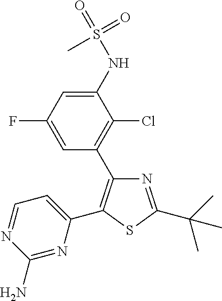 [SMILES: CC(C)(C)c1nc(-c2cc(F)cc(NS(C)(=O)=O)c2Cl)c(-c2ccnc(N)n2)s1] | 456.1, 0.77 | 3.06 | |
| 13 | 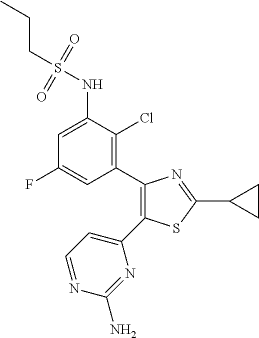 [SMILES: CCCS(=O)(=O)Nc1cc(F)cc(-c2nc(C3CC3)sc2-c2ccnc(N)n2)c1Cl] | 468.2, 0.76 | 3.03 | |
| 14 | 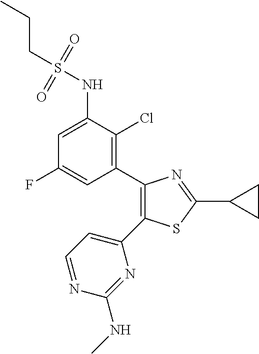 [SMILES: CCCS(=O)(=O)Nc1cc(F)cc(-c2nc(C3CC3)sc2-c2ccnc(NC)n2)c1Cl] | 482.2, 0.81 | 3.22 | ¹H NMR (300 MHz, acetic_acid) δ ppm 1.01 (t, J = 7.33 Hz, 3 H), 1.20 (m, 2 H), 1.30-1.41 (m, 2 H), 1.79-1.90 (m, 2 H), 2.49-2.64 (m, 1 H), 2.92 (s, 3 H), 3.12-3.33 (m, 2H), 6.39 (d, J = 6.15 Hz, 1 H), 7.15 (dd, J = 7.91, 2.64 Hz, 1 H), 7.65 (dd, J = 9.96, 2.64 Hz, 1 H), 8.21 (d, J = 6.15 Hz, 1 H) |
| 15 | 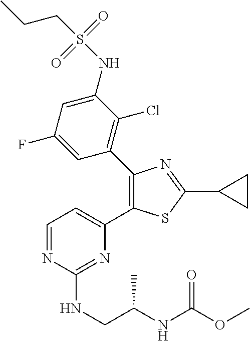 [SMILES: CCCS(=O)(=O)Nc1cc(F)cc(-c2nc(C3CC3)sc2-c2ccnc(NC[C@H](C)NC(=O)OC)n2)c1Cl] | 583.3, 0.85 | 3.36 | ¹H NMR (400 MHz, acetic_acid) δ ppm 1.01 (t, 3 H), 1.20 (d, J = 6.65 Hz, 5 H), 1.31-1.40 (m, 2 H), 1.88 (br.s., 2 H), 2.50-2.63 (m, 1 H), 3.17-3.24 (m, 2 H), 3.25 (m, 1H), 3.61 (s, 4 H), 4.01 (m, 1 H), 6.37 (br. s., 1 H), 7.16 (dd, J = 8.02, 2.54 Hz, 1 H), 7.66 (dd, J = 9.78, 2.74 Hz, 1 H), 8.20 (d, J = 6.26 Hz, 1 H) |
| 16 | 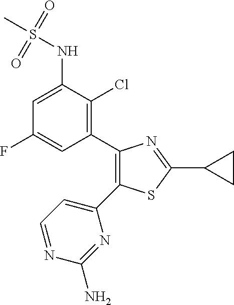 [SMILES: CS(=O)(=O)Nc1cc(F)cc(-c2nc(C3CC3)sc2-c2ccnc(N)n2)c1Cl] | 440.1, 0.64 | 2.66 | ¹H NMR (300 MHz, acetic_acid) δ ppm 1.15- 1.23 (m, 2 H), 1.31-1.41 (m, 2 H), 2.49-2.61 (m, 1 H), 3.13 (s, 3 H), 6.38 (d, J = 6.45 Hz, 1 H), 7.19 (dd, J = 8.20, 2.93 Hz, 1 H), 7.63 (dd, J = 9.82, 2.78 Hz, 1 H), 8.20 (d, J = 6.45 Hz, 1 H) |
| 17 | 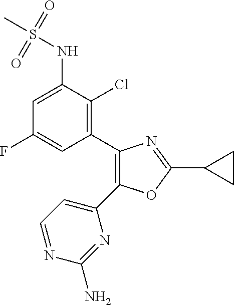 [SMILES: CS(=O)(=O)Nc1cc(F)cc(-c2nc(C3CC3)oc2-c2ccnc(N)n2)c1Cl] | 424.2, 0.59, | 2.42 | ¹H NMR (300 MHz, acetic_acid) δ ppm 1.26- 1.40 (m, 4 H), 2.34-2.46 (m, 1 H), 3.13 (s, 3 H), 6.88 (d, J = 6.15 Hz, 1 H), 7.22 (dd, J = 8.50, 2.93 Hz, 1 H), 7.57 (dd, J = 9.52, 2.78 Hz, 1 H), 8.36 (d, J = 6.15 Hz, 1 H) |
| 18 | 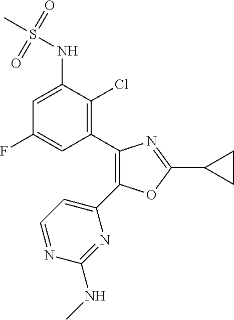 [SMILES: CNc1nccc(-c2oc(C3CC3)nc2-c2cc(F)cc(NS(C)(=O)=O)c2Cl)n1] | 438.2, 0.64 | 2.63 | ¹H NMR (300 MHz, acetic_acid) δ ppm 1.28- 1.38 (m, 4 H), 2.34-2.46 (m, 1 H), 2.55 (br. s., 3 H), 3.13 (s, 3 H), 7.06 (d, J = 5.86 Hz, 1 H), 7.20 (dd, J = 8.20, 2.93 Hz, 1 H), 7.56 (dd, J = 9.67, 2.93 Hz, 1 H), 8.42 (d, J = 6.15 Hz, 1 H) |
| 19 | 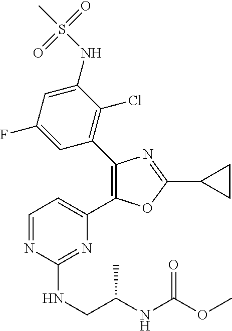 [SMILES: COC(=O)N[C@@H](C)CNc1nccc(-c2oc(C3CC3)nc2-c2cc(F)cc(NS(C)(=O)=O)c2Cl)n1] | 539.3, 0.67 | 2.78 | ¹H NMR (300 MHz, acetic_acid) δ ppm 0.98 (d, J = 5.86 Hz, 3 H), 1.27-1.37 (m, 4 H), 2.33- 2.47 (m, 1 H), 2.84-3.09 (m, 2 H), 3.14 (s, 3 H), 3.60 (s, 4H), 7.08 (d, J = 5.86 Hz, 1 H), 7.23 (dd, J = 8.20, 2.93 Hz, 1 H), 7.58 (dd, J = 9.82, 2.78 Hz, 1 H), 8.40 (d, J = 6.15 Hz, 1 H) |
| 20 | 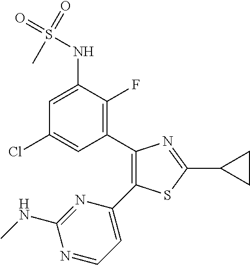 [SMILES: CNc1nccc(-c2sc(C3CC3)nc2-c2cc(Cl)cc(NS(C)(=O)=O)c2F)n1] | 454.2/0.75 | 2.99 | 1H NMR (300 MHz, acetic_acid) δ ppm 1.16- 1.28 (m, 2 H), 1.28-1.42 (m, 2 H), 2.47-2.62 (m, 1 H), 2.94 (s, 3 H), 3.11 (s, 3 H), 6.58- 6.71 (m, 1 H), 7.41-7.52 (m, 1 H), 7.68-7.80 (m, 1 H), 8.27 (d, J = 6.45 Hz, 1 H) |
| 21 | 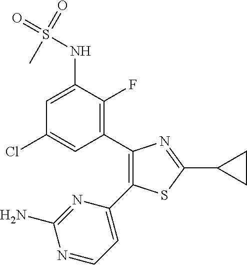 [SMILES: CS(=O)(=O)Nc1cc(Cl)cc(-c2nc(C3CC3)sc2-c2ccnc(N)n2)c1F] | 440.1/0.69 | 2.77 | 1H NMR (300 MHz, acetic_acid) δ ppm 1.08- 1.23 (m, 2 H), 1.23-1.43 (m, 2 H), 2.43-2.59 (m, 1 H), 3.09 (s, 3 H), 6.59 (d, J = 5.86 Hz, 1 H), 7.41-7.53 (m, 1 H), 7.66-7.79 (m, 1 H), 8.22 (d, J = 5.86 Hz, 1 H) |
| 22 | 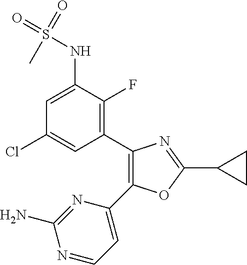 [SMILES: CS(=O)(=O)Nc1cc(Cl)cc(-c2nc(C3CC3)oc2-c2ccnc(N)n2)c1F] | 424.0/0.66 | 2.56 | 1H NMR (300 MHz, acetic_acid) δ ppm 1.21- 1.41 (m, 4 H), 2.30-2.44 (m, 1 H), 3.13 (s, 3 H), 6.95-7.06 (m, 1 H), 7.46-7.56 (m, 1 H), 7.66 (dd, J = 6.45, 2.64 Hz, 1 H), 8.38 (d, J = 6.15 Hz, 1 H) |
| 23 | 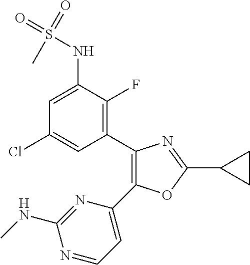 [SMILES: CNc1nccc(-c2oc(C3CC3)nc2-c2cc(Cl)cc(NS(C)(=O)=O)c2F)n1] | 438.1/0.68 | 2.70 | 1H NMR (300 MHz, acetic_acid) δ ppm 1.33 (br. s., 4 H), 2.31-2.48 (m, 1 H), 2.70 (br. s., 3 H), 3.13 (s, 3 H), 7.13 (d, J = 6.45 Hz, 1 H), 7.45-7.57 (m, 1 H), 7.66-7.80 (m, 1 H), 8.46 (d, J = 6.45 Hz, 1 H) |
| 24 | 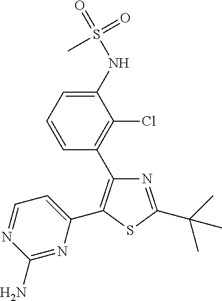 [SMILES: CC(C)(C)c1nc(-c2cccc(NS(C)(=O)=O)c2Cl)c(-c2ccnc(N)n2)s1] | 437.9, 0.69 | 2.83 | 1H NMR (400 MHz, DMSO-d 6): δ ppm 1.43 (s, 2 H), 3.07 (s, 3 H), 4.27 (br. s., 9 H), 5.91 (d, J = 5.48 Hz, 1 H), 6.67-7.22 (m, 1 H), 7.37 (dd, J = 7.63, 1.37 Hz, 1 H), 7.49 (t, J = 7.83 Hz, 1 H), 7.63 (dd, J = 8.22, 1.17 Hz, 1 H), 8.04 (d, J = 5.48 Hz, 1 H), 9.56 (s, 1 H) |
| 25 | 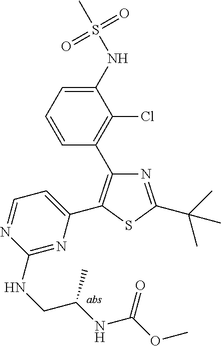 [SMILES: COC(=O)N[C@@H](C)CNc1nccc(-c2sc(C(C)(C)C)nc2-c2cccc(NS(C)(=O)=O)c2Cl)n1] | 553.0, 0.78 | 3.52 | 1H NMR (400 MHz, acetic_acid) δ ppm 1.19 (d, J = 6.65 Hz, 3 H), 1.53 (s, 9 H), 3.10 (s, 3 H), 3.27 (br. s., 1 H), 3.47-3.76 (m, 4 H), 3.84- 4.08 (m, 1H), 6.42 (d, J = 5.09 Hz, 1 H), 7.40 (d, J = 6.65 Hz, 1 H), 7.51 (t, J = 7.83 Hz, 1 H), 7.82 (d, J = 7.43 Hz, 1 H), 8.19 (d, J = 6.26 Hz, 1 H) |
| 26 | 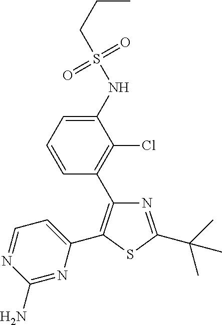 [SMILES: CCCS(=O)(=O)Nc1cccc(-c2nc(C(C)(C)C)sc2-c2ccnc(N)n2)c1Cl] | 465.9, 0.79 | 3.50 | 1H NMR (400 MHz, DMSO-d 6) δ ppm 0.87 (t, J = 7.43 Hz, 3 H), 1.36 (s, 9 H), 1.58-1.77 (m, 2 H), 2.98-3.16 (m, 2 H), 5.80 (d, J = 5.48 Hz, 1 H), 6.82 (br. s., 1 H), 7.29 (dd, J = 7.63, 1.76 Hz, 1 H), 7.41 (t, J = 8.02 Hz, 1 H), 7.56 (dd, J = 8.22, 1.57 Hz, 1 H), 7.96 (d, J = 5.09 Hz, 1 H) 9.49 (s, 1 H) |
| 27 | 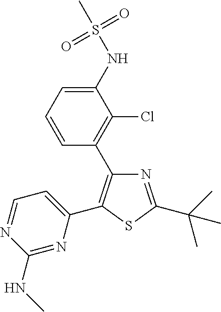 [SMILES: CNc1nccc(-c2sc(C(C)(C)C)nc2-c2cccc(NS(C)(=O)=O)c2Cl)n1] | 452.2, 0.76 | 3.38 | 1H NMR (300 MHz, acetic_acid) δ ppm 1.53 (s, 9 H), 2.92 (s, 3 H), 3.09 (s, 3 H), 6.40 (d, 1 H), 7.40 (dd, J = 7.62, 1.47 Hz, 1 H), 7.50 (t, J = 7.91 Hz, 1H), 7.81 (dd, J = 8.20, 1.47 Hz, 1 H), 8.19 (d, J = 6.45 Hz, 1 H) |
| 28 | 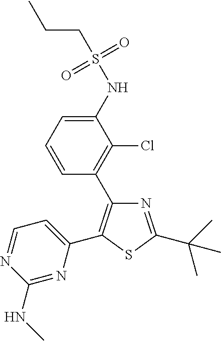 [SMILES: CCCS(=O)(=O)Nc1cccc(-c2nc(C(C)(C)C)sc2-c2ccnc(NC)n2)c1Cl] | 480.2, 0.86 | 3.98 | 1H NMR (300 MHz, acetic_acid) δ ppm 1.00 (t, J = 7.47 Hz, 3 H), 1.53 (s, 9 H), 1.78-1.93 (m, 2 H), 2.93 (s, 3 H), 3.10-3.23 (m, 2 H), 6.42 (d, J = 6.45 Hz, 1 H), 7.35-7.39 (m, 1 H), 7.49 (t, J = 7.91 Hz, 1 H), 7.85 (dd, J = 8.20, 1.76 Hz, 1 H), 8.21 (d, J = 6.45 Hz, 1 H) |
| 29 | 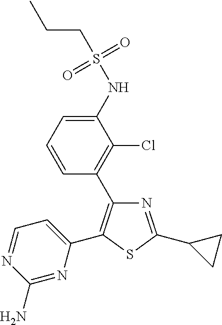 [SMILES: CCCS(=O)(=O)Nc1cccc(-c2nc(C3CC3)sc2-c2ccnc(N)n2)c1Cl] | 450.2, 0.69 | 2.69 | 1H NMR (400 MHz, acetic_acid) δ ppm 1.00 (t, J = 7.43 Hz, 3 H), 1.18 (dd, J = 4.30, 2.74 Hz, 2 H), 1.30-1.42 (m, 2 H), 1.72-1.89 (m, 2 H), 2.48-2.63 (m, 1 H), 3.11-3.25 (m, 2 H), 6.25 (d, J = 6.26 Hz, 1 H), 7.36 (d, J = 7.43 Hz, 1 H), 7.49 (t, J = 7.83 Hz, 1 H), 7.86 (d, J = 7.83 Hz, 1 H), 8.13 (d, J = 6.65 Hz, 1 H) |
| 30 | 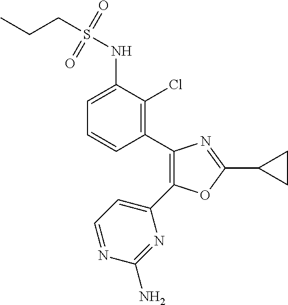 [SMILES: CCCS(=O)(=O)Nc1cccc(-c2nc(C3CC3)oc2-c2ccnc(N)n2)c1Cl] | 434.2, 0.65 | 2.48 | 1H NMR (300 MHz, acetic_acid) δ ppm 1.01 (t, J = 7.33 Hz, 3 H), 1.21-1.43 (m, 4 H), 1.76- 1.93 (m, 2 H), 2.33-2.49 (m, 1 H), 3.09-3.30 (m, 2 H), 6.76 (d, J = 6.15 Hz, 1 H), 7.33-7.53 (m, 2 H), 7.80 (dd, J = 7.62, 2.05 Hz, 1 H), 8.30 (d, J = 6.15 Hz, 1 H) |
| 31 | 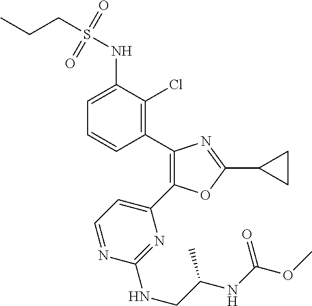 [SMILES: CCCS(=O)(=O)Nc1cccc(-c2nc(C3CC3)oc2-c2ccnc(NC[C@H](C)NC(=O)OC)n2)c1Cl] | 549.3, 0.72 | 3.15 | 1H NMR (300 MHz, acetic_acid) δ ppm 0.93 (d, J = 4.98 Hz, 3 H), 1.01 (t, J = 7.47 Hz, 3 H), 1.24-1.40 (m, 4 H), 1.77-1.93 (m, 2 H), 2.34- 2.51 (m, 1 H), 2.74-3.12 (m, 2 H), 3.12- 3.27 (m, 2 H), 3.60 (s, 4 H), 7.07 (d, J = 4.69 Hz, 1 H), 7.35-7.54 (m, 2 H), 7.81 (dd, J = 7.91, 1.47 Hz, 1 H), 8.39 (d, J = 6.15 Hz, 1 H) |
| 32 | 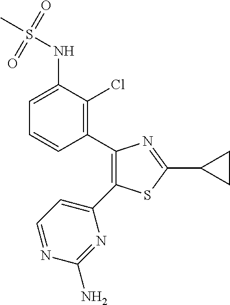 [SMILES: CS(=O)(=O)Nc1cccc(-c2nc(C3CC3)sc2-c2ccnc(N)n2)c1Cl] | 422.1, 0.59 | 2.17 | 1H NMR (300 MHz, acetic_acid) δ ppm 1.13- 1.26 (m, 2 H), 1.29-1.44 (m, 2 H), 2.48-2.65 (m, 1 H), 3.09 (s, 3 H), 6.30 (d, J = 6.45 Hz, 1 H), 7.35-7.44 (m, 1 H), 7.51 (t, J = 7.91 Hz, 1 H), 7.83 (dd, J = 8.20, 1.47 Hz, 1 H), 8.16 (d, J = 6.45 Hz, 1 H) |
| 33 | 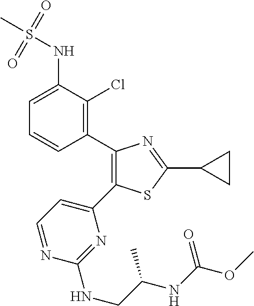 | 537.3, 0.68 | 2.01 | 1H NMR (300 MHz, acetic_acid) δ ppm 1.20 (d, J = 6.74 Hz, 5 H), 1.28-1.44 (m, 2 H), 2.50- 2.66 (m, 1 H), 3.09 (s, 3 H), 3.20-3.47 (m, 1 H), 3.48 3.79 (m, 4 H), 3.87-4.14 (m, 1 H), 6.34 (d, J = 6.15 Hz, 1 H), 7.33-7.43 (m, 1 H), 7.51 (t, J = 7.91 Hz, 1 H), 7.83 (dd, J = 8.06, 1.32 Hz, 1 H), 8.17 (d, J = 6.45 Hz, 1 H) |
| 34 | 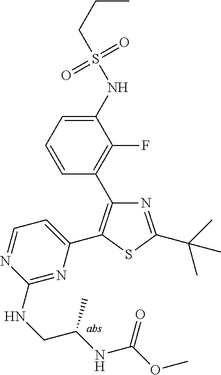 [SMILES: CCCS(=O)(=O)Nc1cccc(-c2nc(C(C)(C)C)sc2-c2ccnc(NC[C@H](C)NC(=O)OC)n2)c1F] | 565.3, 0.88 | 4.15 | 1H NMR (400 MHz, Acetic-d3-acid-d) δ ppm: 0.99 (t, J = 7.4 Hz, 3H), 1.16 (d, J = 6.7 Hz, 3H), 1.53 (s, 9H), 1.76-1.86 (m, 2H), 3.14 (m, 2H), 3.60 (s, 3H), 3.66 (br. s., 2H), 3.95 (br. s., 1H), 6.61 (d, J = 8.0 Hz, 1 H), 7.33 (t, J = 7.8 Hz, 1H), 7.44 (m, 1 H), 7.74 (m, 1H), 8.23 (d, J = 8.0 Hz, 1H) |
| 35 | 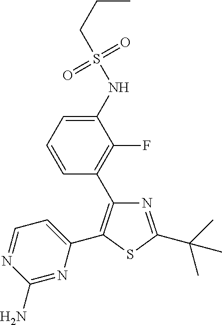 [SMILES: CCCS(=O)(=O)Nc1cccc(-c2nc(C(C)(C)C)sc2-c2ccnc(N)n2)c1F] | 450.0, 0.80 | 3.51 | 1H NMR (400 MHz, Acetic-d3-acid-d) δ ppm: 0.99 (t, J = 7.3 Hz, 4H,), 1.52 (s, 9H), 1.73- 1.92 (m, 2H), 3.05-3.22 (m, 2H), 6.56 (d, J = 6.6 Hz, 1 H), 7.33 (t, J = 8.0 Hz, 1H), 7.45 (m, 1H), 7.74 (m, 1H), 8.21 (d, J = 6.6 Hz, 1H) |
| 36 | 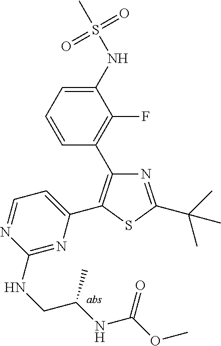 [SMILES: COC(=O)N[C@@H](C)CNc1nccc(-c2sc(C(C)(C)C)nc2-c2cccc(NS(C)(=O)=O)c2F)n1] | 537.3, 0.80 | 3.59 | |
| 37 | 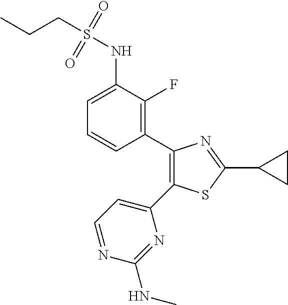 [SMILES: CCCS(=O)(=O)Nc1cccc(-c2nc(C3CC3)sc2-c2ccnc(NC)n2)c1F] | 448.2, 0.74 | 3.16 | 1H NMR (300 MHz, Acetic-d3-acid-d): δ ppm 0.99 (t, 3 H), 1.14-1.26 (m, 2 H), 1.28-1.41 (m, 2 H), 1.73-1.93 (m, 2 H), 2.56 (m, 1 H), 2.95 (s, 3 H), 3.15 (m, 2 H), 6.52 (d, J = 6.45 Hz, 1 H), 7.24-7.49 (m, 2 H), 7.75 (td, J = 7.77, 1.47 Hz, 1 H), 8.21 (d, J = 6.45 Hz, 1 H) |
| 38 | 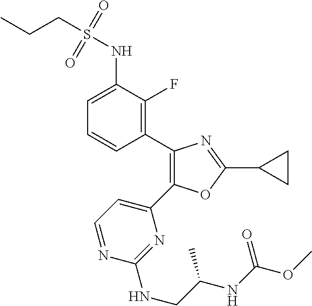 [SMILES: CCCS(=O)(=O)Nc1cccc(-c2nc(C3CC3)oc2-c2ccnc(NC[C@H](C)NC(=O)OC)n2)c1F] | 533.3, 0.71 | 3.09 | 1H NMR (400 MHz, acetic_acid) δ ppm 0.89 (br. s., 3 H), 1.00 (t, J = 7.63 Hz, 3 H), 1.22- 1.41 (m, 4 H), 1.85 (m, 2 H), 2.41 (m, 1 H), 2.95 (m, 1 H), 3.16 (m, 3 H), 3.58 (s, 4 H), 7.11 (br. s., 1 H), 7.32 (t, J = 8.02 Hz, 1 H), 7.48 (t, J = 6.85 Hz, 1 H), 7.73 (t, J = 7.43 Hz, 1 H), 8.43 (d, J = 6.26 Hz, 1 H) |
| 39 | 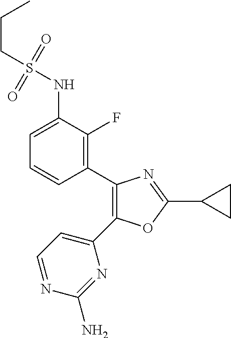 [SMILES: CCCS(=O)(=O)Nc1cccc(-c2nc(C3CC3)oc2-c2ccnc(N)n2)c1F] | 418.2, 0.63 | 2.48 | 1H NMR (300 MHz, acetic_acid) δ ppm 1.01 (m, 3 H), 1.21-1.41 (m, 4 H), 1.84 (m, 2 H), 2.40 (m, 1 H), 3.15 (m, 2 H), 6.99 (d, J = 6.15 Hz, 1 H), 7.29 (m, 1 H), 7.49 (m, 1 H), 7.66 (m, 1 H), 8.38 (d, J = 6.45 Hz, 1 H) |
| 40 | 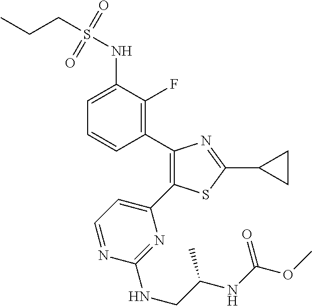 [SMILES: CCCS(=O)(=O)Nc1cccc(-c2nc(C3CC3)sc2-c2ccnc(NC[C@H](C)NC(=O)OC)n2)c1F] | 549.4, 0.77 | 3.47 | 1H NMR (300 MHz, acetic_acid) δ ppm 1.00 (t, J = 7.47 Hz, 3 H), 1.18 (d, 3 H, J = 6.0 Hz), 1.11- 1.27 (m, 2 H), 1.27-1.43 (m, 2 H), 1.75- 1.92 (m, 2 H), 2.56 (m,1 H), 3.16 (m, 2 H), 3.29 (m, 1 H), 3.60 (s, 3 H), 3.51-3.55 (m, 1H), 3.99 (m, 1 H), 6.56 (d, J = 6.45 Hz, 1 H), 7.33 (m, 1 H), 7.42 (m, 1 H), 7.75 (td, J = 7.84, 1.90 Hz, 1 H), 8.22 (d, J = 6.45 Hz, 1 H) |
| 41 | 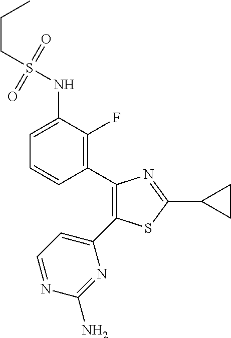 [SMILES: CCCS(=O)(=O)Nc1cccc(-c2nc(C3CC3)sc2-c2ccnc(N)n2)c1F] | 434.2, 0.68 | 2.8 | 1H NMR (300 MHz, acetic_acid) δ ppm 0.99 (t, J = 7.50, 3 H), 1.11-1.25 (m, 2 H), 1.26-1.40 (m, 2 H), 1.73-1.90 (m, 2 H), 2.46-2.61 (m, 1 H), 3.08-3.22 (m, 2 H), 6.51 (d, J = 6.45 Hz, 1 H), 7.32 (m, 1 H), 7.41 (m, 1 H), 7.75 (m, 1 H), 8.19 (d, J = 6.45 Hz, 1 H) |
| 42 | 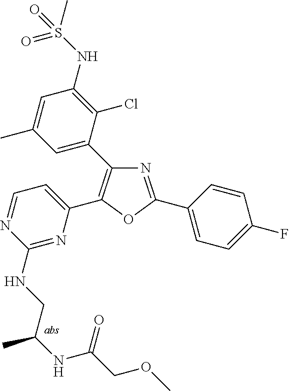 [SMILES: COCC(=O)N[C@@H](C)CNc1nccc(-c2oc(-c3ccc(F)cc3)nc2-c2cc(C)cc(NS(C)(=O)=O)c2Cl)n1] | 603.1, 0.81 | 0.86 (2 min UPLC) | |
| 43 | 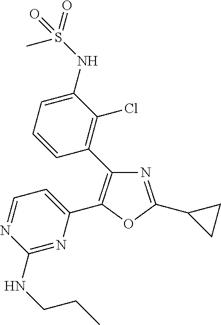 [SMILES: CCCNc1nccc(-c2oc(C3CC3)nc2-c2cccc(NS(C)(=O)=O)c2Cl)n1] | 448.1, 0.69 | 2.75 | |
| 44 | 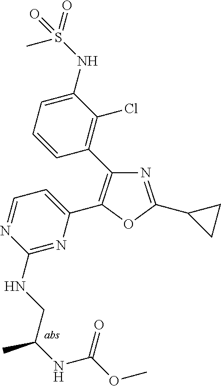 [SMILES: COC(=O)N[C@@H](C)CNc1nccc(-c2oc(C3CC3)nc2-c2cccc(NS(C)(=O)=O)c2Cl)n1] | 521.2, 0.64 | 2.50 | |
| 45 | 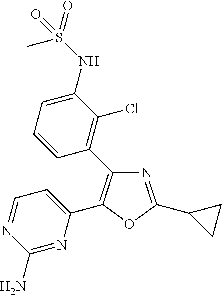 [SMILES: CS(=O)(=O)Nc1cccc(-c2nc(C3CC3)oc2-c2ccnc(N)n2)c1Cl] | 406.0, 0.56 | 1.87 | |
| 46 | 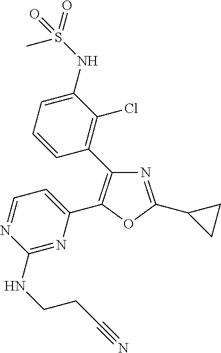 [SMILES: CS(=O)(=O)Nc1cccc(-c2nc(C3CC3)oc2-c2ccnc(NCCC#N)n2)c1Cl] | 459.1, 0.64 | 0.63 (2 min UPLC) | |
| 47 | 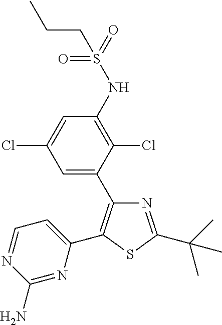 [SMILES: CCCS(=O)(=O)Nc1cc(Cl)cc(-c2nc(C(C)(C)C)sc2-c2ccnc(N)n2)c1Cl] | 500.0/502.0, 0.92 | 3.59 | 1H NMR (400 MHz, DMSO-d6) δ ppm 0.93 (t, J = 7.43 Hz, 3 H), 1.41 (s, 9 H), 1.64-1.79 (m, 2 H), 3.09-3.24 (m, 2 H), 5.94 (s, 1 H), 7.48 (d, J = 1.96 Hz, 1 H), 7.64 (d, J = 2.35 Hz, 1 H), 8.06 (d, J = 5.09 Hz, 1 H), 9.76 (s, 1H) |
| 48 | 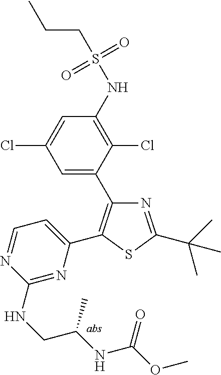 [SMILES: CCCS(=O)(=O)Nc1cc(Cl)cc(-c2nc(C(C)(C)C)sc2-c2ccnc(NC[C@H](C)NC(=O)OC)n2)c1Cl] | 615.2/617.2, 1.00 | 1.03 (2 min UPLC) | 1H NMR (400 MHz, acetic_acid) δ ppm 1.01 (t, J = 7.43 Hz, 3 H), 1.19 (d, J = 6.65 Hz, 3 H), 1.53 (s, 9 H), 1.80-1.93 (m, 2 H), 3.14-3.30 (m, 3 H), 3.49-3.65 (m, 3 H), 3.69 (br. s., 1 H), 3.99 (m, 1 H), 6.55 (d, J = 5.09 Hz, 1 H), 7.42 (d, J = 2.35 Hz, 1 H), 7.87 (d, J = 1.96 Hz, 1 H), 8.27 (d, J = 6.65 Hz, 1 H) |
| 49 | 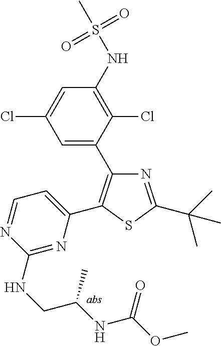 [SMILES: COC(=O)N[C@@H](C)CNc1nccc(-c2sc(C(C)(C)C)nc2-c2cc(Cl)cc(NS(C)(=O)=O)c2Cl)n1] | 587.0/589.0, 0.90 | 3.55 | 1H NMR (400 MHz, acetic_acid) δ ppm 1.19 (d, J = 6.65 Hz, 3 H), 1.53 (s, 9 H), 3.14 (s, 3 H), 3.44-3.75 (m, 5 H), 3.98 (m, 1 H), 6.54 (d, J = 4.70 Hz, 1 H), 7.44 (d, J = 2.35 Hz, 1 H), 7.83 (d, J = 2.35 Hz, 1 H), 8.25 (d, J = 6.26 Hz, 1 H) |
| 50 | 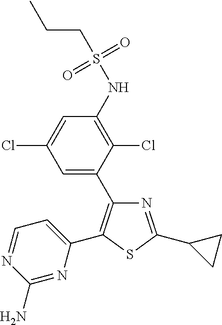 [SMILES: CCCS(=O)(=O)Nc1cc(Cl)cc(-c2nc(C3CC3)sc2-c2ccnc(N)n2)c1Cl] | 484.2/486.2, 0.79 | 3.57 | 1H NMR (400 MHz, acetic_acid) δ ppm 1.01 (t, J = 7.43 Hz, 3 H), 1.19 (m, 2 H), 1.35 (m, 2 H), 1.79-1.92 (m, 2 H), 2.48-2.59 (m, 1 H), 3.14- 3.26 (m, 2 H), 6.39 (d, J = 6.65 Hz, 1 H), 7.40 (d, J = 2.35 Hz, 1 H), 7.88 (d, J = 2.35 Hz, 1 H), 8.22 (d, J = 6.65 Hz, 1 H) |
| 51 | 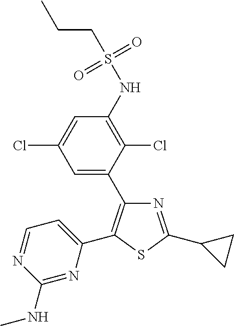 [SMILES: CCCS(=O)(=O)Nc1cc(Cl)cc(-c2nc(C3CC3)sc2-c2ccnc(NC)n2)c1Cl] | 498.2/500.2, 0.85 | 3.97 | 1H NMR (400 MHz, acetic_acid) δ ppm 1.01 (t, J = 7.43 Hz, 3 H), 1.18-1.26 (m, 2 H), 1.36 (dd, J = 8.02, 2.54 Hz, 2 H), 1.76-1.91 (m, 2 H), 2.50-2.61 (m, 1 H), 2.92 (s, 3 H), 3.15-3.26 (m, 2 H), 6.45 (d, J = 6.65 Hz, 1 H), 7.39 (d, J = 2.74 Hz, 1 H), 7.86 (d, J = 2.35 Hz, 1 H), 8.25 (d, J = 6.65 Hz, 1 H) |
| 52 | 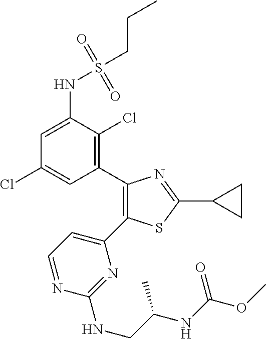 [SMILES: CCCS(=O)(=O)Nc1cc(Cl)cc(-c2nc(C3CC3)sc2-c2ccnc(NC[C@H](C)NC(=O)OC)n2)c1Cl] | 599.4/601.4, 0.87 | 4.20 | |
| 53 | 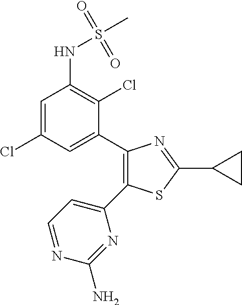 [SMILES: CS(=O)(=O)Nc1cc(Cl)cc(-c2nc(C3CC3)sc2-c2ccnc(N)n2)c1Cl] | 456.1/458.1, 0.69 | 2.91 | |
| 54 | 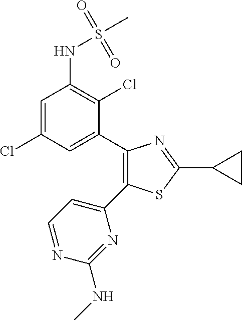 [SMILES: CNc1nccc(-c2sc(C3CC3)nc2-c2cc(Cl)cc(NS(C)(=O)=O)c2Cl)n1] | 470.2/472.2, 0.74 | 3.26 | |
| 55 | 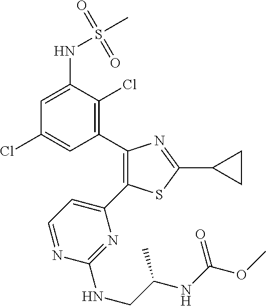 [SMILES: COC(=O)N[C@@H](C)CNc1nccc(-c2sc(C3CC3)nc2-c2cc(Cl)cc(NS(C)(=O)=O)c2Cl)n1] | 571.2/573.2, 0.79 | 3.23 | |
| 56 | 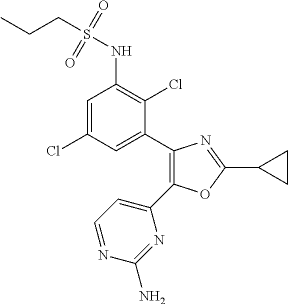 [SMILES: CCCS(=O)(=O)Nc1cc(Cl)cc(-c2nc(C3CC3)oc2-c2ccnc(N)n2)c1Cl] | 468/470, 0.75 | 3.30 | 1H NMR (300 MHz, acetic_acid) δ ppm 1.01 (t, J = 7.47 Hz, 3 H), 1.18-1.43 (m, 4 H), 1.78- 1.94 (m, 2H), 2.37 (m, 1 H), 3.09-3.30 (m, 2 H), 6.87 (d, J = 5.86 Hz, 1 H), 7.42 (d, J = 2.05 Hz, 1 H), 7.81 (d, J = 2.34 Hz, 1 H), 8.35 (d, J = 5.86 Hz, 1 H) |
| 57 | 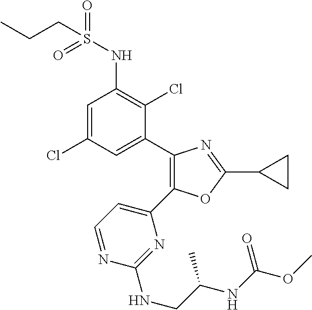 [SMILES: CCCS(=O)(=O)Nc1cc(Cl)cc(-c2nc(C3CC3)oc2-c2ccnc(NC[C@H](C)NC(=O)OC)n2)c1Cl] | 583.4/585.4, 0.82 | 3.24 | 1H NMR (300 MHz, acetic_acid) δ ppm 0.88 (t, J = 7.33 Hz, 3 H), 1.17 (bs, 7 H), 1.72-1.79 (m, 2H), 2.24 (m, 1 H) 2.77 (br. s., 1H), 2.92 (br. s., 1 H), 3.01-3.15 (m, 2 H), 3.46 (s, 4 H), 6.93 (br. s., 1 H), 7.30 (d, J = 2.34 Hz, 1 H), 7.68 (d, J = 2.34 Hz, 1 H), 8.27 (br. s., 1 H) |
| 58 | 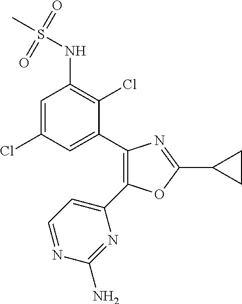 [SMILES: CS(=O)(=O)Nc1cc(Cl)cc(-c2nc(C3CC3)oc2-c2ccnc(N)n2)c1Cl] | 440/442, 0.66 | 2.61 | 1H NMR (300 MHz, acetic_acid) δ ppm 1.23- 1.41 (m, 4 H), 2.33-2.46 (m, 1 H), 3.13 (s, 3 H), 6.90 (d, J = 6.15 Hz, 1 H), 7.45 (d, J = 2.34 Hz, 1 H), 7.78 (d, J = 2.34 Hz, 1 H), 8.36 (d, J = 6.15 Hz, 1 H) |
| 59 | 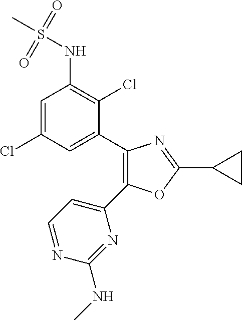 [SMILES: CNc1nccc(-c2oc(C3CC3)nc2-c2cc(Cl)cc(NS(C)(=O)=O)c2Cl)n1] | 454/456, 0.69 | 2.87 | 1H NMR (300 MHz, acetic_acid) δ ppm 1.23- 1.40 (m, 4 H), 2.34-2.46 (m, 1 H), 2.55 (br. s., 3 H), 3.13 (s, 3H), 7.05 (d, J = 5.86 Hz, 1 H), 7.44 (d, J = 2.34 Hz, 1 H), 7.77 (d, J = 2.64 Hz, 1 H), 8.41 (d, J = 6.15 Hz, 1 H), |
| 60 | 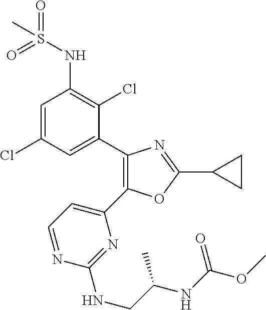 | 555.3/557.3, 0.73 | 3.15 | 1H NMR (300 MHz, acetic_acid) δ ppm 0.97 (d, J = 5.86 Hz, 3 H), 1.31 (m, 4 H), 2.34-2.46 (m, 1H), 2.91 (br. s., 1 H), 3.01-3.11 (m, 1 H), 3.14 (s, 3 H), 3.60 (s, 4 H), 7.08 (br. s., 1 H), 7.47 (d, J = 2.34 Hz, 1 H), 7.79 (d, J = 2.34 Hz, 1 H) , 8.41 (d, J = 6.15 Hz, 1 H) |
| 61 | 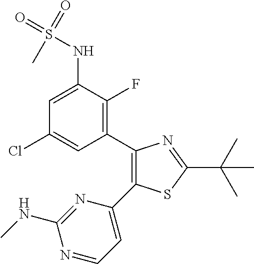 [SMILES: CNc1nccc(-c2sc(C(C)(C)C)nc2-c2cc(Cl)cc(NS(C)(=O)=O)c2F)n1] | 470.2/0.89 | 4.05 | 1H NMR (400 MHz, acetic_acid) δ ppm 1.53 (s, 9 H), 2.93 (s, 3 H), 3.11 (s, 3 H), 6.68-6.76 (m, 1 H), 7.43-7.52 (m, 1 H), 7.69-7.78 (m, 1 H), 8.25-8.35 (m, 1 H) |
| 62 | 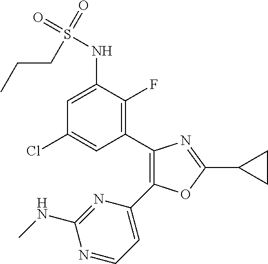 [SMILES: CCCS(=O)(=O)Nc1cc(Cl)cc(-c2nc(C3CC3)oc2-c2ccnc(NC)n2)c1F] | 446.1/0.78 | 3.47 | 1H NMR (400 MHz, acetic_acid) δ ppm 0.88 (t, J = 7.43 Hz, 3 H), 1.09-1.24 (m, 4 H), 1.63- 1.80 (m, 2 H), 2.19-2.37 (m, 1 H), 2.55 (br. s., 3 H), 3.07 (br. s., 2 H), 6.87-7.02 (m, 1 H) 7.34 (d, J = 3.13 Hz, 1 H), 7.54-7.66 (m, 1 H), 8.24-8.37 (m, 1 H). |
| 63 | 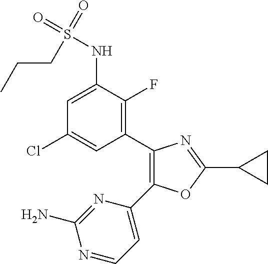 [SMILES: CCCS(=O)(=O)Nc1cc(Cl)cc(-c2nc(C3CC3)oc2-c2ccnc(N)n2)c1F] | 452.1/0.74 | 3.21 | 1H NMR (300 MHz, acetic_acid) δ ppm 0.87 (t, J = 7.47 Hz, 3 H), 1.07-1.34 (m, 4 H), 1.60- 1.82 (m, 2 H), 2.15-2.32 (m, 1 H), 2.96-3.16 (m, 2 H), 6.83 (d, J = 5.86 Hz, 1 H), 7.34 (dd, J = 5.42, 2.49 Hz, 1 H), 7.49-7.64 (m, 1 H), 8.23 (d, J = 6.15 Hz, 1 H) |
| 64 | 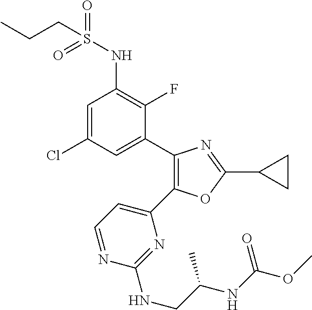 [SMILES: CCCS(=O)(=O)Nc1cc(Cl)cc(-c2nc(C3CC3)oc2-c2ccnc(NC[C@H](C)NC(=O)OC)n2)c1F] | 567.3/0.80 | 3.70 | 1H NMR (300 MHz, acetic_acid) δ ppm 0.71- 0.98 (m, 6 H), 1.08-1.29 (m, 4 H), 1.72 (m, 2 H), 2.20-2.34 (m, 1 H), 2.79-3.00 (m, 1 H), 3.00-3.18 (m, 3 H), 3.45 (s, 4 H), 6.99 (br. s., 1 H), 7.34 (dd, J = 5.27, 2.34 Hz, 1 H), 7.60 (dd, J = 6.59, 2.20 Hz, 1 H), 8.31 (d, J = 6.15 Hz, 1 H). |
| 65 | 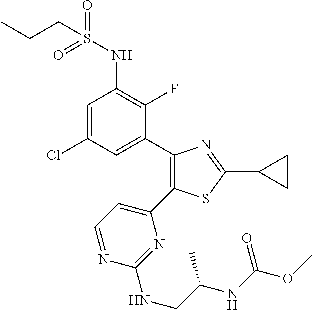 [SMILES: CCCS(=O)(=O)Nc1cc(Cl)cc(-c2nc(C3CC3)sc2-c2ccnc(NC[C@H](C)NC(=O)OC)n2)c1F] | 583.3/0.87 | 4.15 | 1H NMR (300 MHz, acetic_acid) δ ppm 0.86 (t, 3 H), 0.97-1.13 (m, 5 H), 1.15-1.30 (m, 2 H), 1.60-1.77 (m, 2 H), 2.41 (m, 1 H), 2.97- 3.26 (m, 3 H), 3.47 (s, 4 H), 3.71-3.93 (m, 1 H), 6.42-6.60 (m, 1 H),7.25-7.37 (m, 1 H), 7.58-7.69 (m, 1 H), 8.07-8.23 (m, 1 H) |
| 66 | 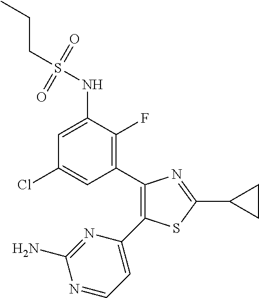 [SMILES: CCCS(=O)(=O)Nc1cc(Cl)cc(-c2nc(C3CC3)sc2-c2ccnc(N)n2)c1F] | 467.9/0.78 | 3.52 | 1H NMR (300 MHz, acetic_acid) δ ppm 0.99 (t, J = 7.33 Hz, 3 H), 1.25-1.42 (m, 4 H), 1.82 (d, J = 7.62 Hz, 2 H), 2.52 (m, 1 H), 3.13-3.26 (m, 2 H), 6.55-6.67 (m, 1 H) 7.40-7.49 (m, 1 H) 7.69-7.83 (m, 1 H) 8.18-8.31 (m, 1 H). |

Compounds listed in Table II can be made by the procedures described above, using the appropriate starting materials.

TABLE II

| Example | Structure | Compound Name |
|---|---|---|
| P1 | 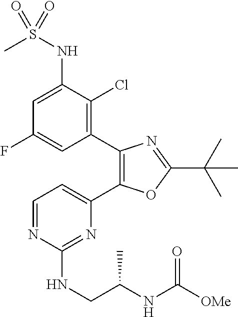 [SMILES: COC(=O)N[C@@H](C)CNc1nccc(-c2oc(C(C)(C)C)nc2-c2cc(F)cc(NS(C)(=O)=O)c2Cl)n1] | (S)-methyl 1-(4-(2-tert-butyl-4-(2- chloro-5-fluoro-3- (methylsulfonamido)phenyl)oxazol- 5-yl)pyrimidin-2-ylamino)propan-2- ylcarbamate |
| P2 | 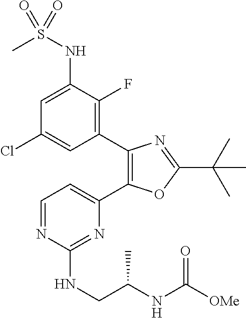 [SMILES: COC(=O)N[C@@H](C)CNc1nccc(-c2oc(C(C)(C)C)nc2-c2cc(Cl)cc(NS(C)(=O)=O)c2F)n1] | (S)-methyl 1-(4-(2-tert-butyl-4-(5- chloro-2-fluoro-3- (methylsulfonamido)phenyl)oxazol- 5-yl)pyrimidin-2-ylamino)propan-2- ylcarbamate |
| P3 | 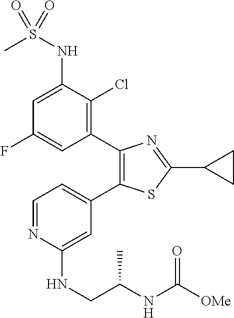 [SMILES: COC(=O)N[C@@H](C)CNc1cc(-c2sc(C3CC3)nc2-c2cc(F)cc(NS(C)(=O)=O)c2Cl)ccn1] | (S)-methyl 1-(4-(4-(2-chloro-5- fluoro-3- (methylsulfonamido)phenyl)-2- cyclopropylthiazol-5-yl)pyridin-2- ylamino)propan-2-ylcarbamate |
| P4 | 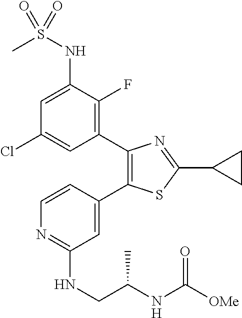 [SMILES: COC(=O)N[C@@H](C)CNc1cc(-c2sc(C3CC3)nc2-c2cc(Cl)cc(NS(C)(=O)=O)c2F)ccn1] | (S)-methyl 1-(4-(4-(5-chloro-2- fluoro-3- (methylsulfonamido)phenyl)-2- cyclopropylthiazol-5-yl)pyridin-2- ylamino)propan-2-ylcarbamate |
| P5 | 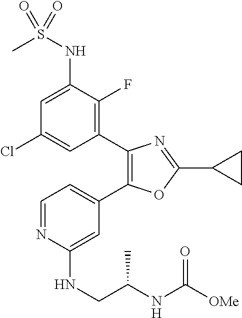 [SMILES: COC(=O)N[C@@H](C)CNc1cc(-c2oc(C3CC3)nc2-c2cc(Cl)cc(NS(C)(=O)=O)c2F)ccn1] | (S)-methyl 1-(4-(4-(5-chloro-2- fluoro-3- (methylsulfonamido)phenyl)-2- cyclopropyloxazol-5-yl)pyridin-2- ylamino)propan-2-ylcarbamate |
| P6 | 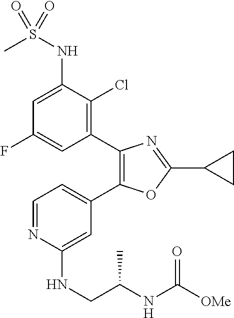 [SMILES: COC(=O)N[C@@H](C)CNc1cc(-c2oc(C3CC3)nc2-c2cc(F)cc(NS(C)(=O)=O)c2Cl)ccn1] | (S)-methyl 1-(4-(4-(2-chloro-5- fluoro-3- (methylsulfonamido)phenyl)-2- cyclopropyloxazol-5-yl)pyridin-2- ylamino)propan-2-ylcarbamate |
| P7 | 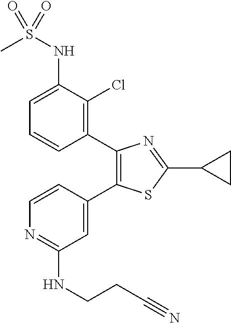 [SMILES: CS(=O)(=O)Nc1cccc(-c2nc(C3CC3)sc2-c2ccnc(NCCC#N)c2)c1Cl] | N-(2-chloro-3-(5-(2-(2- cyanoethylamino)pyridin-4-yl)-2- cyclopropylthiazol-4- yl)phenyl)methanesulfonamide |
| P8 | 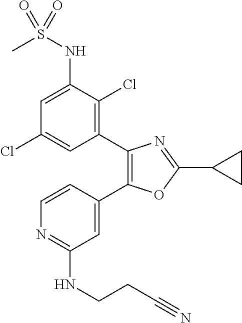 [SMILES: CS(=O)(=O)Nc1cc(Cl)cc(-c2nc(C3CC3)oc2-c2ccnc(NCCC#N)c2)c1Cl] | N-(2,5-dichloro-3-(5-(2-(2- cyanoethylamino)pyridin-4-yl)-2- cyclopropyloxazol-4- yl)phenyl)methanesulfonamide |
| P9 | 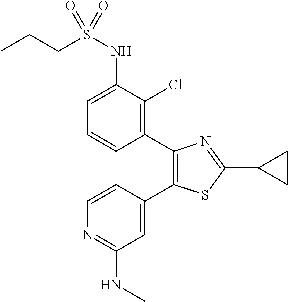 [SMILES: CCCS(=O)(=O)Nc1cccc(-c2nc(C3CC3)sc2-c2ccnc(NC)c2)c1Cl] | N-(2-chloro-3-(2-cyclopropyl-5-(2- (methylamino)pyridin-4-yl)thiazol- 4-yl)phenyl)propane-1-sulfonamide |
| P10 | 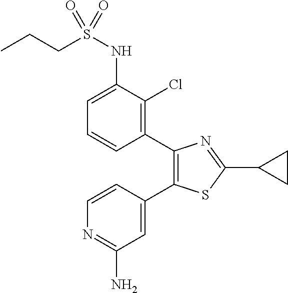 [SMILES: CCCS(=O)(=O)Nc1cccc(-c2nc(C3CC3)sc2-c2ccnc(N)c2)c1Cl] | N-(3-(5-(2-aminopyridin-4-yl)-2- cyclopropylthiazol-4-yl)-2- chlorophenyl)propane-1- sulfonamide |

### PHARMACOLOGICAL DATA

Utility for the compounds of the present invention is supported by the data observed in one or more of the following assays.

Raf/Mek Amplified Luminescence Proximity Homogeneous Assay

(Alpha Screen)

Buffers

Assay buffer: 50 mM Tris, pH 7.5, 15 mM MgCl₂, 0.01% Bovine Serum Albumin (BSA), 1 mM dithiothreitol (DTT)

Stop buffer: 60 mM ethylenediaminetetraacetic acid (EDTA), 0.01% Tween® 20

Bead buffer: 50 mM Tris, pH 7.5, 0.01% Tween® 20

Materials

b-Raf(V600E), active

biotinylated Mek, kinase dead

Alpha Screen detection kit (available from PerkinElmer™, #6760617R)

Anti phospho-MEK1/2 (available from Cell Signaling Technology, Inc. #9121)

384 well assay plates (White Greiner® plates, #781207)

Assay Conditions

b-Raf(V600E) approximately 4 pM

c-Raf approximately 4 nM

biotinylated Mek, Kinase dead approximately 10 nM

ATP 10 μM

Pre-incubation time with compounds 60 minutes at room temperature

Reaction time 1 or 3 hours at room temperature

Assay Protocol

Raf and biotinylated Mek, kinase dead, were combined at 2× final concentrations in assay buffer (50 mM Tris, pH 7.5, 15 mM MgCl₂, 0.01% BSA and 1 mM DTT) and dispensed 10 μl per well in assay plates (Greiner white 384 well assay plates #781207) containing 0.5 μl of 40× of a raf kinase inhibitor test compound diluted in 100% DMSO. The plate was incubated for 60 minutes at room temperature.

The Raf kinase activity reaction was started by the addition of 10 μL per well of 2×ATP diluted in assay buffer. After 3 hours (bRaf(V600E)) or 1 hour (c-Raf), the reactions were stopped with the addition of 10 μL of stop reagent (60 mM EDTA). Phosphorylated product was measured using a rabbit anti-p-MEK (Cell Signaling, #9121) antibody and the Alpha Screen IgG (ProteinA) detection Kit (PerkinElmer #6760617R), by the addition of 30 μL to the well of a mixture of the antibody (1:2000 dilution) and detection beads (1:2000 dilution of both beads) in bead buffer (50 mM Tris, pH 7.5, 0.01% Tween20). The additions were carried out under dark conditions to protect the detection beads from light. A lid was placed on top of the plate and incubated for 1 hour at room temperature, then the luminescence was read on a PerkinElmer Envision instrument. The concentration of each compound for 50% inhibition (IC₅₀) was calculated by non-linear regression using XL Fit data analysis software.

Mutant b-Raf(V600E) IC₅₀ data for representative compounds of the invention in the Raf/Mek Amplified Luminescence Proximity Homogeneous Assay are shown in the Table III below:

TABLE III

| | | mut-bRaf IC₅₀ |
|---|---|---|
| Example | Structure | (uM) |
| 1 | 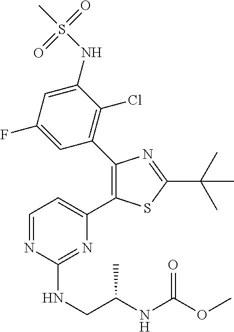 [SMILES: COC(=O)N[C@@H](C)CNc1nccc(-c2sc(C(C)(C)C)nc2-c2cc(F)cc(NS(C)(=O)=O)c2Cl)n1] | 0.00025 |
| 2 | 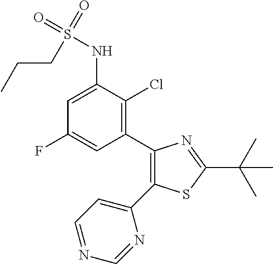 [SMILES: CCCS(=O)(=O)Nc1cc(F)cc(-c2nc(C(C)(C)C)sc2-c2ccncn2)c1Cl] | 0.00044 |
| 3 | 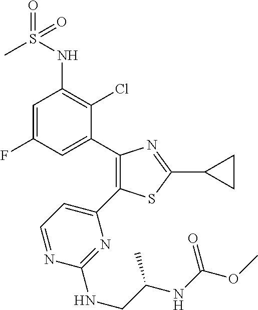 [SMILES: COC(=O)N[C@@H](C)CNc1nccc(-c2sc(C3CC3)nc2-c2cc(F)cc(NS(C)(=O)=O)c2Cl)n1] | 0.00022 |
| 4 | 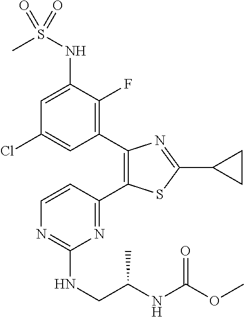 [SMILES: COC(=O)N[C@@H](C)CNc1nccc(-c2sc(C3CC3)nc2-c2cc(Cl)cc(NS(C)(=O)=O)c2F)n1] | 0.00012 |
| 5 | 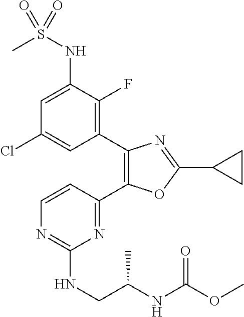 [SMILES: COC(=O)N[C@@H](C)CNc1nccc(-c2oc(C3CC3)nc2-c2cc(Cl)cc(NS(C)(=O)=O)c2F)n1] | 0.00082 |
| 6 | 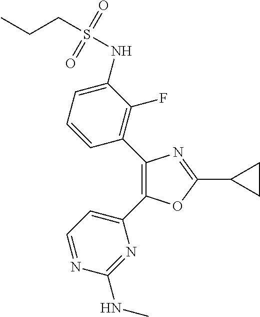 [SMILES: CCCS(=O)(=O)Nc1cccc(-c2nc(C3CC3)oc2-c2ccnc(NC)n2)c1F] | 0.028 |
| 7 | 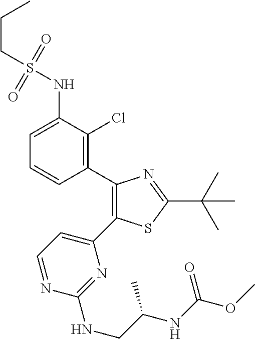 [SMILES: CCCS(=O)(=O)Nc1cccc(-c2nc(C(C)(C)C)sc2-c2ccnc(NC[C@H](C)NC(=O)OC)n2)c1Cl] | 0.00022 |
| 8 | 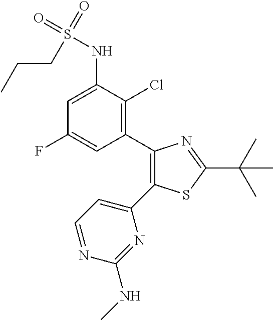 [SMILES: CCCS(=O)(=O)Nc1cc(F)cc(-c2nc(C(C)(C)C)sc2-c2ccnc(NC)n2)c1Cl] | 0.00014 |
| 9 | 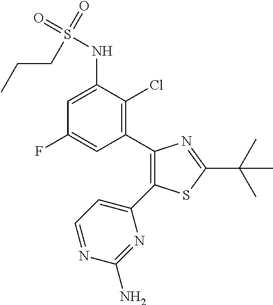 [SMILES: CCCS(=O)(=O)Nc1cc(F)cc(-c2nc(C(C)(C)C)sc2-c2ccnc(N)n2)c1Cl] | 0.00014 |
| 10 | 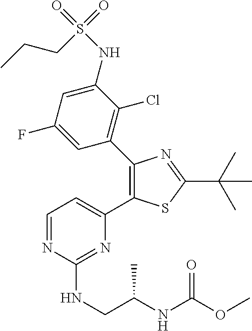 [SMILES: CCCS(=O)(=O)Nc1cc(F)cc(-c2nc(C(C)(C)C)sc2-c2ccnc(NC[C@H](C)NC(=O)OC)n2)c1Cl] | 0.00033 |
| 11 | 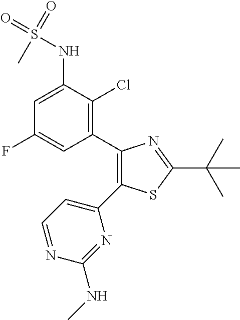 [SMILES: CNc1nccc(-c2sc(C(C)(C)C)nc2-c2cc(F)cc(NS(C)(=O)=O)c2Cl)n1] | 0.0060 |
| 12 | 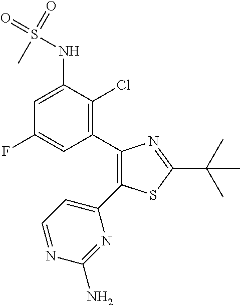 [SMILES: CC(C)(C)c1nc(-c2cc(F)cc(NS(C)(=O)=O)c2Cl)c(-c2ccnc(N)n2)s1] | 0.009 |
| 13 | 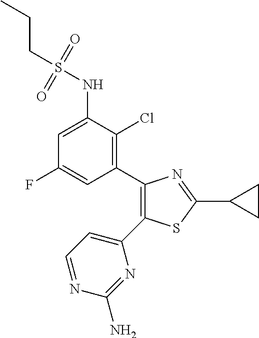 [SMILES: CCCS(=O)(=O)Nc1cc(F)cc(-c2nc(C3CC3)sc2-c2ccnc(N)n2)c1Cl] | 0.00013 |
| 14 | 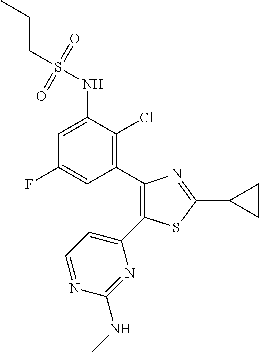 [SMILES: CCCS(=O)(=O)Nc1cc(F)cc(-c2nc(C3CC3)sc2-c2ccnc(NC)n2)c1Cl] | 0.000091 |
| 15 | 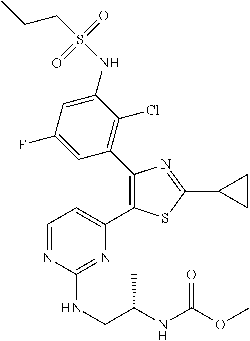 [SMILES: CCCS(=O)(=O)Nc1cc(F)cc(-c2nc(C3CC3)sc2-c2ccnc(NC[C@H](C)NC(=O)OC)n2)c1Cl] | 0.000077 |
| 16 | 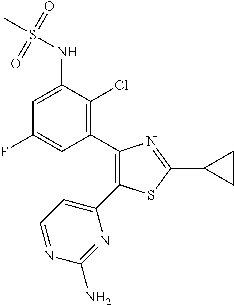 [SMILES: CS(=O)(=O)Nc1cc(F)cc(-c2nc(C3CC3)sc2-c2ccnc(N)n2)c1Cl] | 0.015 |
| 17 | 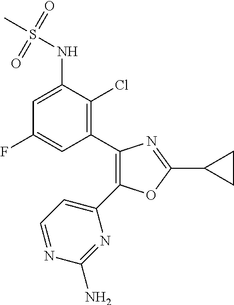 [SMILES: CS(=O)(=O)Nc1cc(F)cc(-c2nc(C3CC3)oc2-c2ccnc(N)n2)c1Cl] | 0.0027 |
| 18 | 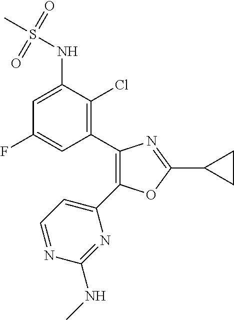 [SMILES: CNc1nccc(-c2oc(C3CC3)nc2-c2cc(F)cc(NS(C)(=O)=O)c2Cl)n1] | 0.018 |
| 19 | 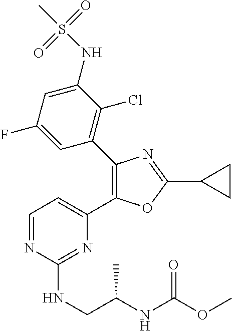 [SMILES: COC(=O)N[C@@H](C)CNc1nccc(-c2oc(C3CC3)nc2-c2cc(F)cc(NS(C)(=O)=O)c2Cl)n1] | 0.0015 |
| 20 | 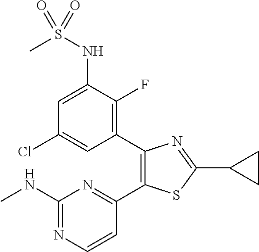 [SMILES: CNc1nccc(-c2sc(C3CC3)nc2-c2cc(Cl)cc(NS(C)(=O)=O)c2F)n1] | 0.0030 |
| 21 | 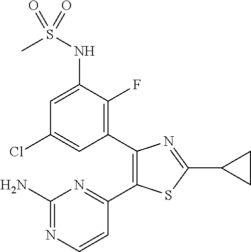 [SMILES: CS(=O)(=O)Nc1cc(Cl)cc(-c2nc(C3CC3)sc2-c2ccnc(N)n2)c1F] | 0.0058 |
| 22 | 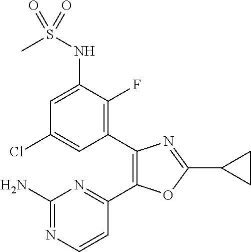 [SMILES: CS(=O)(=O)Nc1cc(Cl)cc(-c2nc(C3CC3)oc2-c2ccnc(N)n2)c1F] | 0.0033 |
| 23 | 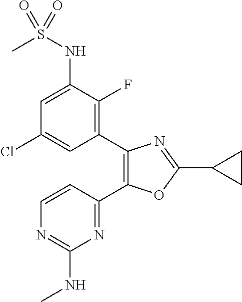 [SMILES: CNc1nccc(-c2oc(C3CC3)nc2-c2cc(Cl)cc(NS(C)(=O)=O)c2F)n1] | 0.018 |
| 24 | 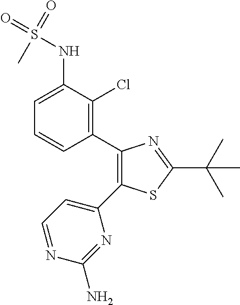 [SMILES: CC(C)(C)c1nc(-c2cccc(NS(C)(=O)=O)c2Cl)c(-c2ccnc(N)n2)s1] | 0.046 |
| 25 | 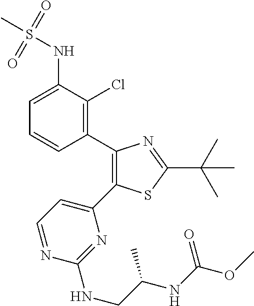 | 0.00032 |
| 26 | 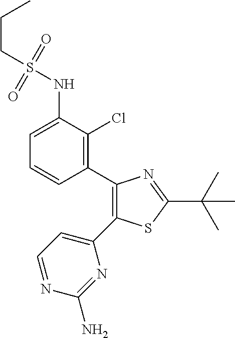 [SMILES: CCCS(=O)(=O)Nc1cccc(-c2nc(C(C)(C)C)sc2-c2ccnc(N)n2)c1Cl] | 0.00065 |
| 27 | 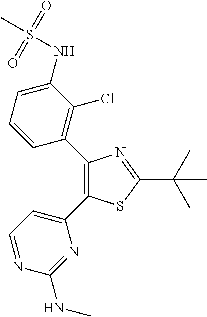 [SMILES: CNc1nccc(-c2sc(C(C)(C)C)nc2-c2cccc(NS(C)(=O)=O)c2Cl)n1] | 0.044 |
| 28 | 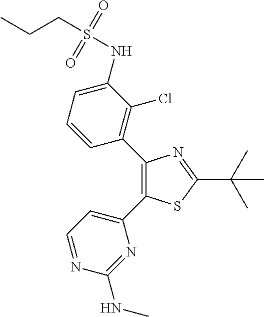 [SMILES: CCCS(=O)(=O)Nc1cccc(-c2nc(C(C)(C)C)sc2-c2ccnc(NC)n2)c1Cl] | 0.0011 |
| 29 | 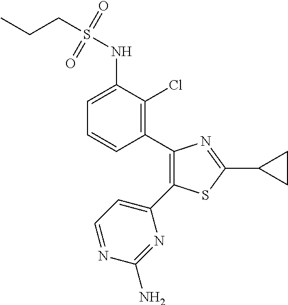 [SMILES: CCCS(=O)(=O)Nc1cccc(-c2nc(C3CC3)sc2-c2ccnc(N)n2)c1Cl] | 0.0034 |
| 30 | 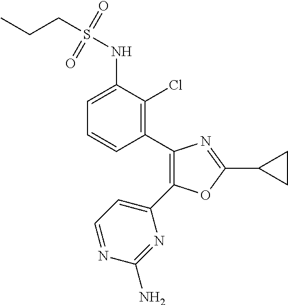 [SMILES: CCCS(=O)(=O)Nc1cccc(-c2nc(C3CC3)oc2-c2ccnc(N)n2)c1Cl] | 0.0012 |
| 31 | 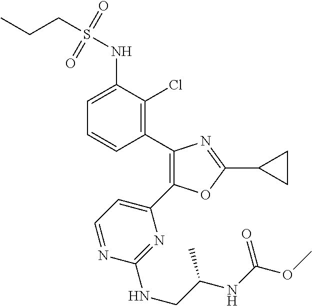 [SMILES: CCCS(=O)(=O)Nc1cccc(-c2nc(C3CC3)oc2-c2ccnc(NC[C@H](C)NC(=O)OC)n2)c1Cl] | 0.0013 |
| 32 | 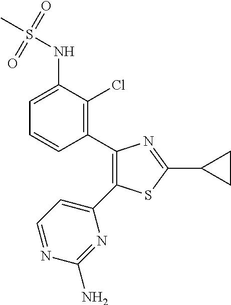 [SMILES: CS(=O)(=O)Nc1cccc(-c2nc(C3CC3)sc2-c2ccnc(N)n2)c1Cl] | 0.044 |
| 33 | 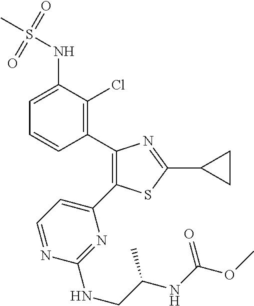 | 0.00042 |
| 34 | 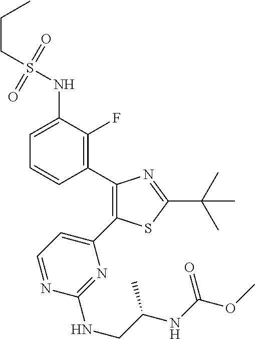 [SMILES: CCCS(=O)(=O)Nc1cccc(-c2nc(C(C)(C)C)sc2-c2ccnc(NC[C@H](C)NC(=O)OC)n2)c1F] | 0.00062 |
| 35 | 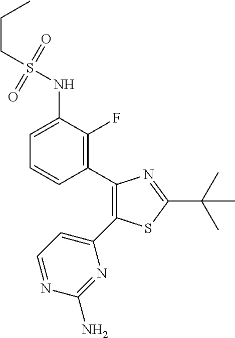 [SMILES: CCCS(=O)(=O)Nc1cccc(-c2nc(C(C)(C)C)sc2-c2ccnc(N)n2)c1F] | 0.0018 |
| 36 | 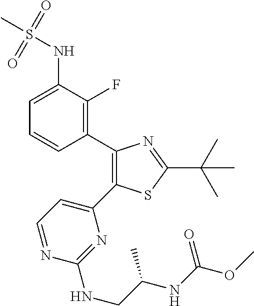 | 0.00043 |
| 37 | 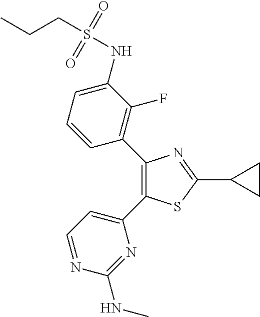 [SMILES: CCCS(=O)(=O)Nc1cccc(-c2nc(C3CC3)sc2-c2ccnc(NC)n2)c1F] | 0.0050 |
| 38 | 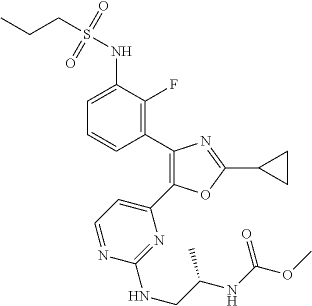 [SMILES: CCCS(=O)(=O)Nc1cccc(-c2nc(C3CC3)oc2-c2ccnc(NC[C@H](C)NC(=O)OC)n2)c1F] | 0.00059 |
| 39 | 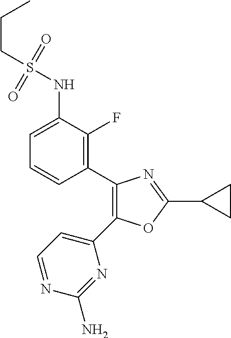 [SMILES: CCCS(=O)(=O)Nc1cccc(-c2nc(C3CC3)oc2-c2ccnc(N)n2)c1F] | 0.0021 |
| 40 | 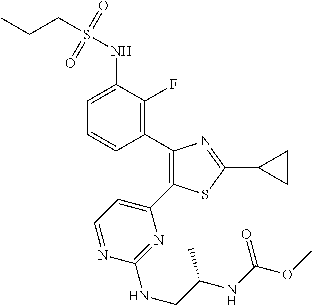 [SMILES: CCCS(=O)(=O)Nc1cccc(-c2nc(C3CC3)sc2-c2ccnc(NC[C@H](C)NC(=O)OC)n2)c1F] | 0.00012 |
| 41 | 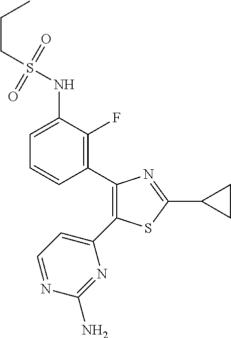 [SMILES: CCCS(=O)(=O)Nc1cccc(-c2nc(C3CC3)sc2-c2ccnc(N)n2)c1F] | 0.011 |
| 42 | 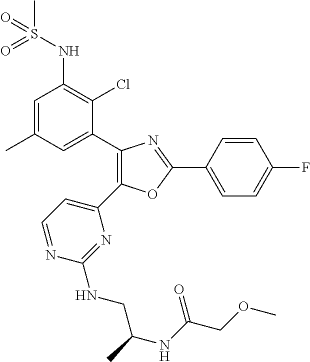 [SMILES: COCC(=O)N[C@@H](C)CNc1nccc(-c2oc(-c3ccc(F)cc3)nc2-c2cc(C)cc(NS(C)(=O)=O)c2Cl)n1] | 0.0031 |
| 43 | 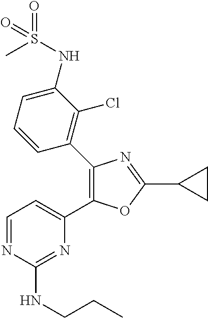 [SMILES: CCCNc1nccc(-c2oc(C3CC3)nc2-c2cccc(NS(C)(=O)=O)c2Cl)n1] | 0.062 |
| 44 | 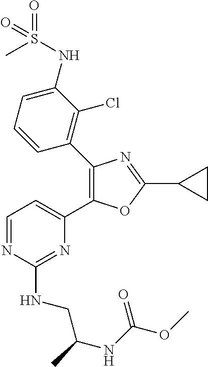 [SMILES: COC(=O)N[C@@H](C)CNc1nccc(-c2oc(C3CC3)nc2-c2cccc(NS(C)(=O)=O)c2Cl)n1] | 0.0013 |
| 45 | 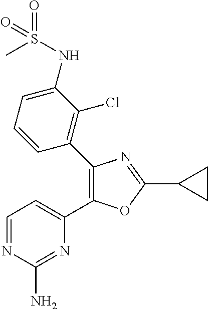 [SMILES: CS(=O)(=O)Nc1cccc(-c2nc(C3CC3)oc2-c2ccnc(N)n2)c1Cl] | 0.017 |
| 46 | 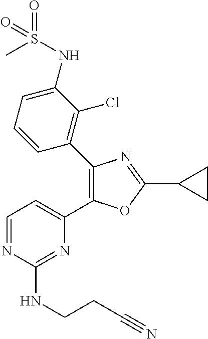 [SMILES: CS(=O)(=O)Nc1cccc(-c2nc(C3CC3)oc2-c2ccnc(NCCC#N)n2)c1Cl] | 0.00095 |
| 47 | 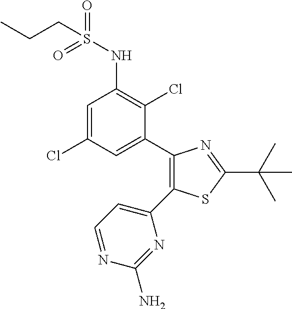 [SMILES: CCCS(=O)(=O)Nc1cc(Cl)cc(-c2nc(C(C)(C)C)sc2-c2ccnc(N)n2)c1Cl] | 0.00013 |
| 48 | 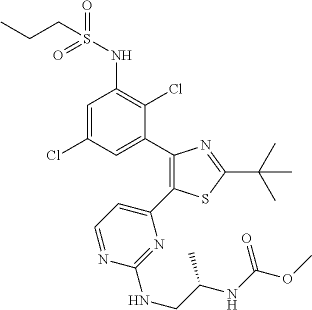 [SMILES: CCCS(=O)(=O)Nc1cc(Cl)cc(-c2nc(C(C)(C)C)sc2-c2ccnc(NC[C@H](C)NC(=O)OC)n2)c1Cl] | 0.00070 |
| 49 | 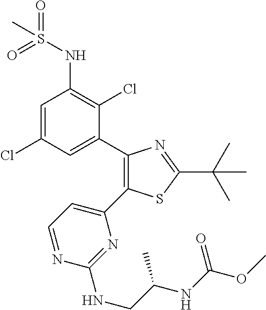 [SMILES: COC(=O)N[C@@H](C)CNc1nccc(-c2sc(C(C)(C)C)nc2-c2cc(Cl)cc(NS(C)(=O)=O)c2Cl)n1] | 0.00040 |
| 50 | 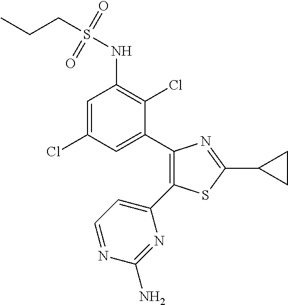 [SMILES: CCCS(=O)(=O)Nc1cc(Cl)cc(-c2nc(C3CC3)sc2-c2ccnc(N)n2)c1Cl] | 0.000056 |
| 51 | 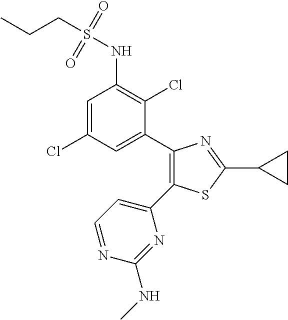 [SMILES: CCCS(=O)(=O)Nc1cc(Cl)cc(-c2nc(C3CC3)sc2-c2ccnc(NC)n2)c1Cl] | 0.000080 |
| 52 | 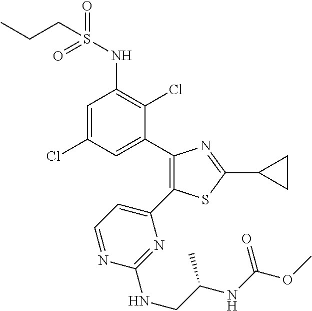 [SMILES: CCCS(=O)(=O)Nc1cc(Cl)cc(-c2nc(C3CC3)sc2-c2ccnc(NC[C@H](C)NC(=O)OC)n2)c1Cl] | 0.00024 |
| 53 | 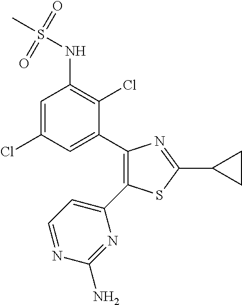 [SMILES: CS(=O)(=O)Nc1cc(Cl)cc(-c2nc(C3CC3)sc2-c2ccnc(N)n2)c1Cl] | 0.00045 |
| 54 | 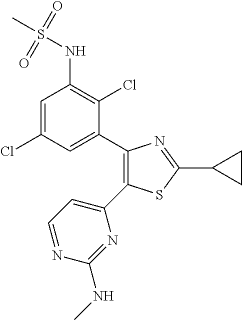 [SMILES: CNc1nccc(-c2sc(C3CC3)nc2-c2cc(Cl)cc(NS(C)(=O)=O)c2Cl)n1] | 0.00046 |
| 55 | 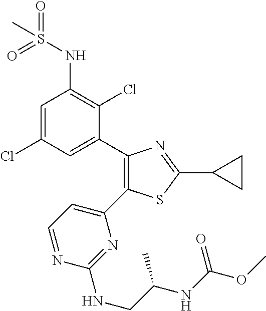 [SMILES: COC(=O)N[C@@H](C)CNc1nccc(-c2sc(C3CC3)nc2-c2cc(Cl)cc(NS(C)(=O)=O)c2Cl)n1] | 0.00028 |
| 56 | 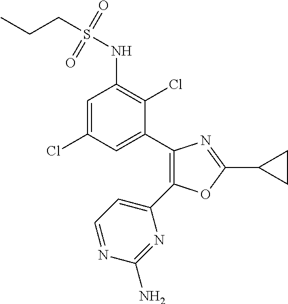 [SMILES: CCCS(=O)(=O)Nc1cc(Cl)cc(-c2nc(C3CC3)oc2-c2ccnc(N)n2)c1Cl] | 0.000022 |
| 57 | 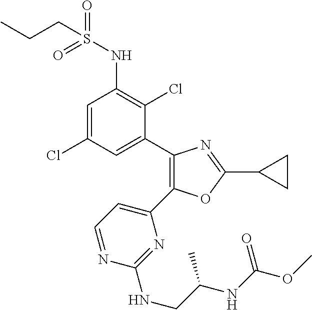 [SMILES: CCCS(=O)(=O)Nc1cc(Cl)cc(-c2nc(C3CC3)oc2-c2ccnc(NC[C@H](C)NC(=O)OC)n2)c1Cl] | 0.00056 |
| 58 | 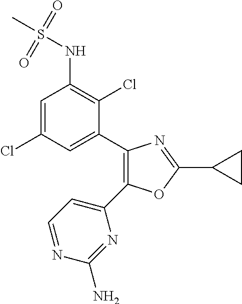 [SMILES: CS(=O)(=O)Nc1cc(Cl)cc(-c2nc(C3CC3)oc2-c2ccnc(N)n2)c1Cl] | 0.00035 |
| 59 | 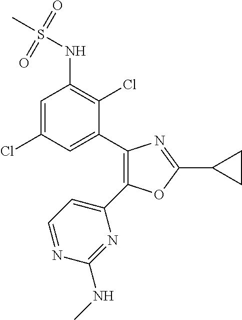 [SMILES: CNc1nccc(-c2oc(C3CC3)nc2-c2cc(Cl)cc(NS(C)(=O)=O)c2Cl)n1] | 0.0015 |
| 60 | 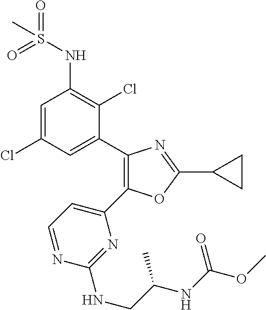 | 0.00046 |
| 61 | 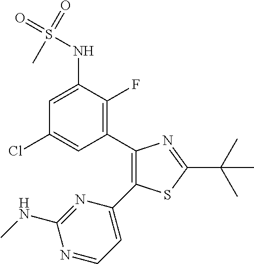 [SMILES: CNc1nccc(-c2sc(C(C)(C)C)nc2-c2cc(Cl)cc(NS(C)(=O)=O)c2F)n1] | 0.00065 |
| 62 | 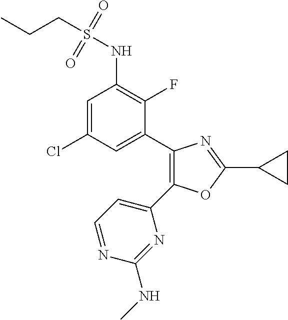 [SMILES: CCCS(=O)(=O)Nc1cc(Cl)cc(-c2nc(C3CC3)oc2-c2ccnc(NC)n2)c1F] | 0.00028 |
| 63 | 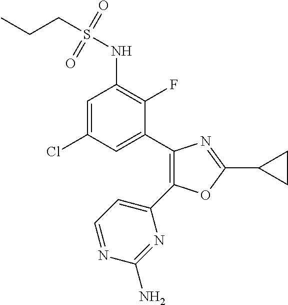 [SMILES: CCCS(=O)(=O)Nc1cc(Cl)cc(-c2nc(C3CC3)oc2-c2ccnc(N)n2)c1F] | 0.000044 |
| 64 | 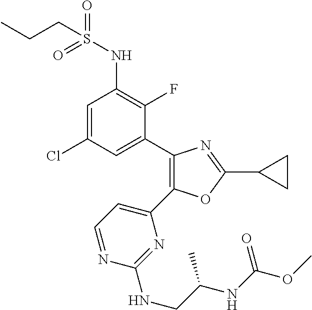 [SMILES: CCCS(=O)(=O)Nc1cc(Cl)cc(-c2nc(C3CC3)oc2-c2ccnc(NC[C@H](C)NC(=O)OC)n2)c1F] | 0.00028 |
| 65 | 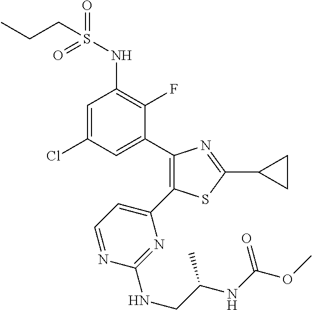 [SMILES: CCCS(=O)(=O)Nc1cc(Cl)cc(-c2nc(C3CC3)sc2-c2ccnc(NC[C@H](C)NC(=O)OC)n2)c1F] | 0.000090 |
| 66 | 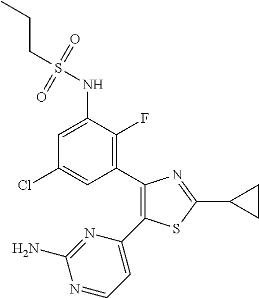 [SMILES: CCCS(=O)(=O)Nc1cc(Cl)cc(-c2nc(C3CC3)sc2-c2ccnc(N)n2)c1F] | 0.000058 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula I: 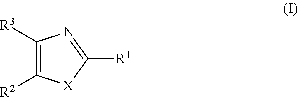
or a pharmaceutically acceptable salt thereof, wherein:
X represents O or S;
R¹ is selected from C₁₋₆-alkyl, C₃₋₈ branched alkyl, C₃₋₈ cycloalkyl, optionally substituted heteroaryl, optionally substituted heterocyclyl, and optionally substituted aryl;
R² is heteroaryl substituted with R¹¹;
R³ is phenyl substituted with R¹², R¹³, and R¹⁵;
R¹¹ is selected from H, and optionally substituted amino;
R¹² is halogen and H;
R¹³ is NHSO₂alkyl; and
R¹⁵ is selected from halogen, H, and C₁₋₆ alkyl.

2. A compound of Formula I: 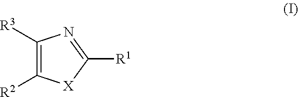
or a pharmaceutically acceptable salt thereof, wherein:
X represents O or S;
R¹ is selected from C₃₋₆ branched alkyl, C₃₋₆ cycloalkyl, and optionally substituted phenyl;
R² is heteroaryl substituted with R¹¹;
R³ is phenyl substituted with R¹², R¹³, and R¹⁵;
R¹¹ is selected from H, amino, and NH—CH₂—CH(CH₃)NH—C(O)—OCH₃;
R¹² is halogen;
R¹³ is NHSO₂C₁₋₆ alkyl; and
R¹⁵ is selected from halogen, H, and C₁₋₆ alkyl.

3. A compound of Formula I: 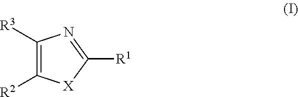
or a pharmaceutically acceptable salt thereof, wherein:
X represents O or S;
R¹ is selected from C₃₋₆ branched alkyl, C₃₋₆ cycloalkyl, and optionally substituted phenyl;
R² is heteroaryl substituted with R¹¹;
R³ is phenyl substituted with R¹², R¹³, and R¹⁵;
R¹¹ is selected from H, NH(CH₂)₁₋₂—CN, and amino;
R¹² is halogen;
R¹³ is NHSO₂ C₁₋₆ alkyl; and
R¹⁵ is selected from halogen, H, and C₁₋₆ alkyl.

4. (canceled)

5. A compound of claim 3, wherein:
X represents O or S;
R¹ is selected from t-butyl, cyclo-propyl, and substituted phenyl;
R² is pyrimidinyl substituted with R¹¹;
R³ is phenyl substituted with R¹², R¹³, and R¹⁵;
R¹¹ is NH₂;
R¹² is Cl or F;
R¹³ is NHSO₂—C₁₋₃ alkyl; and
R¹⁵ is selected from F, Br, CH₃, H, and Cl.

6. A compound of claim 5 wherein X represents O.

7. A compound of claim 5 wherein X represents S.

8. A compound of claim 6 wherein:
R¹ represents cyclopropyl; and
R¹⁵ represents Cl or F.

9. (canceled)

10. The compound of claim 1 selected from the group consisting of:
N-(3-(5-(2-aminopyrimidin-4-yl)-2-cyclopropylthiazol-4-yl)-5-chloro-2-fluorophenyl)propane-1-sulfonamide; (S)-methyl 1-(4-(4-(5-chloro-2-fluoro-3-(propylsulfonamido)phenyl)-2-cyclopropylthiazol-5-yl)pyrimidin-2-ylamino)propan-2-ylcarbamate; N-(2,5-dichloro-3-(2-cyclopropyl-5-(2-(methylamino)pyrimidin-4-yl)thiazol-4-yl)phenyl)propane-1-sulfonamide;
N-(3-(5-(2-aminopyrimidin-4-yl)-2-cyclopropylthiazol-4-yl)-2,5-dichlorophenyl)propane-1-sulfonamide;
(S)-methyl 1-(4-(4-(2-chloro-5-fluoro-3-(propylsulfonamido)phenyl)-2-cyclopropylthiazol-5-yl)pyrimidin-2-ylamino)propan-2-ylcarbamate; and
N-(2-chloro-3-(2-cyclopropyl-5-(2-(methylamino)pyrimidin-4-yl)thiazol-4-yl)-5-fluorophenyl)propane-1-sulfonamide; N-(3-(5-(2-aminopyrimidin-4-yl)-2-cyclopropyloxazol-4-yl)-5-chloro-2-fluorophenyl)propane-1-sulfonamide; and
N-(3-(5-(2-aminopyrimidin-4-yl)-2-cyclopropyloxazol-4-yl)-2,5-dichlorophenyl)propane-1-sulfonamide.
or a pharmaceutically acceptable salt thereof.

11. A pharmaceutical composition comprising a compound of claim 1, and a diluent, carrier or excipient.

12. The pharmaceutical composition of claim 11 further comprising an additional therapeutic agent, wherein said additional therapeutic agent is selected from the group consisting of an anticancer compound, an analgesic, an antiemetic, an antidepressant, and an anti-inflammatory agent.

13.-14. (canceled)

15. A method to treat cancer, comprising administering to a subject in need of such treatment a pharmaceutically effective amount of a compound of claim 1.

16. The method of claim 15, wherein said cancer is selected from the group consisting of lung carcinoma, pancreatic carcinoma, bladder carcinoma, colon carcinoma, myeloid disorders, melanomas, and adenomas.

17. The method of claim 15, further comprising administering to the subject an additional therapeutic agent.

18. The method of claim 17, wherein the additional therapeutic agent comprises an anticancer drug, a pain medication, an antiemetic, an antidepressant or an anti-inflammatory agent.

19. The method of claim 18, wherein the additional therapeutic agent is a different Raf kinase inhibitor or an inhibitor of MEK, mTOR, PI3K, CDK9, PAK, Protein Kinase C, a MAP kinase, a MAPK Kinase, or ERK.

20. The method of claim 19, wherein the additional therapeutic agent is administered to the subject concurrently with the compound.

21. A method to treat a condition mediated by b-Raf(V600E), comprising administering to a subject in need thereof an effective amount of a compound according to claim 1.

22.-36. (canceled)
